(19) United States
(12) Patent Application Publication
(10) Pub. No.: US 2024/0400998 A1
(43) Pub. Date: Dec. 5, 2024

(54) BLASTOCYST-LIKE CELL AGGREGATE AND METHODS

(71) Applicant: IMBA - INSTITUT FÜR MOLEKULARE BIOTECHNOLOGIE GMBH, Wien (AT)

(72) Inventors: Nicolas RIVRON, Wien (AT); Harunobu KAGAWA, Wien (AT); Alok JAVALI, Wien (AT); Heidar HEIDARI KHOEI, Wien (AT)

(21) Appl. No.: 18/260,418

(22) PCT Filed: Jan. 13, 2022
(86) PCT No.: PCT/EP2022/050593

§ 371 Date: Jul. 5, 2023

(30) Foreign Application Priority Data
Jan. 13, 2021 (EP) 21151455.9
Oct. 1, 2021 (EP) 21200473.3

(51) Int. Cl. C12N 5/071

(52) U.S. Cl. CPC C12N 5/0682; C12N 2501/999; C12N 2502/02; C12N 2503/02; C12N 2506/02; C12N 2513/00

(57) ABSTRACT

A method of generating a blastoid or a blastocyst-like structures by aggregation and culture of cells includes culturing an aggregate of human pluripotent stem cells (hPSCs) and trophoblast cells in a medium comprising a HIPPO pathway inhibitor in a 3D culture; blastoids and a blastocyst-like cell aggregates obtainable by the methods and uses thereof; as well as similar treatments of blastocyst for in vitro fertilization that increase blastocyst development and implantation; as well as similar contraceptive treatment of human embryos that decrease blastocyst development and implantation.

The present invention relates to the generation of blastocyst-like structures by aggregation and culture of cells.

### BACKGROUND OF THE INVENTION

Understanding of human early embryonic development and implantation are extremely limited due to the small number of studied human embryos and to the difficulties to experimentally manipulate them physically and genetically. Other experimental model organisms have been used for studying these early development and implantation mechanisms, typically mice. However, it has now become clear that there are important differences between humans and other species in terms of morphology and molecules at play, which renders the study of human embryogenesis necessary.

EP 2 986 711 A1 relates to the generation of a blastoid using at least one trophoblast cell and at least one pluripotent cell.

WO 2018/175691 A1 concerns the generation of totipotent cells.

WO 2020/262531 A1 describes producing primordial endoderm stem cells by culturing a blastocyst.

RONGHUI Li et al., Cell, Elsevier, Vol. 179 (3), 2019:687 describes generation of a blastocyst-like structure from a single stem-cell type.

RIVRON Nicolas C et al., Nature, MacMillan Journ. Ltd., Vol. 557 (7703), 2018:106-111 describes the generation of embryonic day 3.5 blastocysts from trophoblast and embryonic stem cells.

VRIJ Erik J. et al., bioRxiv, DOI: 10.1101/510396 describes a combinatorial screen of proteins, GPCR ligands and small molecules to rapidly guide embryoid bodies to form a three-dimensional primitive endoderm-/epiblast-like niche.

KIME Cody et al., Stem Cell Reports, 13 (3), 2019:485-498 describes induced self-organizing 3D blastocyst-like cysts (iBLCs) generated from mouse pluripotent stem cell culture.

Altogether, there is still a compelling need for in vitro alternatives to the use of human embryos for research. Models are needed that can be made widely available, are amenable to easy genetic manipulations and high-throughput drug screens.

Accordingly, it is a goal of the invention to provide such models.

### SUMMARY OF THE INVENTION

The present invention provides a method of generating a blastoid or a blastocyst-like cell aggregate or blastocyst-like structure comprising culturing an aggregate of human pluripotent stem cells (hPSCs) and trophoblast cells in a medium comprising a HIPPO pathway inhibitor in a 3D culture. These blastoids can be used for high-throughput genetic or drug screens in the context of drug development. This method can also be used for triggering a pregnancy. The components of the medium and molecules revealed by the use of blastoids can also be used to modulate the behaviour of blastocysts, for example during in vitro fertilization procedure, in order to improves blastocyst development and implantation.

The invention further provides a kit suitable for culturing a blastoid, comprising a HIPPO pathway inhibitor, a MEK inhibitor, and a TGF-beta inhibitor. One or more of these compounds can be combined in a medium for culturing human pluripotent stem cells (hPSCs).

The invention further provides blastoids and a blastocyst-like cell aggregates obtainable by said methods.

Also provided is a blastoid or blastocyst-like cell aggregate comprising an outer epithelial monolayer of trophoblast-like cells, preferably characterized by the expression of for example GATA3 and CDX2, surrounding at least one fluid-filled cavity and at least one inner cluster of cells comprising epiblast-like, preferably characterized by the expression of for example Nanog and Oct4, and hypoblast-like cells, preferably characterized by the expression of for example GATA4, wherein the outer epithelial monolayer comprises polar-like trophoblasts that express NR2F2.

The invention further provides an in vitro method of increasing or testing the potential of a blastoid or a blastocyst to implant into a layer of endometrial cells, comprising stimulating the endometrial cells with a Wnt inhibitor, preferably XAV939 and/or LF3, contacting the blastoid or blastocyst with the layer of stimulated endometrial cells; in the method of testing further measuring the level of attachment, invasion, and differentiation to the trophoblasts, blastoid or blastocyst into the endometrium cells.

The invention further provides a Wnt inhibitor for use in a method of increasing the chance of a blastocyst implantation, for example during an in vitro fertilization procedure, comprising contacting the blastocyst with an endometrium in the presence of the Wnt inhibitor or stimulating the endometrium with a Wnt inhibitor before transferring the blastocyst in utero or to the endometrium.

Related thereto, the invention provides a method of increasing the chance of a blastocyst to implant, for example during an in vitro fertilization procedure, comprising contacting the blastocyst with an endometrium in the presence of the Wnt inhibitor or stimulating the endometrium with a Wnt inhibitor before transferring the blastocyst in utero or to the endometrium. Also provided is the use of a Wnt inhibitor for manufacturing a pharmaceutical composition for mediating blastocyst implantation, e.g. during an in vitro fertilization procedure, that comprises contacting the blastocyst with an endometrium in the presence of the Wnt inhibitor or stimulating the endometrium with a Wnt inhibitor before transferring the blastocyst in utero or to the endometrium.

Further provided is a HIPPO pathway inhibitor for use in a method of producing a blastocyst suitable for implantation, for example to improve the quality of a blastocyst during an in vitro fertilization, comprising treating an embryo in an early stage, selected from the morula stage or blastocyst stage until a mature blastocyst stage, with the HIPPO pathway inhibitor and letting the embryo in a morula stage grow into the blastocyst stage or letting the embryo in the blastocyst stage grow into a more mature blastocyst stage.

Related thereto, the invention provides a method of producing a blastocyst suitable for in vitro fertilization, comprising treating an embryo in an early stage, selected from 1-cell stage, 2-cells stage, 4-cells stage, 8-cells stage or 16-cells stage, the morula stage or blastocyst stage until a mature blastocyst stage, with the HIPPO pathway inhibitor and letting the embryo in a morula stage grow into the blastocyst stage or letting the embryo in the blastocyst stage grow into a more mature blastocyst stage. The 1-cell stage, 2-cells stage, 4-cells stage, 8-cells stage or 16-cells stage can also be referred to as the cleavage stages. Also provided is the use of a HIPPO pathway inhibitor for the manufacture of a pharmaceutical composition for producing a blastocyst suitable for implantation, for example to improve the developmental potential of a blastocyst during an in vitro fertilization procedure, which comprises treating an embryo in an early stage, selected from the 1-cell stage, 2-cells stage, 4-cells stage, 8-cells stage or 16-cells stage, morula stage or blastocyst stage until a mature blastocyst stage, with the HIPPO pathway inhibitor and letting the embryo in a 1-cell stage, 2-cells stage, 4-cells stage, 8-cells stage or 16-cells stage, or morula stage grow into the blastocyst stage or letting the embryo in the blastocyst stage grow into a more mature blastocyst stage.

All aspects of the invention are related and any disclosure of specific embodiments for one aspect also relate to other aspects. E.g. a disclosure of treatments of a blastoid or a blastocyst-like cell aggregate in vitro can also be done for the treatments in vivo for preparation of blastocyst for preparation in an in vitro fertilization procedure and the preceding treatments steps. Any compound described for the inventive methods may be part of the kit. The components of the kit and the kit may be used in the inventive methods and treatments.

### DETAILED DESCRIPTION OF THE INVENTION

The invention includes a method to form blastocyst-like cell aggregates, generally termed blastoids, from human pluripotent stem cells (hPSCs). Contrary to in vivo blastocysts, blastoids can be produced in large numbers and are amenable to genetic and drug screens, while alleviating some ethical concerns related to the manipulation of human embryos given that the artificial blastocyst-like cell aggregates and blastoids are not able to form or develop into human embryos.

Blastocyst-like cell aggregates and blastoids are human embryo models that have an important potential for biomedical discoveries, including for drug safety/efficacy and for therapies of early pregnancy (e.g., improving in vitro fertilization procedures and contraceptives).

The inventive blastoids can form the first axis and their epiblast induces the maturation of the polar trophectoderm that consequently acquires the potential to specifically attach to hormonally-stimulated endometrial cells. Such human blastoids are faithful, scalable, versatile, and ethical models to explore human implantation and development.

As used herein, the terms blastocyst-like cell aggregates, blastocyst-like structure and blastoids are used interchangeably to reflect the tissues obtainable by the inventive methods that model blastocyst without being true blastocyst. The term blastocyst is reserved to such embryos.

Blastoids recapitulate the three-dimensional morphological and molecular signatures of the human blastocyst including the concomitant specification and spatial organization of tissues reflecting the three founding lineages that form the whole organism, namely the trophectoderm, the epiblast and the hypoblast.

A high-fidelity and high-efficiency model of the human blastocyst would support scientific and medical progress. However, its predictive capacity depends on its ability to faithfully recapitulate the sequences of blastocyst cellular specification and morphogenesis according to the natural developmental pace. Accurate modelling ensures the formation of cells reflecting the blastocyst stage only, as well as the in vitro recapitulation of aspects of implantation and peri-implantation development.

The invention also describes treating (human) blastocysts to prepare them for improved implantation chances, e.g. during in vitro fertilization (IVF) procedures or treating a patient after a natural conception to improve the chances of pregnancy. Such treatments may be medical or therapeutic in nature to treat the human embryo or the recipient mother. For such methods, the invention also relates to manufacturing pharmaceutical compositions with the compounds for the treatment (e.g. a HIPPO pathway inhibitor) or said compounds for use in the treatment. Human embryos as such or their uses for industrial or commercial purposes may not be part of the invention.

As a core aspect, the present invention provides a method of generating a blastoid or a blastocyst-like cell aggregate comprising culturing an aggregate of human pluripotent stem cells (hPSCs) and trophoblast cells in a medium comprising a HIPPO pathway inhibitor in a 3D culture. Preferably, the human pluripotent stem cells (hPSCs) are surrounded or get surrounded by the trophoblast cells.

According to the invention, the formation of blastoids according to the invention achieves forming three-dimensional aggregates of hPSCs and modulating the activity of the HIPPO pathway, which triggers the concomitant specification and three-dimensional self-organization of epiblast-, trophectoderm- and hypoblast-like cells, along with the formation of an embryonic-abembryonic axis. Accordingly, a HIPPO pathway inhibitor is used as a core aspect of the invention to generate the blastoids.

The invention provides for the first time the induction of the concomitant formation of the three cell types of (i) epiblast-like, (ii) trophectoderm-like, and (iii) hypoblast-like cells from hPSCs and their self-organization into structures morphologically and molecularly similar to the human blastocyst, thereby recapitulating the 3D morphological change constrained by the concomitant cell lineage segregation, morphogenesis, and maturation of tissues reflecting the trophectoderm, epiblast and hypoblast.

The resulting blastoids can actively interact with a layer of endometrial cells in vitro or the endometrium, the lining cells of the uterus in vivo, when it is made receptive, for example upon stimulation with Estrogen, Progesterone, CAMP, and the Wnt-inhibitor XAV939 and/or LF3. Similar to blastocysts, the attachment, invasion, and differentiation of blastoids to the endometrial cells occurs predominantly via the polar trophectoderm, meaning the trophoblasts that juxtapose the epiblast cells. Upon implantation, the polar trophoblasts of the blastoids proliferate, differentiate, and produce human chorionic gonadotropin, the hormone used to signify a clinical pregnancy.

Accordingly, the inventive method is useful for
1. genetic and drug screens for understanding and managing early pregnancy;
2. development of contraceptive and fertility drugs;
3. development of IVF culture conditions;
4. in vitro toxicity/safety assays for drug development;
5. in vitro formation of specific cells, tissues, and organs for in vitro assays and in vivo transplantations.

The present invention comprises the step of culturing an aggregate of human pluripotent stem cells (hPSCs) and of trophoblast cells in a medium comprising a HIPPO pathway inhibitor in a 3D culture. Preferably, the human pluripotent stem cells (hPSCs) get surrounded by the trophoblast cells in this method step or human pluripotent stem cells (hPSCs) are surrounded by the trophoblast cells through a preceding step, e.g. an optional step of culturing aggregated hPSCs in a medium comprising a HIPPO pathway inhibitor, in which trophoblast cells may form surrounding the hPSCs.

The term “HIPPO pathway inhibitor” and “HIPPO pathway antagonist” are used interchangeably herein. This term refers to a compound that reduces activity of the HIPPO pathway. The HIPPO pathway is reviewed in Gumbiner and Kim, Journal of Cell Science (2014) 127, 709-717 (incorporated herein by reference). The HIPPO pathway exercises inhibitory action on the ability of Hippo-Yes-associated protein (YAP) to shuttle to the nucleus. One such inhibitory action is through phosphorylation of YAP, which prevents YAP from entering the nucleus. Compounds that prevent or reduce YAP phosphorylation in a cell are thus suitable HIPPO pathway inhibitors. By inhibiting the HIPPO pathway, the inhibition on YAP is removed or reduced, leading to an increased activity of YAP in the nucleus and by consequence usually to cell proliferation (see e.g. FIG. 4 of Gumbiner and Kim). As such the HIPPO pathway inhibitor of the invention may also be a YAP activator, i.e. leading to increased YAP activity in the nucleus. As such, a HIPPO pathway inhibition includes YAP activation and HIPPO pathway inhibitor include YAP activators. An example YAP activation is e.g. overexpression of YAP in a cell, such as by using a recombinant nucleic acid expressing YAP as YAP activator. Such a nucleic acid, can be administered to a cell, e.g. using a vector, for YAP activation. A Hippo pathway inhibitor is XMU-MP-1 (Triastuti et al., Br J Pharmacol. 2019; 176:3956-3971), which is preferably used in lower concentrations according to the inventive methods of preparing a blastoid and preparing a blastoid or blastocyst for implantation into an endometrium. XMU-MP-1 is an exceptionally potent Hippo pathway inhibitor. High amounts of XMU-MP-1, e.g. more than 1 μM or about 2 μM and more in culture, may be used to induce the formation of trophectoderm-like cells and to form a blastoid with a limited number of or no inner cells (so-called trophosphere). XMU-MP-1 is a strong inhibitor of the Hippo pathway with stronger and more long-lasting effects than LPA. As a result, the aggregate largely forms trophoblasts at the expense of forming epiblasts and hypoblast cells. Blastoids with inner cells form at lower amounts of XMU-MP-1, e.g. about 1 μM or less in culture. With optimization of concentration of a Hippo pathway inhibitor, the length of exposure of the cells to the Hippo inhibitor, the combination of the Hippo inhibitor with additional molecules, and the initial number of cells the result can be controlled in general. XMU-MP-1 is an inhibitor of MST1/2. The invention provides the use of XMU-MP-1 or an inhibitor of MST1/2 in a method of generating a trophosphere (which may lack inner cells) that does not have the potential to attach and invade endometrial cells—this method may be in vitro; and/or in a method of contraception. Sufficient amount of XMU-MP-1 and time of exposure of the cells to XMU-MP-1 may be used to obtain a trophosphere, e.g. 2 μM or more and an exposure of 4 days or more. Also provided is a MST1/2 inhibitor, preferably XMU-MP-1 for use as a contraceptive.

A preferred HIPPO pathway inhibitor is a ligand of the lysophosphatidic acid receptor (LPAR), in particular preferred lysophosphatidic acid itself (LPA, e.g. 1-Oleoyl lysophosphatidic acid). A further preferred HIPPO pathway inhibitor and a ligand of the lysophosphatidic acid receptor is NAEPA or OEA-P (oleoyl ethanolamide phosphate), N-[2-(phosphonooxy)ethyl]-9Z-octadecenamide. These are a lysophosphatidic acid (LPA) mimetics. The ligand of the LPAR may be an activator or agonist of the LPAR. As further or alternative LPAR ligands any derivative of LPA may be used. Derivatives of LPA are preferably compounds of formula 1:

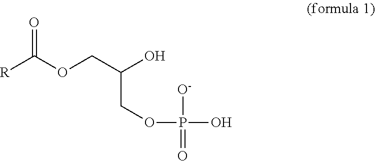

wherein R is a C₈-C₂₄-alkyl, a C₈-C₂₄-alkenyl or C₈-C₂₄-alkynyl. Preferably R is a C₉-, C₁₀-, C₁₁-, C₁₂-, C₁₃-, C₁₄-, C₁₅-, C₁₆-, C₁₇-, C₁₈-, C₁₉-, C₂₀-, C₂₁-, C₂₂-, C₂₃-alkenyl, -alkyl or -alkynyl. A preferred compound is (2-hydroxy-3-phosphonooxypropyl) (Z)-octadec-9-enoate.

The LPAR is preferably LPAR1, LPAR2, LPAR3, LPAR4, LPAR5 or LPAR6. In particular preferred the LPAR is LPAR2.

Further LPAR ligands are GRI977143 and any derivatives thereof as disclosed in WO 2014/036038 A1 (incorporated herein by reference). Such ligands include compounds of formula 2:

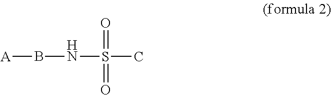

wherein A is

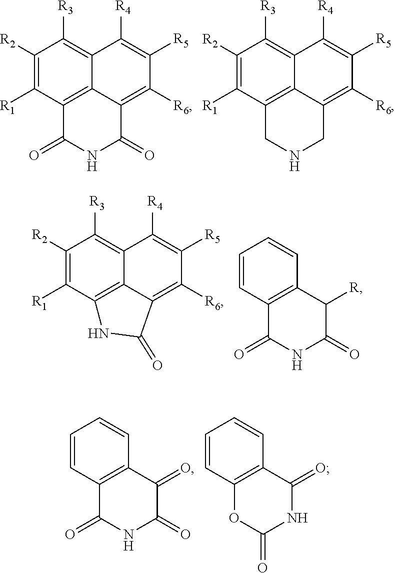

R is H or substituted or unsubstituted phenyl;
R₁, R₂, R₃, R₄, R₅, and Re are independently H, NO₂, Br, Cl, or OCH₃;
B is C₂-C₈-alkyl or -alkenyl; and C is

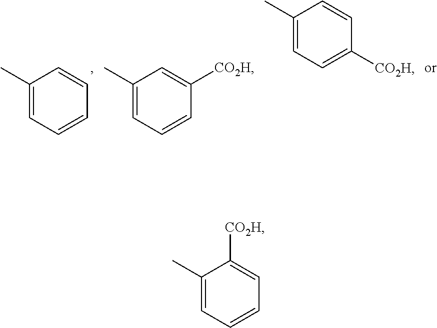

optionally substituted with F, Cl, Br, NO₂, NH₂, OCH₃, CH₃, CO₂H, or phenyl. For example, the compound can be 2-((9-oxo-9H-fluoren-2-yl) carbamoyl)benzoic acid, 2-((3-(1,3-dioxo-1H-benzo[de]isoquinolin-2 (3H)-yl) propyl)thio)benzoic acid, 4,5-dichloro-2-((9-oxo-9H-fluoren-2-yl) carbamoyl)benzoic acid or 2-((9,10-dioxo-9,10-dihydroanthracen-2-yl) carbamoyl)benzoic acid.

Alternative or combinable HIPPO pathway inhibitors may be a Mst1 inhibitor, a Mst2 inhibitor or a combined Mst1 and Mst2 inhibitor, such as XMU-MP-1 (Triastuti et al., supra) or a Lats kinase inhibitor, such as TRULI (Kastan et al., bioRxiv, 2020, doi.org/10.1101/2020.02.11.944157). The Lats kinase inhibitor may be an ATP-competitive inhibitor of Lats kinases. Lats kinase is involved in YAP phosphorylation (Gumbiner et al, supra) and inhibition of Lats activity therefore decrease YAP inactivation by phosphorylation and increases YAP activity in the nucleus.

The HIPPO pathway inhibitor may be a YAP activator, in particular a YAP activator that reduces or prevents YAP phosphorylation and/or facilitates entry of YAP into the nucleus of a cell. A further preferred HIPPO pathway inhibitor for use according to the invention is verteporfin.

A 3D culture is a culture that allows tissue development in all three dimensions. Contrary thereto, in a 2D culture cells are induced to grow attached onto a surface and are deterred from growing away from said surface, with such growth not necessarily being excluded. 2D culturing may induce cell layer formation, such as mono-layers, bilayers or multilayers and/or two-dimensional cell expansion; 3D culturing usually allows growth in all directions equally, wherein of course the cell tissues are allowed to develop a tissue directionality or axis by themselves. Layer formation in 2D cultures may be induced by gravity and/or adhesion between cells or to a surface. Conditions for 2D and 3D cultures may be influenced by the type of surface, e.g. adherent surface for 2D cultures and non-adherent surface for 3D cultures, the medium, the lack (2D) or presence (3D) or a scaffolding 3D matrix, such as a gel structure, e.g. hydrogel. 2D culture may comprise feeder cells as adherent surface.

A medium for growing the cells and tissue allows unhindered development to a blastoid. A medium may comprise nutrients, such as one or more carbohydrates, amino acids and salts. An example medium is B27N2 medium (Sunwoldt et al., Front. Mol. Neurosci. 10, 2017:305). The medium preferably comprises insulin. Alternatively or in combination, the medium may comprise holotransferin, selenite, corticosterone or progesterone, retinol or a combination thereof. Such a medium may also be provided with the inventive kit.

The inventive method comprises providing an aggregate of human pluripotent stem cells (hPSCs) and trophoblast cells. hPSCs are pluripotent cells that are able to differentiate into the trophectoderm-like tissue, epiblast-like tissue and hypoblast-like tissue. The suffix “-like” indicates that although the tissues will resemble trophectoderm, epiblast and hypoblast, respectively, these tissues will usually not develop identically as in an in vivo situation since the inventive blastoids are still artificial constructs. However, the “-like” tissues of the blastoid of the invention will usually express similar expression markers as the in vivo counterparts and can be identified similarly.

The aggregate of human pluripotent stem cells (hPSCs) and trophoblast cells can be provided by common techniques, such as disclosed in WO 2014/171824 A1 or in Okae et al., Cell Stem Cell 22, 2018:50-63 (both incorporated herein by reference). In a preferred method according to the invention, the aggregate of hPSCs and trophoblasts can be generated by culturing aggregated hPSCs in a medium comprising any one selected from a HIPPO pathway inhibitor, a MEK inhibitor and a TGF-beta inhibitor. Preferably a MEK inhibitor and a TGF-beta inhibitor are used. Preferably also a HIPPO pathway inhibitor, is used.

In particular preferred embodiments, a “triple inhibition” of the HIPPO pathways (as above), of MEK/ERK and of TGF-beta is used in the generation of the inventive blastoid. Especially, the inventive method comprises culturing an aggregate of human pluripotent stem cells (hPSCs) and trophoblast cells in a medium comprising a HIPPO pathway inhibitor in a 3D culture. In the triple inhibition, it is preferred that the aggregate of hPSCs and trophoblasts is generated by culturing aggregated hPSCs in a medium comprising a MEK inhibitor and a TGF-beta inhibitor, especially further preferred also comprising a HIPPO pathway inhibitor at this stage.

Naive human pluripotent stem cells (hPSCs) that are inhibited for the Hippo, TGF-β, and MEK/ERK pathways efficiently (>70%) form blastoids with the 3 founding lineages (>97% trophectoderm, epiblast, and primitive endoderm) according to the sequence and pace of blastocyst development.

The triple inhibition leads to a breakage of the symmetry in the aggregated cells, that leads to the formation of a unique inner cluster of epiblast and primitive endoderm cells that remains attached to one side of a trophoblast cyst. As a direct consequence, the asymmetry created within the cyst by the presence of this unique inner cluster induces the local maturation of polar trophoblasts and defines the direction of the attachment to endometrial cells. The inventive blastoids may thus have one localized inner cluster of epiblast and primitive endoderm cells attached to one side of a trophoblast cyst.

MEK (also called ERK) inhibition, by e.g. a MEK inhibitor, can be used for inducing the formation of trophoblasts from hPSCs as disclosed in Guo et al., biorxiv 2020, doi.org/10.1101/2020.02.04.933812. If cell aggregates with trophoblast cells are already provided, a MEK inhibition is not needed. If no trophoblast cells are present, these can be formed by using the MEK inhibitor. The MEK inhibitor can be a MAPK inhibitor, e.g. SB202190.

The MEK inhibitor is preferably PD0325901. PD0325901 can be a compound of formula 3:

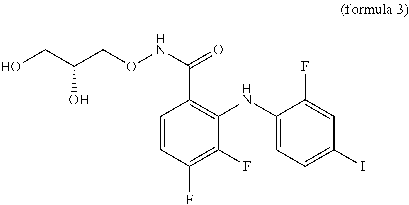 [SMILES: O=C(NOC[C@H](O)CO)c1ccc(F)c(F)c1Nc1ccc(I)cc1F]

Further or alternative MEK inhibitors may be selected from Selumetinib, Mirdametinib, Trametinib, U0126-EtOH, PD184352 (CI1040), PD98059, Pimasertib, TAK-733, AZD8330 (ARRY704), Binimetinib, PD318088, SL327, Refametinib, GDC-0623 (G-868), Cobimetinib.

The TGF-beta inhibitor may be an inhibitor of TGF-β type I receptor. The TGF-beta inhibitor is preferably A83-1 (A83-01, A83-01). A83-1 can be a compound of formula 4:

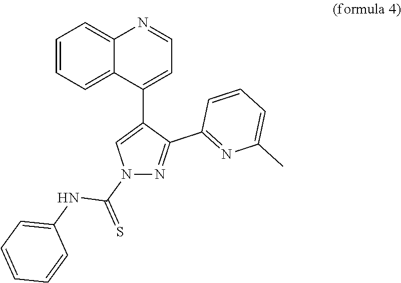 [SMILES: Cc1cccc(-c2nn(C(=S)Nc3ccccc3)cc2-c2ccnc3ccccc23)n1]

Further or alternative TGF-beta inhibitors may be selected from SD-208, GW788388, SRI-011381, TP0427736, RepSox (E-616452, SJN 2511), LY2109761, SB505124, BIBF-0775, LY 3200882, Galunisertib (LY2157299), Vactosertib (TEW-7197, EW-7197), LY364947 (HTS 466284), SB525334, ITD-1 or SB431542. SB431542 is particularly preferred.

The aggregated hPSCs (for the above step of generating an aggregate of hPSCs and trophoblast cells) can be formed by seeding hPSCs and aggregating the seeded hPSCs by culturing in a growth medium. The hPSCs to be seeded are preferably dissociated hPSCs. hPSCs may e.g. be dissociated by trypsinization. Dissociated hPSCs are not aggregated into one combined tissue. However, they are able to aggregate later during development in the inventive method.

These method steps to generate different stages and intermediaries during the formation of the inventive blastoid can be combined, e.g. when it is desired to produce the aggregate of hPSCs and trophoblast cells in situ. As such, the blastoid can be formed from hPSCs in one culture. The same basic media may be used for growth and nutrient supply (e.g. B27N2 or others) but different additional compounds are used during different steps. The inventive method may comprise the combination of
(i) seeding hPSCs and aggregating the seeded hPSCs by culturing in a growth medium to form the aggregated hPSCs,
(ii) culturing aggregated hPSCs in a medium comprising a MEK inhibitor and a TGF-beta inhibitor to generate the hPSCs and trophoblast cells; optionally a HIPPO pathway inhibitor is also used in step (ii);
(iii) culturing the aggregate of hPSCs and trophoblast cells in a medium comprising a HIPPO pathway inhibitor in a 3D culture to generate the blastoid or a blastocyst-like cell aggregate.

The invention includes combinations of steps (i), (ii) and (iii), but also a combination of steps (ii) and (iii), the latter e.g. if aggregated hPSCs can be provided (without the surrounding trophoblast cells that are the starting point for step (iii)).

In step (iii), the MEK inhibitor and/or the TGF-beta inhibitor may not be used. In step (i) or in step (ii) the HIPPO pathway inhibitor may not be used. Also in step (i), alternatively or in combination, the MEK inhibitor and/or the TGF-beta inhibitor may not be used. In step (ii), the use of the TGF-beta inhibitor and MEK/ERK inhibitor is sufficient to form blastoids in step (iii).

The aggregated hPSCs (for the step of generating an aggregate of hPSCs and trophoblast cells) are preferably formed by seeding hPSCs and aggregating the seeded hPSCs by culturing in a growth medium with culturing in a growth medium for 0 to 64 hours or for 0 to 12 hours or for 12 to 64 hours as a preferment of step (i). Aggregating the seeded hPSCs is an optional step as the method also works without this step. In a preferred embodiment of step (i), combinable or as alternative, the growth medium comprises a ROCK inhibitor. A preferred ROCK inhibitor is Y27632 (Y-27632). Further or alternative ROCK inhibitors may be selected from ZINC00881524, Thiazovivin, Fasudil (HA-1077), GSK429286A (RHO-15), RKI-1447, Azaindole 1 (TC-S 7001), GSK269962A HCl (GSK269962B, GSK269962), Hydroxyfasudil (HA1100), Netarsudil (AR-13324), Ripasudil (K-115), Y-39983 (Y33075), KD025 (SLx-2119). The ROCK inhibitor increases or improves aggregation of the seeded hPSCs.

In a preferment, culturing of seeded hPSCs in the growth medium (e.g. step (i) mentioned above) comprises seeding 1 to 200 hPSCs, preferably 20 to 150 hPSCs, in particular preferred 30 to 120 hPSCs, even more preferred 30 to 60 hPSCs, in a vessel and growing said seeded hPSCs in the growth medium. This number of cells leads to an optimal blastoid formation in later steps.

In preferred embodiments, the treatment and or growth of the seeded hPSCs is or has been done in a 2D culture environment. 2D cultures may lead to a two-dimensional cell expansion as mentioned above.

Preferably, the hPSCs during the 2D culture, (before the 3D culture), have been treated with a MEK inhibitor and/or a PKC inhibitor, e.g. as described in Guo et al., Development 2017, doi: 10.1242/dev.146811. A Wnt inhibitor and a STAT activator may also be used in combination or alternatively as mentioned above.

In preferred embodiments, the PKC inhibitor is selected from Gö6983 (GOE 6983) and Ro-31-8425 or a combination thereof. Further or alternative PKC inhibitors may be selected from Enzastaurin (LY317615), Sotrastaurin (AEB071), Mitoxantrone (NSC-301739), Staurosporine (CGP 41251), Bisindolylmaleimide I (GF109203X, GO 6850), Bisindolylmaleimide IX (Ro 31-8220), LXS196 (IDE-196), VTX-27, Midostaurin (pkc412, CGP 41251), Chelerythrine, Go6976 (PD406976), CRT0103390.

In preferred embodiments, the 2D cultured hPSCs further is or have been treated with a Wnt inhibitor and/or a STAT agonist. Such a treatment results in more naïve hPSCs or hPSCs in a ground state. Such naïve or ground state hSPCs are preferably used as hPSCs in the inventive method in step (i). Such a treatment to produce more naïve or ground state hPSCs is e.g. disclosed in Takashima et al., Cell 158 (6), 2014:1254-1269 or WO 2016/027099 A2 (both incorporated herein by reference). The STAT agonist is preferably a STAT3 agonist, e.g. LIF.

An example Wnt inhibitor is XAV939. XAV939 may be a compound of formula (5):

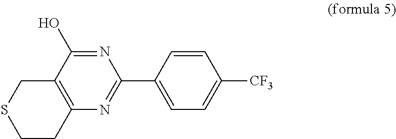 [SMILES: Oc1nc(-c2ccc(C(F)(F)F)cc2)nc2c1CSCC2]

Further or alternative Wnt inhibitors may be selected from LF3, PKF118-310, Wnt3A, Adavivint (SM04690), CCT251545, PNU-75654, IWP-2, IWP-3, IWR-1-endo, iCRT3, WIKI4, ICG-001, XAV-939 (NVP-XAV939), LGK-974 (NVP-LGK974, WNT974), MSAB, KYA1797K, JW55, or combinations thereof. LF3 and/or XAV939 are particular preferred. Particular preferred Wnt inhibitors for all embodiments of the invention are XAV939, IWP2, PNU74654 and LF3.

In a particular preferred embodiment, the hPSCs may be pre-treated in a medium containing inhibitors of MEK, Wnt, PKC, and an against or activator of STAT (e.g. LIF) as e.g. described in Guo et al., Development 2017, doi: 10.1242/dev.146811 and Takashima et al., supra. A medium termed PXGL maintains hPSCs in a more naïve state. This more naïve state improves the formation of blastoids. Many culture conditions to make hPSCs naïve are known in the art and can be used according to the invention, e.g. as described in WO 2016/027099 A2.

In preferred embodiments, the aggregated cells (e.g. step (ii) mentioned above) are cultured for at least 1 day, preferably at least 2 days.

The above embodiments are preferably combined, e.g. in an example, a culture starting from hPSCs comprises culturing the cells at least 5 days: the first 0 to 24 hours or the first day is in medium without small molecule inhibitors (without a MEK inhibitor (e.g. PD0325901), a TGF-beta inhibitor (e.g. A83-01), an activator of STAT (e.g. LIF), and a HIPPO pathway inhibitor (e.g. LPA)) as discussed above (except for a ROCK inhibitor, e.g. Y27632, which favors the aggregation of the cells). This is an example of step (i) and anything said above for step (i) also applies here. Between 0 and 24 hours, or on the second day, the treatment with a MEK inhibitor (e.g. PD0325901), a TGF-beta inhibitor (e.g. A83-01), an activator of STAT (e.g. LIF), a ROCK pathway inhibitor, and a HIPPO pathway inhibitor (e.g. LPA) is started. This is an example of step (ii) and anything said above for step (ii) also applies here. The same medium is used on the third day (step (ii)). On the fourth day, the cells/aggregates are cultured in a medium containing only the ROCK pathway inhibitor and the HIPPO pathway inhibitor (e.g. LPA) from the discussed small molecule inhibitors, e.g. the MEK inhibitor (e.g. PD0325901), a TGF-beta inhibitor (e.g. A83-01), an activator of STAT (e.g. LIF) are not used anymore. This is an example of step (iii) and anything said above for step (iii) also applies here. The blastoids are usually fully formed on day 5. This method may be in vitro for example to enhance the development of blastocyst issued from In Vitro Fertilization; and/or in a method of enhancing fertility during the first weeks of pregnancy. The Hippo inhibitor, preferably LPA or NAEPA, may be used for enhancing blastocyst development and potential. Also provided is a Hippo inhibitor, preferably LPA or NAEPA, for use as a fertility enhancer. The Hippo inhibitor, preferably LPA or NAEPA, may be administered to a patient 1 to 12 days after conception, preferably 2 to 9 days after conception, in particular preferred 3 to 7 days after conception.

By modifying growth conditions, preferred times for each step may vary. In preferred embodiments, step (i) is for 18 to 48 hours; preferably step (ii) is for 36 to 92 hours; preferably step (iii) is for 18 to 48 hours. These times are also preferred embodiments if step (i) or steps (i) and (ii) are not done, e.g. if aggregated hPSCs or an aggregate of hPSCs and trophoblast cells are used as a starting point.

The hPSCs (human pluripotent stem cells) are preferably not human totipotent cells, i.e. they are not human embryos.

The hPSCs may be from any cell line. Preferably the cell line is selected from hESC H9, Shef6, HNES1, hiPSC CR-NCRM2, and hiPSC niPSC16.2.b.

Preferably, the cells, aggregated hPSCS or the aggregate of hPSCs and trophoblast cells, selected independently, are seeded or placed into a microwell. A microwell may be used to control the number of cells. The microwell may be in an array to allow a plurality of parallel blastoid formations. Preferably, at least 2, e.g. 3, 4, 5, 6, 7, 8, 9 or 10 or more, such as 50 or more blastoids are created in parallel by the inventive method.

Preferably, the 3D culture, e.g. the culturing of the aggregate of hPSCs and trophoblast cells, is done by culturing in a non-adherent vessel, preferably by culturing in microwells, especially preferred by microwells comprising a non-adherent surface made of hydrogel.

In preferred embodiments, the medium for culturing in a 3D culture, in particular the culturing the aggregate of hPSCs and trophoblast cells (such as step (iii) mentioned above), and/or the medium for culturing aggregated hPSCs (such as step (ii) mentioned above) further comprises a STAT3 agonist. The STAT3 agonist is preferably leukemia inhibitory factor (LIF). LIF is preferably human LIF.

In preferred embodiments the hPSCs and trophoblasts (e.g. step (iii) mentioned above) are cultured for at least 16 hours, preferably at least 20 hours, even more preferred at least 1 day, possible are also at least 2 days.

Preferably, the culturing of the cells, in particular the hPSCs and trophoblast cells, is at least until formation of a trophectoderm-like tissue, an epiblast-like tissue and a hypoblast-like tissue out of the aggregate of hPSCs and trophoblasts. Alternatively or in combination, culturing may be at least until formation of an embryonic-abembryonic axis.

Alternatively, the culturing of the cells, in particular the hPSCs and trophoblast cells, is done in the presence of the MST1/2 inhibitor XMU-MP-1 that favours the formation of trophoblasts and disfavours the formation or the maintenance of the epiblast-like cells and hypoblast-like cells, when in sufficient concentration of XMU-MP-1, until the formation of trophoblast cysts that contain a smaller inner cluster or do not contain an inner cluster of epiblast-like and hypoblast-like cells. These trophoblast cysts are termed XMU-MP-1-Trophospheres.

The invention provides the use of XMU-MP-1 or an inhibitor of MST1/2 in a method of generating a trophosphere (which may lack inner cells)—this method may be in vitro; and/or in a method of contraception. The MST1/2 inhibitor, preferably XMU-MP-1, may be used for contraception. Also provided is a MST1/2 inhibitor, preferably XMU-MP-1 for use as a contraceptive. The MST1/2 inhibitor, preferably XMU-MP-1 may be administered to a patient 1 to 9 days after conception, preferably 2 to 7 days after conception, in particular preferred 3 to 6 days after conception.

Alternatively, the culturing the cells, in particular the hPSCs and trophoblast cells, is done in the presence of the STAT inhibitor SC144 that disfavours the formation and maintenance of the epiblast-like cells and hypoblast-like cells until the formation of trophoblast cysts that do not contain an inner cluster of epiblast-like cells and hypoblast-like cells or contain a decreased number of epiblast-like cells and hypoblast-like cells. These trophoblast cysts are termed SC144-Trophospheres. As described above, a STAT agonist may be used in certain steps of the inventive method. In order to prevent or limit the formation of a blastoid with inner cells, a STAT inhibitor, like SC144, can be used. The STAT inhibitor, preferably SC144, may be used for contraception. Also provided is a STAT inhibitor, preferably SC144, for use as a contraceptive. The STAT inhibitor, preferably SC144, may be administered to a patient 1 to 9 days after conception, preferably 2 to 7 days after conception, in particular preferred 3 to 6 days after conception.

Trophoblasts are cells that form the outer layer of a blastocyst or blastoid, and would develop into a large part of the placenta in vivo. The term trophectoderm describes the epithelial cystic tissue that forms the outer layer of the blastocyst. In the blastoid, an outer tissue resembles such trophectoderm and is referred to as trophectoderm-like tissue.

Epiblast is one of two distinct layers arising from the inner cell mass in the mammalian blastocyst. It derives the embryo proper through its differentiation into the three primary germ layers, ectoderm, mesoderm and endoderm, during gastrulation. In the blastoid, an inner tissue develops that resembles such epiblast and is referred to as epiblast-like tissue.

The hypoblast, is one of two distinct layers arising from the inner cell mass in the mammalian blastocyst. The hypoblast gives rise to the yolk sac, which in turn gives rise to the chorion. In the blastoid, an inner tissue develops that resembles such hypoblast and is referred to as hypoblast-like tissue.

Expression patterns of epiblast-like, trophoblast-like and hypoblast-like cells have been clustered as shown in FIG. 9d. Expression markers of epiblast-like cells are for example TDGF1, GDF3, SUSD2, POU5F1, PRDM14, DPPA4 and/or DNMT3L. Expression markers of trophoblast-like cells are for example KRT19, CLDN4, GATA2, KRT18 and/or HAND1. Expression markers of hypoblast-like cells are for example PDGFRA, COL4A1, COL4A2, GATA6 and/or LAMA1. Gene names and gene symbols are for genes as set forth by the HUGO gene nomenclature committee (www.genenames.org). Expression patterns can e.g. be determined by determining mRNA expression.

The formation of the embryonic-abembryonic axis is a preferred embodiment as the blastocyst and blastoids implants via the trophoblasts that juxtapose the inner aggregate of epiblasts/hypoblasts. These so-called polar trophoblasts are characterized by the expression of NR2F2+ and/or CCR7+. Preferably, the inventive blastoid comprises polar trophoblasts expressing NR2F2+ and/or CCR7+.

Preferably the cells, in particular the hPSCs and trophoblast cells, are cultured at least until formation of a three-dimensional cell aggregate with an overall diameter of at least 100 μm, preferably at least 140 μm, even more preferred 180 μm to 220 μm, formed by an outer epithelial monolayer of trophoblast-like cells surrounding a fluid-filled cavity and at least one inner cluster of cells comprising epiblast-like and hypoblast-like cells. The inventive blastoid may comprise any or all of these properties.

In a further embodiment the blastoid generated by the inventive method can be seeded onto a layer of endometrial cells. The seeding is preferably done in vitro. The blastoid may be allowed to implant into or onto a layer of endometrial cells. This implantation is a process the blastoid can do by itself, if not artificially inhibited. The layer of endometrial cells may be a monolayer.

Preferably the endometrial cells or the endometrium in an in vitro fertilization method have/has been treated with a compound selected from estrogen, estrone, estriol, ethinyl estradiol, 17α-ethylnylestradiol, mestranol, progesterone, a progestin, CAMP, and a Wnt-inhibitor, preferably XAV939, IWP2 (also referred to as IWP-2), PNU-74654, and LF3. Such a treatment improves receptiveness for blastoid or blastocyst implantation into or onto the endometrial cells. In particular preferred is the treatment with a Wnt-inhibitor, preferably XAV939, or any of the Wnt inhibitors mentioned above. This treatment with a Wnt inhibitor can be combined with a treatment with estrogen, estrone, estriol, ethinyl estradiol, 17α-ethylnylestradiol, mestranol, progesterone, a progestin and/or CAMP.

In particular preferred is the inhibition of Wnt for preparing a layer of endometrial cells or an endometrium in the uterus in the course of in vitro fertilization for blastoid or blastocyst implantation. It increases receptiveness of the endometrial cells or endometrium for implantation.

The seeding of the blastoid onto a layer of endometrial cells can be used to study effects on implantation quality, effectiveness and/or inhibition. In general, any stage of the inventive method can be used to study the development or abilities or properties of a blastoid as a model of blastocyst development or of a blastocyst, or abilities or properties.

The inventive method can be used for testing or screening a candidate compound and/or candidate genetic alteration and/or even environmental effects, such as temperature, on having an effect at blastoid formation and/or implantation of a blastoid into a layer of endometrial cell. Such a method may comprise treating the aggregate with at least one candidate compound and/or providing the aggregate with at least one candidate genetic alteration and performing the method of the invention. The effects of this method may be compared to the method without the respective at least one candidate compound and/or at least one candidate genetic alteration and/or altered environmental effect as control comparison. Otherwise, the control comparison is performed likewise as is common for controls in order to evaluate the effect of the at least one candidate compound and/or at least one candidate genetic alteration and/or environmental effects only.

For successful implantation and subsequent development to occur, distinct tissues (polar trophoblast and mural trophoblast) form on each side of the trophoblast cyst. These tissues are thought to play different roles (e.g., adhesion, induction of proliferation) during the interaction with the endometrium and uterine tissues. Accordingly, human blastocysts implant via the polar tissue. The inventive method can be used to study the development of these distinct tissues and implantation therewith. Candidate compounds and/or candidate genetic alterations and/or environmental effects can be studied if they influence this tissue formation or implantation thereof.

For successful implantation and subsequent development to occur, the inner cluster of epiblast-like and hypoblast-like cells secretes molecules that are inducing the outer trophoblasts. These molecular inductions are thought to play different roles (e.g. proliferation, differentiation, mechanical action) to endow trophoblasts with the capacity to interact with the endometrium and uterine tissues. Accordingly, human blastocysts implant via the polar tissue. The inventive method can be used to study the role of the molecular inducers in endowing the trophoblasts to interact with the endometrial and uterine tissues. Candidate molecular inducers and/or candidate genetic alterations and/or environmental effects can be studied if they influence these molecular inducers and their effects on trophoblasts or implantation thereof.

The present invention also provides a blastoid obtainable by a method of the invention. The invention provides a blastoid comprising an outer epithelial monolayer of trophoblast-like cells surrounding at least one fluid-filled cavity and at least one inner cluster of cells comprising epiblast-like and hypoblast-like cells, wherein the outer epithelial monolayer comprises polar trophoblasts that express NR2F2. Any of the above-described property, cell type, tissue type may be part of the inventive blastoid, such as a fluid-filled cavity, which may be free of immobilized cells. It may also be free of disseminated cells or may comprise disseminated cells.

The present invention further provides a kit suitable for culturing a blastoid. Any component or combination thereof mentioned above may be in the kit. The kit may preferably comprise a HIPPO pathway inhibitor, a MEK inhibitor and/or a TGF-beta inhibitor. These compounds are preferably combined in a medium for human pluripotent stem cells (hPSCs). The compounds may be for use in any step as mentioned above. The kit preferably further comprises a Wnt inhibitor, e.g. XAV-939. Any of the above-described inhibitors may be used, with the indicated preferred inhibitors also being preferred for the inventive kit. A particularly preferred HIPPO pathway inhibitor is LPA.

The kit may comprise any compound selected from PD0325901 (a MEK inhibitor), Go6983 (a PKC inhibitor), XAV-939 (a Wnt inhibitor), A83-01 (a TGF-beta inhibitor), or combinations thereof, as preferred examples of a MEK inhibitor, a PKC inhibitor, a Wnt inhibitor and a TGF-beta inhibitor, respectively.

The kit may also comprise a ROCK inhibitor.

The kit may also comprise any growth factor selected from LIF, IGF-1, IL-6, IL-11, FGF2, FGF4 or combinations thereof. LIF may be used as described above. IGF-1 and/or IL-6 and/or IL-11 and/or FGF2 and/or FGF4 may be used to improve aggregate cell growth in a medium of the invention during any one of stages (i), (ii), (iii), or combinations thereof.

The kit may also describe a medium as described above. In particular preferred, the kit comprises insulin.

As discussed above, the present invention has shown that the use of a Wnt inhibitor improves implantation of a blastoid onto a layer of endometrial cells. Accordingly, the invention provides an in vitro method of increasing the potential of implanting a blastoid or blastocyst into a layer of endometrial cells, comprising treating the blastoid or blastocyst with a Wnt inhibitor, preferably XAV939, and contacting the blastoid or blastocyst with the layer of endometrial cells. This method can also be used to test at least one candidate compound and/or at least one candidate genetic alteration and/or environmental effects similar as described above to test thereof on implantation or the development of the blastoid after implantation or the endometrial cells after implantation, e.g. in comparison to a control without such as at least one candidate compound and/or at least one candidate genetic alteration and/or environmental effect.

The improvement of using a Wnt inhibitor can also be used in vivo and/or in vitro during an in vitro fertilization (IVF) method.

Provided is a Wnt inhibitor for use in a method of increasing the chance of a blastocyst implantation during in vitro fertilization, comprising contacting the blastocyst with an endometrium in the presence of the Wnt inhibitor, preferably XAV939; preferably wherein the endometrium is contacted with the Wnt inhibitor topically, systemically or together with the blastocyst. Related thereto, the invention provides a method of increasing the chance of a blastocyst implantation during in vitro fertilization, comprising contacting the blastocyst with an endometrium in the presence of the Wnt inhibitor. Also provided is the use of a Wnt inhibitor for manufacturing a pharmaceutical composition for mediating blastocyst implantation during in vitro fertilization that comprises contacting the blastocyst with an endometrium in the presence of the Wnt inhibitor.

IVF includes contacting an endometrium with an embryo, the embryo could have been grown to a blastocyst stage in vitro. During growth in vitro or during or shortly after contacting the blastocyst with the endometrium, the blastocyst or the endometrium is provided with the Wnt inhibitor to increase the chance of implantation.

Also, the HIPPO pathway inhibitor as discussed improves IVF or chances of a pregnancy, similarity as discussed above with regard to the blastoid. For IVF, for increasing chances of a pregnancy or in general blastocyst preparation, said blastocyst or any preceding stage, e.g. a 1-cell stage, 2-cells stage, 4-cells stage, 8-cells stage or 16-cells stage, or morula stage, can be treated with the HIPPO pathway inhibitor. The invention provides a HIPPO pathway inhibitor for use in a method of producing a blastocyst suitable for in vitro fertilization or suitable for increasing chances of a pregnancy, comprising treating an embryo in an early stage, selected from the 1-cell stage, 2-cells stage, 4-cells stage, 8-cells stage or 16-cells stage, or morula stage or blastocyst stage until a mature blastocyst stage, with the HIPPO pathway inhibitor, especially preferred a NAEPA or a ligand of the lysophosphatidic acid (LPA) receptor, even more preferred LPA, and letting the embryo in a 1-cell stage, 2-cells stage, 4-cells stage, 8-cells stage or 16-cells stage or morula stage grow into the blastocyst stage or letting the embryo in the blastocyst stage grow into a more potent or more mature blastocyst stage. Related thereto, the invention provides a method of producing a blastocyst suitable for in vitro fertilization or suitable for increasing chances of a pregnancy, comprising treating an embryo in an early stage, selected from the 1-cell stage, 2-cells stage, 4-cells stage, 8-cells stage or 16-cells stage or morula stage or blastocyst stage until a potent or mature blastocyst stage, with the HIPPO pathway inhibitor and letting the embryo in a 1-cell stage, 2-cells stage, 4-cells stage, 8-cells stage or 16-cells stage or morula stage grow into the blastocyst stage or letting the embryo in the blastocyst stage grow into a more potent or mature blastocyst stage. Also provided is the use of a HIPPO pathway inhibitor for the manufacture of a pharmaceutical composition for producing a blastocyst suitable for in vitro fertilization or suitable for increasing chances of a pregnancy, which comprises treating an embryo in an early stage, selected from the 1-cell stage, 2-cells stage, 4-cells stage, 8-cells stage or 16-cells stage or morula stage or blastocyst stage until a potent or mature blastocyst stage, with the HIPPO pathway inhibitor and letting the embryo in a 1-cell stage, 2-cells stage, 4-cells stage, 8-cells stage or 16-cells stage or morula stage grow into the blastocyst stage or letting the embryo in the blastocyst stage grow into a more potent or mature blastocyst stage. For example, the Hippo inhibitor could be used by a patient to increase the chances of getting pregnant by improving the development of the blastocyst in utero, for example, by taking a HIPPO pathway inhibitor a few days after conception and before implantation (e.g. days 0-12, or 1-12, preferably 6 to 9). As mentioned above, Hippo inhibitor, preferably LPA or NAEPA, may be administered to a patient 1 to 12 days after conception, preferably 2 to 9 days after conception, in particular preferred 3 to 7 days after conception, to increase the chances of a pregnancy. Chances of a pregnancy are increased by promoting blastocyst development according to the invention, which may develop a higher capacity for implantation and thus development into a pregnancy. The Hippo inhibitor may be administered into the uterus.

Alternatively or in addition to the preceding paragraph, a supernatant or culture of a blastoid can be used instead or in addition to the HIPPO pathway inhibitor in this method of producing a blastocyst suitable for or during an in vitro fertilization procedure or suitable for increasing chances of a pregnancy. As has been shown in EP 2471538 A1, blastocyst culture supernatant promotes pregnancies in blastocyst transfers by producing LPA and the same is possible with a blastoid supernatant. Accordingly, the invention also provides a supernatant of a culture of a blastoid of the invention for use in a method of producing a blastocyst suitable for in vitro fertilization procedure or suitable for increasing chances of a pregnancy, comprising treating an embryo in an early stage, selected from the 1-cell stage, 2-cells stage, 4-cells stage, 8-cells stage or 16-cells stage or morula stage or blastocyst stage until a mature blastocyst stage, with the HIPPO pathway inhibitor, especially preferred a ligand of the lysophosphatidic acid (LPA) receptor, even more preferred LPA, and letting the embryo in a 1-cell stage, 2-cells stage, 4-cells stage, 8-cells stage or 16-cells stage or morula stage grow into the blastocyst stage or letting the embryo in the blastocyst stage grow into a more mature blastocyst stage. Further provided is a method of using blastoid culture supernatant to culture blastocysts intended for IVF or transferring blastoid media supernatant in uterine cavity for IVF or to increasing chances of a pregnancy, be it by IVF or increasing the chance of a natural pregnancy. A blastocyst may be implanted into said uterus and contact the blastoid media supernatant or its components in the uterine cavity. The supernatant or culture of a blastoid may be administered to a patient 1 to 12 days after conception, preferably 2 to 9 days after conception (fertilization of an egg cell), in particular preferred 3 to 7 days after conception, to increase the chances of a pregnancy. Chances of a pregnancy are increased by promoting blastocyst development according to the invention, which may develop a higher capacity for implantation and thus development into a pregnancy. The supernatant or culture of a blastoid may be administered into the uterus.

Also provided is a method of producing LPA comprising culturing a blastoid of the invention and collecting said LPA from the culture, preferably the supernatant of the culture. Since the blastoids of the invention have similar properties as the LPA-producing blastocysts described in EP 2471538 A1, the same uses are possible with the inventive blastoids as described for blastocysts in EP 2471538 A1.

The LPA produced may be any of LPA-C16:0, LPA-C16:1, LPA-C18:0, LPA-C18:1, LPA-C18:2, or a combination thereof. Any of these LPA may be used as HIPPO pathway inhibitor of the invention.

Any active agent described herein, such as the Wnt inhibitor or Hippo pathway inhibitor may be administrated, for example, (1) in vivo and systemically or (2) in vitro by exposing the embryo to the active agent before a transfer to the uterus or (3) in utero by co-transferring the molecules with the embryo upon uterus transfer. A systemic administration may e.g. be orally (e.g. a pastille, tablet, troche, lozenge, pill, gum, powder or drinking solution), parenterally (e.g. as an injection, e.g. intravenous, or as a transdermal patch). A supernatant or culture of a blastoid is preferably administered by (2) in vitro by exposing the embryo to the active agent before a transfer to the uterus or (3) in utero by co-transferring the molecules with the embryo upon uterus transfer.

The administration may be in a preparation with any one of pharmaceutical carriers, excipients, vectors, additives, or combinations thereof. The term “carrier” refers to a diluent, e.g. water, saline, excipient, or vehicle, with which the composition can be administered. For a solid or fluid composition the carriers or additives in the pharmaceutical composition may comprise SiO₂, TiO₂, a binder, such as microcrystalline cellulose, polyvinylpyrrolidone (polyvidone or povidone), gum tragacanth, gelatine, starch, lactose or lactose monohydrate, alginic acid, maize (corn) starch and the like; a lubricant or surfactant, such as magnesium stearate, or sodium lauryl sulphate; a glidant, such as colloidal silicon dioxide; a sweetening agent, such as sucrose or saccharin. Preferably, the preparation comprises buffers or pH adjusting agents, e.g. selected from citric acid, acetic acid, fumaric acid, hydrochloric ac-id, malic acid, nitric acid, phosphoric acid, propionic acid, sulfuric acid, tartaric acid, or combinations thereof.

The expression “more potent or more mature blastocyst stage” refers to an improvement in development and maturation by the HIPPO pathway inhibitor. The improvement is to a blastocyst as control or comparison that is maintained or grown under same conditions with the exception of the lack of the HIPPO pathway inhibitor used according to the invention.

The following numbered embodiments are preferred according to the invention:
1. A method of generating a blastoid or a blastocyst-like cell aggregate comprising culturing an aggregate of human pluripotent stem cells (hPSCs) and trophoblast cells in a medium comprising a HIPPO pathway inhibitor in a 3D culture.
2. The method of 1, wherein the aggregate of hPSCs and trophoblasts is generated by culturing aggregated hPSCs in a medium comprising a MEK inhibitor and a TGF-beta inhibitor, and preferably also a HIPPO pathway inhibitor.
3. The method of 2, wherein the aggregated hPSCs are formed by seeding hPSCs and aggregating the seeded hPSCs by culturing in a growth medium, preferably culturing in a growth medium for 0 to 64 hours or for 12 to 64 hours, and/or preferably wherein the growth medium comprises a ROCK inhibitor, especially preferred the ROCK inhibitor being Y27632.
4. The method of 3, wherein the seeded hPSCs have been treated before the 3D culture, preferably during a 2D culture, with a MEK inhibitor and/or a PKC inhibitor, preferably further comprising a Wnt inhibitor and/or a STAT agonist, preferably wherein treatment is in a 2D culture.
5. The method of 4, wherein the PKC inhibitor is selected from Gö6983 and Ro-31-8425.
6. The method of any one of 1 to 5, wherein the HIPPO pathway inhibitor is a ligand of the lysophosphatidic acid (LPA) receptor, preferably LPA and/or NAEPA, or verteporfin; and/or the MEK inhibitor is PD0325901; and/or the TGF-beta inhibitor is A83-1 or SB431542.
7. The method of any one of 1 to 6, wherein the medium for culturing in a 3D culture and/or the medium of culturing aggregated hPSCs as set forth in embodiment 2 further comprises a STAT3 agonist, preferably leukemia inhibitory factor (LIF).
8. The method of any one of 1 to 7, wherein the 3D culture is by culturing in a non-adherent vessel, preferably by culturing in microwells, especially preferred by microwells comprising a non-adherent surface made of hydrogel.
9. The method of any one of 3 to 8, wherein culturing of hPSCs in the growth medium of embodiment 3 comprises seeding 1 to 200 hPSCs, preferably 20 to 150 hPSCs, especially preferred 30 to 120 hPSCs, even more preferred 30 to 60 hPSCs, in a vessel and growing said seeded hPSCs in the growth medium.
10. The method of any one of 1 to 9, wherein the hPSCs and trophoblasts are cultured for at least 1 day, preferably at least 2 days.
11. The method of any one of 2 to 10, wherein the aggregated cells as set forth in embodiment 2 are cultured for at least 1 day, preferably at least 2 days.
12. The method of any one of 1 to 11 comprising culturing the cells at least until formation of a trophectoderm-like tissue, an epiblast-like tissue and a hypoblast-like tissue out of the aggregate of hPSCs and trophoblasts, preferably further at least until formation of an embryonic-abembryonic axis.
13. The method of any one of 1 to 12 comprising culturing the cells at least until formation of a three-dimensional cell aggregate with an overall diameter of at least 100 μm, preferably at least 140 μm, even more preferred 180 μm to 220 μm, formed by an outer epithelial monolayer of trophoblast-like cells surrounding a fluid-filled cavity and at least one inner cluster of cells comprising epiblast-like and hypoblast-like cells.
14. The method of any one of 1 to 13 further comprising seeding the blastoid onto endometrial cells and allowing the blastoid to implant into or onto endometrial cells.
15. The method of 14, wherein the endometrial cells have been treated with a compound selected from estrogen, estrone, estriol, ethinyl estradiol, 17α-ethylnylestradiol, mestranol, progesterone, a progestin, CAMP, and a Wnt-inhibitor, preferably XAV939, IWP-2, PNU-74654 and/or LF3.
16. The method of any one of 1 to 15 for testing or screening a candidate compound and/or candidate genetic alteration on having an effect at blastoid formation and/or implantation of a blastoid into a layer of endometrial cell comprising treating the aggregate with at least one candidate compound and/or providing the aggregate with at least one candidate genetic alteration and performing the method of any one of embodiments 1 to 15.
17. A kit suitable for culturing a blastoid, comprising a HIPPO pathway inhibitor, a MEK inhibitor, and a TGF-beta inhibitor; preferably combined in a medium for human pluripotent stem cells (hPSCs).
18. A blastoid obtainable by a method of any one of 1 to 16.
19. A blastoid comprising an outer epithelial monolayer of trophoblast-like cells surrounding at least one fluid-filled cavity and at least one inner cluster of cells comprising epiblast-like and hypoblast-like cells, wherein the outer epithelial monolayer comprises polar trophoblasts that express NR2F2.
20. An in vitro method of increasing the potential of implanting a blastoid or blastocyst into a layer of endometrial cells, comprising treating the blastoid or blastocyst with a Wnt inhibitor, preferably XAV939, IWP-2, PNU-74654 and/or LF3, and contacting the blastoid or blastocyst with the layer of endometrial cells.
21. A Wnt inhibitor for use in a method of increasing the chance of a blastocyst implantation, e.g. during in vitro fertilization or during natural pregnancy, comprising contacting the blastocyst with an endometrium in the presence of the Wnt inhibitor or stimulating the endometrial cells in the absence of a blastocyst with a Wnt inhibitor, preferably XAV939, IWP-2, PNU-74654 and/or LF3; preferably wherein the endometrium is contacted with the Wnt inhibitor topically, systemically or together with the blastocyst.
22. A HIPPO pathway inhibitor for use in a method of producing a blastocyst suitable for in vitro fertilization or suitable for increasing chances of a pregnancy, comprising treating an embryo in an early stage, selected from the 1-cell stage, 2-cells stage, 4-cells stage, 8-cells stage or 16-cells stage or morula stage or blastocyst stage until a mature blastocyst stage, with the HIPPO pathway inhibitor, especially preferred a ligand of the lysophosphatidic acid (LPA) receptor, even more preferred LPA, and letting the embryo in a 1-cell stage, 2-cells stage, 4-cells stage, 8-cells stage or 16-cells stage or morula stage grow into the blastocyst stage or letting the embryo in the blastocyst stage grow into a more mature blastocyst stage.
23. A supernatant of a culture of a blastoid of embodiment 18 or 19 for use in a method of producing a blastocyst suitable for in vitro fertilization or suitable for increasing chances of a pregnancy, comprising treating an embryo in an early stage, selected from the 1-cell stage, 2-cells stage, 4-cells stage, 8-cells stage or 16-cells stage or morula stage or blastocyst stage until a mature blastocyst stage, with the supernatant from the culture of a blastoid, and letting the embryo in a 1-cell stage, 2-cells stage, 4-cells stage, 8-cells stage or 16-cells stage or morula stage grow into the blastocyst stage or letting the embryo in the blastocyst stage grow into a more mature blastocyst stage.
24. A method of producing LPA comprising culturing a blastoid of embodiment 18 or 19 and collecting said LPA from the culture, preferably the supernatant of the culture.
25. A method of forming a trophosphere comprising culturing hPSCs in the presence of a MST1/2 inhibitor, preferably XMU-MP1, and/or a STAT inhibitor, preferably SC144.
26. The method of 25 further performed as set forth in any one of embodiments 1 to 16.
27. A method of contraception, comprising administering to a patient and/or contacting an embryo in vivo with a MST1/2 inhibitor, preferably XMU-MP-1, and/or a STAT inhibitor, preferably SC144.
28. The method of 27, wherein the MST1/2 inhibitor, preferably XMU-MP-1, and/or a STAT inhibitor, preferably SC144, is administered to a patient 1 to 9 days after conception, preferably 2 to 7 days after conception, in particular preferred 3 to 6 days after conception.
29. A MST1/2 inhibitor, preferably XMU-MP-1, and/or a STAT inhibitor, preferably SC144, for use in a method of contraception, preferably according to embodiment 27 or 28.
30. A MST1/2 inhibitor, preferably XMU-MP-1, and/or a STAT inhibitor, preferably SC144, for use in the manufacture of a contraceptive.

Throughout the present disclosure, the articles “a”, “an”, and “the” are used herein to refer to one or to more than one (i.e., to at least one) of the grammatical object of the article.

As used herein, words of approximation such as, without limitation, “about”, “substantial” or “substantially” refer to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by e.g. +10%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The “comprising” expressions when used on an element in combination with a numerical range of a certain value of that element means that the element is limited to that range while “comprising” still relates to the optional presence of other elements. E.g. the element with a range may be subject to an implicit proviso excluding the presence of that element in an amount outside of that range. As used herein, the phrase “consisting essentially of” requires the specified integer(s) or steps as well as those that do not materially affect the character or function of the claimed invention. As used herein, the closed term “consisting” is used to indicate the presence of the recited elements only.

The present invention is further illustrated by the following examples, without being limited to these embodiments of the invention.

### FIGURES

FIG. 1: Formation of human blastoids. A. Human pluripotent stem cells (hPSCs) are dissociated into single cells and seeded onto a microwell array. Under specific conditions, within 5 days, hPSCs aggregate and form a blastoid. B. The percentage of microwells including a blastoid depends on the initial number of cells that is initially seeded within the microwell.

FIG. 2: Modulation of the Hippo pathway using small molecules and genetic approaches regulates human blastoid formation. The Hippo pathway can be inhibited using Lysophosphatidic acid (LPA), NAEPA, and the YAP-TEAD complex can be suppressed using Verteporfin, which respectively increases and decreases human blastoid formation. The inhibition of the Hippo pathway can be mimicked using genetic overexpression of YAP-WT, YAP-5SA (constitutive active), which increases the formation of human blastoids. The activation of the hippo pathway can be mimicked by suppressing the formation of the YAP-TEAD complex using YAP-5SA+S94A (TEAD binding defect), which decreases the formation of human blastoids. Immunofluorescence staining of the YAP protein shows a nuclear localization only in the trophectoderm-like cells, not in the epiblast-(Nanog positive cells) and hypoblast-like cells.

FIG. 3. Evolution of the number of cells and overall aggregate size during blastoid formation. A. Upon seeding of the naïve hPSCs onto the microwell array, each microwell contains an average of 45 cells. B. After 24 hours, the cellular aggregates contain in average 45 cells that are all expressing the epiblast transcription factor Oct4. At this timepoint, cells do not express the trophoblast transcription factor GATA3. C. Between 24 and 84 hours, the number of cells increase from an average of 45 to an average of 80, and trophoblast cells expressing GATA3 appear. D. Blastoids have fully formed by 120 hours by generating analogs of the three founding cell lineages: OCT4+ epiblast-like cells, GATA4+ hypoblast-like cells, and GATA3+ trophectoderm-like cells. Similar to the human blastocyst, the average total number of cells is 120 and the most abundant lineage is trophoblast cells. E, F. Evolution of the overall size of the cellular aggregates during blastoid formation. After 24 hours, the cellular aggregates have an overall diameter of 65 micrometers. This average diameter progressively increases to 200 micrometers after 120 hours (E). Pictures of one representative aggregate stained for the nuclear dye Hoechst at 24 hours, 84 hours, and 120 hours (F). G. Once formed, human blastoids comprise analogs of the three founding lineages: An inner cluster of epiblast-like cells and hypoblast-like cells forms that is characterized by the expression of Oct4 and Nanog, and Gata4, respectively. The outer layer of the human blastoid is formed by a monolayer of trophectoderm-like cells characterized by the expression of Gata3 and Cdx2. H. Upon culture of the human blastoids for 5 days, the trophoblasts that are in contact with the inner cluster, termed polar trophoblasts, start expressing NR2F2, while the mural cells do not express it. The polar trophoblasts are known to mediate the initial attachment of the human blastocyst to the uterus. The scale bars are 25 micrometers.

FIG. 4. Evolution of the structure of the cellular aggregates during blastoid formation. A. After 24 hours, the cellular aggregates have an overall diameter of 65 micrometers and contain cells that all express the transcription factor Oct4 (see also FIG. 3B). The cells in the periphery of the aggregate express higher levels of PKC, a marker of the apical domain. B. After 84 hours, the outer cells have reinforced the formation of membrane domains expressing PKC, while the inner cells do not form such domains. The top row shows a cross section of an aggregate. The bottom row shows a full 3D projection of an aggregate. C, D. The formation of membrane domains expressing PKC coincides with the appearance of cells expressing the trophoblast transcription factor Cdx2 and with formation of small fluid-filled cavities (C) that coalesce to ultimately form a unique cavity at 120 hours (D).

FIG. 5: Formation of trophospheres by preventing the formation of the inner cluster. A. The inhibition of the STAT pathway using the small molecule SC144 or of the Hippo pathway using the small molecules XMU-MP-1 results in the formation of blastoids containing less or no inner cell cluster although trophectoderm-like cells are generated that form a cyst with a fluid-filled cavity. This points at the importance of these pathways in balancing or maintaining the number of epiblast-like cells and hypoblast-like cells. B-D. The use of 3 uM of the STAT inhibitor SC144 for 4 days results in a decrease in the formation of blastoids and an increase in the formation of trophospheres that contain either no or few epiblast-like cells and hypoblast-like cells. The use of 2 uM of the Hippo inhibitor XMUMP-1 for 4 days results in the formation of trophospheres that contain either no or few epiblast-like cells and hypoblast-like cells.

FIG. 6. Formation of an open-faced endometrial organoid and its stimulation to mimic the Window Of Implantation. A. Open-faced endometrial organoids are formed by first expanding human endometrial organoids using 3D Matrigel culture, as previously published. These human endometrial organoids are known to recapitulate molecular features of the Window Of Implantation (WOI) upon exposure to a combination of Estrogen, Progesterone and Cyclic adenosine monophosphate (CAMP). This combination is referred to as EPC. In a second step, organoids are dissociated and seeded in 2D to form an open-faced monolayer of endometrial cells. This open-faced monolayer makes the deposition of blastocyst or blastoids easy for assessing the potential to recapitulate aspects of implantation in vitro. B. Upon Wnt inhibition with either XAV939 (XAV), or (PKF118-310) PKF, the endometrial cells increase the expression of the gene PAEP, a known marker of the WOI. C. The expression of additional markers of the WOI including LIF and SPP1 are also upregulated upon Wnt inhibition and EPC stimulation as compared to EPC stimulation alone. D. Immunofluorescence shows that the open-faced endometrial organoids contain subpopulations of ciliated cells (cells positive for acetylated alpha tubulin), (E) glandular cells (FOXA2+ cells) and (F) proliferating cells (cells positive for EdU incorporation). G. The endometrial cells stimulated with EPC and Wnt inhibitors express higher levels of PAEP at the protein level as compared to non-stimulated organoids.

FIG. 7. The in vitro combination of human blastoids and open-faced endometrial organoid recapitulates features of blastocyst implantation into the uterus. Human blastoids recapitulate features of implantation into the endometrium. A, B. The interaction between human blastoids and open-faced organoids necessitate to stimulate the endometrial cells. Human blastoids fail to attach and invade into unstimulated endometrial cells that do not mimic the Window Of Implantation (WOI) (top) but attach and invade endometrial cells that are stimulated (bottom). B. The combination of EPC stimulation and Wnt inhibition using the inhibitors XAV939, IWP-2, PNU-74654, and LF3 increases the potential of endometrial cells to interact with human blastoids. C-E. The interaction between human blastoids and open-faced endometrial organoids necessitates a specific trophoblast state. The presence of the STAT inhibitor SC144 during human blastoid formation induces the formation of trophospheres with limited or no epiblast-like cells and hypoblast-like cells. The presence of the Hippo inhibitor XMU-MP-1 during human blastoid formation induces the formation of trophospheres with limited or no epiblast-like cells and hypoblast-like cells. Human trophoblast stem cells (Okae et al, 2018, doi: 10.1016/j.stem.2017.11.004) can form cytotrophoblast aggregates reflecting the post-implantation stage. SC144-trophospheres, XMU-MP-1-trophospheres and cytotrophoblast aggregates fail to attach and invade stimulated endometrial cells (C, D). On the contrary, human blastoids are capable of attaching and invading stimulated endometrial cells and attach through the polar region (D, E). F. Upon attachment and invasion, human chorionic gonadotrophin (hCGB), which is the hormone used to assess a clinical pregnancy, is detected into the culture medium after 24 and 48 hours (middle and right pregnancy test strips, respectively). Levels are undetectable if human blastoids do not attach and invade unstimulated endometrial cells (left test strip). G. Immunostaining shows that the hormone hCGB is only produced by some cells of the attached blastoids. Immunostaining also shows that the attached blastoids form numerous cells that are positive for NR2F2, a marker for polar and post-implantation trophoblasts. These trophoblasts are different from the Oct4-positive cells characteristic of the epiblast. Attached human blastoids contain trophoblasts expressing CK7, a marker of post-implantation trophoblasts. H. Upon attachment and invasion into the endometrial cells, blastoids form cells positive for the epiblast markers Oct4, Klf17, Nanog, and IFI16.

FIG. 8. Triply inhibited naive hPSCs efficiently form human blastocyst-like structures comprising analogs of the three founding lineages. a. A schematic of the time window of human peri-implantation development hereby modeled. M/MC/B=Morula/Morula Compacted/Blastocyst. b. One-step protocol of human blastoid formation. N2B27: serum-free medium. PALLY: PD0325901, A83-01, hLIF, LPA, Y-27632. c. Phase-contrast image of human blastocyst-like structures formed on a non-adherent hydrogel microwell array after 96 hours. Each microwell is 200 μm in diameter. Scale bars: 400 μm. d. Phase-contrast image of representative human blastocyst-like structures harvested from microwells. Scale bars: 200 μm (top) and 100 μm (bottom). e. Quantification of the percentage of microwells including a human blastocyst-like structure for different naive hPSC lines cultured in PALLY condition with optimized LPA concentration (yield of blastocyst-like structures (%); also see morphometric definition of a blastocyst-like structures in Methods. n=3 microwell arrays; meant S.D. f, g. Immunofluorescence stainings for the epiblast (EPI) markers (Yellow) NANOG (f) and OCT4 (g); the TE markers (Cyan) CDX2 (f) and GATA3 (g); and the primitive endoderm marker (Magenta) SOX17 (f) and GATA4 (g) in human blastocyst-like structures. Scale bar: 100 μm. h. Quantification of the absolute number of cells positive for OCT4, GATA3 and GATA4 (left) and of the ratios of cells belonging to individual lineages represented as percentage of total number of cells (right) in blastocyst-like structures (96 hours) based on immunofluorescence stainings. i. Representative immunofluorescence stainings for the tight junction molecule ZO-1 (Yellow), the adherence junction molecule CDH1 (Magenta), and the apical domain molecule aPKC (Cyan) in a representative human blastocyst-like structure. Scale bars: 50 μm.

FIG. 9. Human blastocyst-like structures form analogs of the three pre-implantation lineages. a, b. UMAP of the transcriptome of single cells originating from blastocyst-like structures (24, 60, 96 hours), naive hPSCs, primed hPSCs and hTSC (represent the post-implantation cytotrophoblast); individual cells are colored based on their origin (a) or their unsupervised cluster affiliation (b). c. Expression level of markers of each blastocyst lineage (trophectoderm (TE), epiblast (EPI) and primitive endoderm (PrE)). d. Unsupervised distance map generated using top 30 genes that are enriched in clusters 0, 1 and 3 (defined in the UMAP (see b)). e, f. UMAP of single cell transcriptome of cells from blastocyst-like structures, naive hPSCs and primed hPSCs integrated with published data sets from human embryos of pre-implantation, peri-implantation (in vitro cultured blastocysts) and gastrulation (Carnegie stage 7, i.e., between E16-19) stages. Individual cells are colored based on their origin in human embryos (e), blastocyst-like structures or stem cells (f).

FIG. 10. The three lineages form according to the sequence and time of blastocyst development. a. Schematic depicting the sequential lineage specification of human blastocysts. b. Immunofluorescence stainings for YAP1 (Yellow) with GATA2 (Cyan) in aggregates of naive hPSCs cultured in PALLY medium for 60 hours. Scale bar: 50 μm. c. Dose dependent effect of LPA on the yield of blastoids. n=3 independent microwell arrays; meant S. D.; one-way Anova and Dunnett's multiple comparisons test, ** is P=0.0016; **** is P<0.0001. d. Measurement of the effect of the overexpression of different variants of YAP1 on cavitation events in early blastoids. n=3 experiments; meant S.D.; one-way Anova and Tukey's multiple comparisons test, ns is not significant, is P=0.0004; **** is P=0.00004. e. Quantification of total cell numbers per lineage in developing blastoids at three time points of development (24, 60, 96 h). Error bar: S.D, n: Epi: 11 blastoids at 24, 68 and 96 hours; TE: 8, 14 and 15 blastoids at 24, 48 and 96 hours respectively; PrE: 9, 37 and 9 blastoids at 24, 48 and 96 hours respectively f. Immunofluorescence stainings for CDX2 (Cyan) NR2F2 (Magenta) and NANOG (Yellow) in representative B4-stage human blastocyst (left) and a blastoid (middle). Quantification of the proportion of blastoids with a preferentially polar NR2F2 expression pattern (axis) as compared to a preferentially mural NR2F2 expression pattern (inverted axis) (right). n=4 independent experiments with 4 to 12 blastoids in each experiment. meant S.D.; One-way Anova and Tukey's multiple comparisons test, * is P<0.05; *** is P<0.001. Scale bar: 50 μm. Error bar: S.D.

FIG. 11. Human blastoids recapitulate aspects of implantation. a. Schematic of implantation time modeled (left). Open Face Endometrial Layer (OFEL) primed for receptivity with EPC/XAV939 as an implantation assay (right). E2: Beta-estradiol. EPC: E2, Progesteron, CAMP. b. Representative phase contrast images of blastoids (GFP⁺) 24 hours after deposition onto non-stimulated (top left) or stimulated OFELs (bottom left). Scale bar: 100 μm. Attachment efficiency of human blastoids (right). n=7 independent experiments from 3 different donors; meant S.D.; Unpaired two-tailed t-test, **** is P=4.5e-8. c. Representative images of recently attached human blastoids (12±4 hours). Dotted line outlines the inner cluster of blastoids formed from GFP⁺ naive hPSCs (top). Scale bar: 100 μm. X-Z plane of immunofluorescence stainings for NR2F2 (Magenta) and OCT4 (Yellow) in blastoids immediately after attachment (bottom). Scale bar: 5 μm. d. Intensity profile of immunofluorescence stainings (NR2F2, OCT4) in blastoids immediately after attachment. n=10. e. Representative phase contrast images of trophospheres formed using 3 UM SC144 (top) or 2 μM XMU-MP-1 (middle), and aggregates of hTSCs (bottom) deposited onto stimulated OFELs. Scale bar: 100 μm. Attachment efficiency (right). n=3 independent experiments; meant S.D.; one-way Anova and Dunnett's multiple comparisons test, **** is P<0.0001. f. Pregnancy test strips detecting human chorionic gonadotropin (CGβ) secretion into the medium of unstimulated OFELs with unattached blastoids and stimulated OFELS with attached blastoids (48 hours on OFEL, also see ELISA assay in FIG. 21b). g. Immunofluorescence stainings for OCT4 (Yellow) and aPKC (Grey) in human blastocysts (left) or blastoids (right) grown in postimplantation culture condition for 4 days. Counterstain with Phalloidin marking F-actin (Cyan). Scale bar: 100 μm. h. Number of cells positive for OCT4, GATA3, and GATA4 in blastoids grown in postimplantation culture condition for 6 days (time equivalent=day 13). n=5. meant S.D.

FIG. 12. Naive hPSCs form human blastocyst-like structures comprising analogs of the three founding lineages. a. Phase contrast images of naive hPSCs cultured in PXGL medium and on MEF feeder layers. Scale bar: 50 μm. b. Time course phase contrast images of naive hPSCs aggregates cultured within microwell arrays either without LPA (PALY medium, top) or with 500 nM LPA (PALLY medium, bottom). Scale bar: 200 μm. c. Quantification of the effect of the initial cell numbers per microwell array on the yield of blastocyst-like structures. n=1 microwell arrays. d. Quantification of the effect of serial passaging of naive hPSCs on the yield of blastocyst-like structures. n=3 microwell arrays. meant S.D. e. Quantification of the cell numbers per microwell at the time of seeding and in blastocyst-like structures at 96 hours when cells are seeded at 3.0×10⁴ cells per microwell array. n=190 microwells (seeding) and n=12 blastocyst-like structures (96 hrs.). f. Fluorescence staining of DNA using Hoechst in representative naive hPSCs aggregates over the course of formation of blastocyst-like structures (96 hours, left). Scale bar: 50 μm. Measurement of the distributed diameters of the structures over the course of formation of blastocyst-like structures (right). n=15, 31 and 11 for 0, 60 and 96 hours, respectively. g. Pseudotime analysis of human pre-implantation development showing the expression of the TE markers GATA2, GATA3, CDX2 and TACSTD2. Gene expression analysis was performed by using the public data analysis tool (https://bird2cluster.univ-nantes.fr/demo/PseudoTimeUI/). h. Immunofluorescence stainings for EPI marker NANOG (Yellow), TE marker CDX2 (Cyan) and primitive endoderm marker GATA4 (Magenta) in a representative B4-stage human blastocyst. Scale bar: 50 μm. i. Immunofluorescence stainings for the EPI markers (Yellow) NANOG (top) and OCT4 (bottom); the TE markers (Cyan) CDX2 (top) and GATA3 (bottom); and the primitive endoderm marker (Magenta) GATA4 in five representative blastocyst-like structures. Counterstain with Hoechst (Grey) marking DNA. Scale bar: 50 μm. j. Immunofluorescence staining for EPI marker OCT4 (yellow) and TE marker GATA2 (Cyan) in blastocyst-like structures. Scale bar: 100 μm. k. Immunofluorescence staining for TE markers GATA3 (Cyan) and TROP2 (Magenta) in blastocyst-like structures. Scale bar: 100 μm. l. Immunofluorescence staining for TE markers GATA3 (Cyan) and GATA4 (Magenta) and the PrE marker PDGFRa (Yellow) in blastocyst-like structures. Scale bar: 100 μm. m. Single optical section of immunofluorescence staining image for the tight junction molecule ZO-1 (Yellow), the adherence junction molecule CDH1 (Magenta), and the apical domain molecule aPKC (Cyan) in a representative human blastocyst-like structures. Scale bars: 50 μm. n. Representative time points from a timelapse image of naive cell aggregates, cavitating into blastocyst-like structures while showing cycles of cavity inflation and deflation (left)quantification of blastocyst-like structures showing distinct frequencies of inflation and deflation (right). n=1 microwell arrays. Scale bar: 100 μm. o. Phase contrast images of representative areas of microwell arrays showing blastocyst-like structures formed from different naive hPSCs and hiPSCs lines. n>3. Scale bar: 100 μm. p. Quantification of the yield of blastocyst-like structures obtained from naive and primed H9 hPSCs. n=3 microwell arrays. meant S. D.

FIG. 13. Human blastocyst-like structures form analogs of pre-implantation lineages. a. Flow cytometry analysis plot of cells isolated from blastocyst-like structures and stained for lineage-specific surface markers PDGFRa (PrE) and TROP2 (TE). The gates were used to sort analogs of EPI (double negative), TE (TROP2^{high}) and PrE (PDGFR+^{high}) to subsequently process for single cell RNA sequencing. Note that the gates did not exclude any cells. This analysis was performed to correlate RNA measures, while ensuring a representation of all cell types. b. UMAPs of the transcriptome of single cells isolated from blastocyst-like structures and displaying the expression levels of genes specific for each of the three blastocyst lineages (TE—Trophectoderm, EPI—Epiblast, and PrE—Primitive endoderm). c-g. UMAPs of single cells isolated from both blastocyst-like structures and from embryos ranging from E3 to E19. c. Coloration of cells originating from In Vitro Fertilization (IVF) embryos isolated on day 3 (E3) to day 7 (E7). This period comprises only preimplantation stage embryos. d. Coloration of cells originating from IVF embryos isolated on day 6 (E6) to day 12 (E12). These blastocysts (E6) were cultured in vitro. Note that this annotation reflects the number of days in culture rather than the developmental stages. e. Coloration of cells originating from gastrulation-stage embryo isolated on day 17 (E17) to 19 (E19). f. The expression levels of genes specific for each of the three blastocyst lineages (EPI, TE, and PrE). g. Coloration of cells displaying their unsupervised cluster affiliation.

FIG. 14. Measurement of generation of off-target cells in human blastocyst-like structures and naive human pluripotent stem cells. a,b. UMAP of clusters formed from cells isolated from blastocyst-like structures (high-resolution clustering of 1, ×50 as compared to FIG. 2b) (a) and displaying the expression levels of genes specific for amnion lineage (b). c. Origin of the cells composing cluster 11. d-h. UMAPs of naive hPSCs, primed hPSCs, cells isolated from blastocyst-like structures and cells isolated from a CS7 staged human embryo. d. Coloration of embryo cells. e. Coloration of stem cells based on their origin. f. Display of the expression levels of genes specific for each of the three blastocyst lineages (EPI—Epiblast, TE—Trophectoderm, and PrE—Primitive endoderm). g. Coloration of individual cells based on their unsupervised cluster affiliation. h. Coloration of the cells previously identified as cluster 11 (see a, b). i. Quantification of the percentage of cells identified as abnormal based on the location in the UMAP in h (top) and on the cells annotations (bottom) for both naive hPSCs (left) and cells isolated from blastocyst-like structures (right). Similar results were obtained based on the location in the UMAP in (FIG. 13c-e). j. Heatmap of markers of different lineages differentially expressed in cells from blastocyst like structures and gastrulation-stage embryo.

FIG. 15. Cells in human blastocyst-like structures are transcriptionally similar to pre-implantation lineages. a. Principal component analysis (PCA) plot with PC1 vs PC2 (top) or PC1 vs PC3 (bottom) computed with top 500 variable gene in the bulk transcriptome of individual lineages of blastocyst like structures (EPI, TE and PrE); stem cell lines: naive and primed hPSCs; hTSCs: blastocyst derived hTSCs (bTS5), primed hPSC derived hTSCs (BAP and TM4 protocols; PrE like stem cell lines (RACL or nEND cells); naive PSC and TSCs rederived from blastocyst-like structures (see extended methods). b. Heatmap of key blastocyst and post-implantation lineage markers differentially expressed between TE analogs (TROP2+) of the blastocyst-like structures and hTSCs in their bulk transcriptome. c. Pseudotime analysis of human mature TE markers CGB5, CGB7, CGB8 and CGA. Gene expression analysis was performed by using the public data analysis tool (https://bird2cluster.univ-nantes.fr/demo/PseudoTimeUI/). d. Heatmap of key pluripotency related genes differentially expressed between EPI analogs (PDGFR⁻/TROP2⁻) in the blastocyst-like structures and primed hPSCs. e. Heatmap of key pluripotency related genes or PrE markers differentially expressed between PrE analogs (PDGFRα⁺) in the blastocyst-like structures, naive PSC derived PrE-like cells and nEND cells.

FIG. 16. Human blastocyst-like structures are permissive for derivation of stem cell lines. a. Immunofluorescence staining for pluripotency factors NANOG (Yellow), OCT4 (Magenta), SOX2 (Cyan) and for naive pluripotency factor KLF17 (Yellow) in naive hPSC controls (top) and naive hPSCs derived from blastocyst-like structures (bottom). Scale bar: 100 μm. b. Phase contrast images of blastocyst-like structures on microwell array formed from three rederived naive hPSC lines. Scale bar: 200 μm. c. Immunofluorescence stainings for EPI marker (NANOG), TE marker (CDX2) and primitive endoderm marker (GATA4) in representative second-generation blastocyst-like structures. Scale bar: 100 μm. d. Immunofluorescence staining for GATA3 (Cyan), post-implantation trophoblast marker CK7 (Magenta) and CDX2 (Yellow) in bTS5 hTSC (top) and hTSCs derived from blastocyst-like structures (bottom). Scale bar: 100 μm. e. Phase contrast images of day 6 EVT differentiations from three hTSC lines derived from blastocyst-like structures. Scale bar: 150 μm. f. Immunofluorescence stainings of trophoblast markers GATA3 (Cyan) and EVT marker HLA-G (Yellow) and CGB (Magenta) of day 6 EVT analogs from three hTSC lines, derived from blastocyst-like structures. Scale bar: 100 μm. g. Phase contrast images of day 3 SCT analogs differentiated from three hTSC lines derived from blastocyst-like structures. Scale bar: 150 μm. h. Immunofluorescence stainings for trophoblast markers GATA3 (Cyan) and SCT marker SDC1 (Yellow) and CGB (Magenta) of day 3 SCT analogs formed from hTSC line derived from blastocyst-like structure (Clone 1). Scale bar: 100 μm. i. Immunofluorescence stainings for CGB (Magenta) counterstained with Phalloidin (Cyan) and Hoechst marking Actin and DNA respectively (left), SDC (Yellow), CK7 (Magenta) (right) counterstained with Hoechst marking DNA of day 6 trophoblast organoids formed from hTSC lines derived from blastocyst-like structures (Clone 1). Scale bar: 50 μm. j. Relative expression levels, as measured by RT-PCR, of day 6 EVT (top) and day 3 SCT analogs (bottom) with respective undifferentiated hTSCs lines derived from blastocyst-like structures. Expression levels were normalized to expression of GAPDH. n=1 biological replicate for three individual clones.

FIG. 17. The development of the human trophectoderm analog depends on aPKC and Hippo elements. a. A frame from time-lapse microscopy of B2 stage human blastocyst (left). Schematic showing the differential Hippo activity in inner and outer cells of developing blastocyst and the molecular regulators of the Hippo signalling pathway (right). b. Phalloidin fluorescence (Cyan) stainings for F-actin in naive hPSCs aggregates cultured in PALLY medium for 24 hours (top) and 60 hours (bottom). Counterstain with Hoechst marking DNA. Scale bar: 50 μm. c. Immunofluorescence stainings for aPKC (Cyan) and YAP1 (Yellow) in aggregates of naive hPSCs cultured in PALLY medium for 24 hours (top) and 60 hours (bottom). Counterstain with Hoechst marking DNA. Scale bar: 50 μm. d. Immunofluorescence stainings for YAP1 (Yellow) with GATA2 (Cyan) in aggregates of naive hPSCs cultured in PALLY medium for 24 hours. Scale bar: 50 μm. e. Immunofluorescence stainings for YAP1 (Yellow) and GATA3 (Cyan) (top) and YAP1 (Yellow) and NANOG (Cyan) (bottom) in naive hPSCs aggregates cultured in PALLY medium for 24 hours (left) and 60 hours (right). Counterstain with Hoechst marking DNA. Scale bar: 50 μm. f. Immunofluorescence staining for YAP1 (Yellow) and GATA3 (Cyan) in blastoids cultured without (top) or with an aPKC inhibitor (2 μM CRT0103390, bottom). Counterstain with Hoechst marking DNA (Red). Insets: Individual and merge channels of YAP1 and GATA3 for a single optical section as well as maximum intensity projection of all the optical sections. Scale bar: 50 μm. g. Quantification of the yield of blastoids upon the culture in PALLY medium or PALLY medium complemented with an aPKC inhibitor (2 μM CRT0103390). n=3 independent microwell arrays; meant S. D.; Two tailed unpaired t-test. *** is P=0.0002. h. Quantification of the percentage of GATA3+ cells in structures cultured in PALLY medium or in PALLY medium complemented with a aPKC inhibitor (2 UM CRT0103390). n=7 blastoids for the group cultured in PALLY medium and n=12 aggregates for the group cultured in PALLY medium complemented with CRT0103390. Representative results from three independent experiments. Meant S. D.; Two-tailed unpaired t-test. **** is P=1.79e-08. i. Quantification of the dose dependent effect of the LPA receptor agonist NAEPA on the yield of blastoids. The PALY medium (thus without LPA) was complemented with NAEPA. n=3 independent microwell arrays; meant S. D.; one-way Anova and Tukey's multiple comparisons test. ** is P<0.0001. j. Phase contrast images of representative naive hPSC aggregates cultured in PALLY medium complemented with Doxycycline (100 ng/ml) for 72 hours and overexpressing different variants of YAP1. The naive hPSCs aggregates were cultured with an adjusted PALLY medium characterized by a reduced LPA concentration (5 nM). Scale bar: 100 μm. k. Measurement of the effect of Verteporfin (suppressor of the YAP1-TEAD complex) on the yield of blastoids. n=3 independent microwell arrays; meant S.D.; one-way Anova and and Dunnett's multiple comparisons test. ** is p=0.0010, *** is p=0.00019, **** is P<0.0001. l. Phalloidin fluorescence staining of F-actin (Cyan) in naive hPSCs aggregates cultured in PALLY medium for 60 hours. Counterstain with Hoechst marking DNA. Yellow arrows: Formation of cavities. Scale bar: 50 μm. m. Immunofluorescence stainings for Aquaporin3 (AQP3, Cyan) and OCT4 (Yellow) in naive hPSCs aggregates cultured in PALLY medium for 36 (left) or 96 hours (right, blastoid stage). Scale bar: 50 μm.

FIG. 18. Blastoids recapitulate the sequential specification of lineages occurring during blastocyst development. a. Heatmap of the average count values in the expression of TE genes upon formation of the blastoid TE analogs. b-d. Immunofluorescence stainings for GATA3 (Cyan) and OCT4 (Yellow) (b) or CDX2 (Cyan) and NANOG (Yellow) (c) or CDX2 (Cyan) and KLF17 (Yellow) (d) in naive hPSCs aggregates cultured in PALLY medium for 24 hours (top) or 60 hours (bottom). Scale bar: 50 μm. e. Gene ontology terms associated with the genes, differentially regulated in the late TE analog of blastoids (cluster 10) as compared to the early TE (cluster 2). f. Heatmap of average count values of Wnt, TGF-β and Notch signaling-associated genes in cells from cluster 4 (naive hPSCs), 10, 2 and 5 (TE analogs) and 7 (TSC). g. UMAPs of single cells isolated from blastoids and displaying the expression levels of polar trophectoderm specific gene: NR2F2. h. Immunofluorescence staining for CDX2 (Cyan), NR2F2 (Magenta) and NANOG (Yellow) in blastoids. Scale bar: 100 μm. i. UMAPs of single cells isolated from blastoids and displaying the expression levels of polar trophectoderm specific gene: CCR7. j. Immunofluorescence stainings for CCR7 (Cyan) in a blastoid. Counterstain with Hoechst marking DNA. Scale bar: 50 μm. k. Heatmap of average count values of top differentially regulated genes in cells from cluster 4 (naive hPSCs), 0 (EPI analogs) and 9 (primed hPSCs). 1. Immunofluorescence staining for KLF17 (Cyan) and OCT4 (Yellow) or KLF4 (Cyan) and OCT4 (Yellow) (top) and SUSD2 (Cyan) and NANOG (Yellow) or IFI16 (Cyan) and KLF17 (Yellow) (bottom) in blastoids. Counterstain with Hoechst marking DNA. Scale bar: 100 μm. m. UMAPs of single cells isolated from blastoids and displaying the expression levels of X chromosome activation-related gene-XACT. n. Flow cytometry analysis plot of cells isolated from blastocyst-like structures cultured in PALLY medium for 60 hours and stained for lineage-specific surface markers PDGFRa (PrE) and TROP2 (TE). o, p. Immunofluorescence stainings for OTX2 (Cyan), GATA4 (Magenta) and OCT4 (Yellow) (o) and SOX17 (Cyan) and GATA4 (Magenta) (p) in naive hPSCs aggregates cultured in PALLY medium for 60 hours. Counterstain with Hoechst marking DNA. Scale bar: 50 μm. q. Heatmap of the average count values in the expression of PrE genes upon formation of the blastoid PrE analogs. r. Heatmap of average count values of SMAD, MAPK and Wnt signaling-associated genes in cells from cluster 1, 6 (EPI analogs) and 8 (PrE analogs).

FIG. 19: Human blastoids recapitulate aspects of implantation. a. Immunofluorescence stainings for CDH1 (Magenta) and a ciliated cell marker acetylated α-tubulin (Yellow) in OFELs (left). Y-Z plane shows the apical location of the cilia (right). Scale bar: 50 μm. b. Immunofluorescence staining for FOXA2 (Yellow) marking the endometrial glandular cells in OFELS. Scale bar: 50 μm. c. Immunofluorescence staining for PAEP (Yellow) in non-stimulated (left) and stimulated (right) OFELs. d. qRT-PCR measurement of the expression levels of window-of-implantation markers in OFELs cultured with different media. Ctrl: Control medium, E: Estradiol, P: Progesterone, C: CAMP, X: XAV939. Expression levels were normalized relative to the housekeeping gene GAPDH and the control condition. n=2 independent experiments. The colors depict the data from 3 different donors. e. Heatmap of key cell cycle and secretory epithelial genes differentially expressed between stimulated and non-stimulated OFELs in bulk transcriptome. f. Staining for incorporated EdU (Red) reflective of cell proliferation in a stimulated OFEL (left). Scale bar: 50 μm. Quantification of the number of EdU⁺ cells in non-stimulated and stimulated OFELS (right). Counterstain with Hoechst marking DNA. n=4 independent experiments. meant S. D.; Unpaired two-tailed t-test, *** is P=0.0009. g. Quantification of blastoid attachment onto OFELs prepared using endometrial organoids from 3 different donors. n=3 independent experiments for donor 1 and n=2 independent experiments for donor 2 and 3; meant S.D.; Unpaired two-tailed t-test, ** is P=0.0011. h. Immunofluorescence stainings for MUC1 (Magenta), a glycoprotein that highly expresses at the luminal epithelial surface of endometrium in the receptive phase, with an attached GFP+ blastoid (48 hours after deposition onto an OFEL). Dashed lines indicate the area that trophoblast cells repelled endometrial cells. Scale bar: 200 μm i. Quantification of blastoid attachment onto non-stimulated, stimulated OFELs, and OFELs additionally exposed to the contraceptive Levonorgestrel (LNG, 10 μM). n=3 independent experiments. meant S. D.; one-way Anova and Tukey's multiple comparisons test, * is P=0.0211, *** is P=0.0006.

FIG. 20: Trophectoderm state is crucial for interaction with endometrium during implantation. a. Representative images of human blastoids shortly after attachment to an OFEL. Dotted line outlines the inner cluster of blastoids that were formed using GFP⁺ naive hPSCs (top). Immunofluorescence stainings for NR2F2 (Magenta) and OCT4 (Yellow) in blastoids shortly after attachment to an OFEL (bottom). b. Immunofluorescence stainings for NR2F2 (Magenta) and OCT4 (Yellow) and respective fluorescence intensity profiles of representative blastoids immediately after attachment onto OFEL. Profiles were measured perpendicular to the plane of attachment (right). Line width, 10 μm. Y axis shows normalized intensity. c. Quantification of the distance between the first peak of fluorescence intensity profiles of NR2F2 and OCT4. n=10 attached blastoids. meant S.D. d. Pseudotime analysis of human pre-implantation development showing the expression of IL6, IL6R, GP130 and STAT3. Gene expression analysis is performed by using the public data analysis tool (https://bird2cluster.univ-nantes.fr/demo/PseudoTimeUI/). e. Quantification of the dose dependent effect of LIF on the yield of blastoids. n=2 (without Lif) and n=3 (all other conditions) independent experiments. mean±S.D. f. Immunofluorescence staining for NANOG (Yellow) and CDX2 (Cyan) (left), OCT4 (Yellow) and GATA3 (Cyan) (middle) and CDX2 (Cyan) and NR2F2 (Magenta) (right) in representative trophospheres formed from a blastoid exposed to SC144. Scale bar: 50 μm. g. Immunofluorescence staining for NANOG (Yellow) and CDX2 (Cyan) (left), OCT4 (Yellow) and GATA3 (Cyan) (right) in representative trophospheres formed from a blastoid exposed to XMU-MP-1. Scale bar: 50 μm. h. Heatmap of key lineage specific genes differentially expressed in bulk transcriptome of the trophectoderm of blastoids (TROP2 positive cells), trophospheres (SC144 or XMU) and TSCs (2D or 3D) compared to naive hPSCs. i. PCA plot computed using bulk transcriptome of blastoid cells, hPSCs (naive, primed or blastoid rederived naive cell lines), TSCs (bTS5, blastocyst rederived lines or human stem cell derived TSC like cells) and pluripotent stem cell derived primitive endoderm like cells (RACL or NACL cells). j. Immunofluorescence stainings for CDX2 (Cyan) (left) and CK7 (Magenta) and GATA3 (Cyan) (right) in aggregates formed from bTS5 hTSCs. Counterstain with Hoechst marking DNA. Scale bar: 50 μm. k. Representative phase contrast images of aggregates of naive hPSCs, deposited onto stimulated OFELs. Scale bar: 100 μm. l. List of selected putative ligand-receptor pairs involved in cross-talk between polar trophectoderm and endometrial epithelial cells. The list was generated by in silico ligand receptor analysis of genes enriched in polar trophectoderm and stimulated OFEL, using Cellinker.

FIG. 21. Human blastoids recapitulate aspects of peri-implantation progression until day 13. a. Bright-field images of human blastoids (96 hours) cultured for 4 additional days on a low attachment plate in post implantation culture condition (left). Each row shows a time series of an individual blastoid for 4 days. Note that, blastoids stably retain cavities at least for 2 days upon transferring to IVC media which has different osmolarity compared to the N2B27 media with PALLY. (See the extended methods for the composition of post implantation culture media.) Scale bar: 200 μm. Quantification of percentage of blastoids retaining cavities on each day of postimplantation stage culture (right). n=2 independent experiments. b. Immunofluorescence staining for the syncytiotrophoblast-associated marker CGB (Magenta) in GFP⁺ blastoids attached onto stimulated OFELS (48 hours after deposition) (left). Counterstain with Hoechst marking DNA. Scale bar: 50 μm. ELISA measurements of the concentration of the protein CGB secreted into the culture medium of unstimulated OFELs with unattached blastoids and stimulated OFELS with attached blastoids (24 and 48 hours) (right). n=3 independent experiments. meant S.D.; one-way Anova and Tukey's multiple comparisons test, ** is P=0.00006. c. Immunofluorescence stainings for CDX2 (Cyan), NR2F2 (Magenta) and SOX2 (Yellow) in blastoids grown in postimplantation culture condition for 4 days. Scale bar: 100 μm. d. Immunofluorescence stainings for OCT4 (Yellow), CK7 (Cyan) and GATA4 (Magenta) in blastoids grown in postimplantation culture condition for 4 days. Scale bar: 100 μm. e, f. Immunofluorescence stainings for CGB (Magenta) and NR2F2 (Cyan) (e) or HLA-G (Magenta) and GATA3 (Cyan) (f), in blastoids grown in postimplantation culture condition for 4 days (e) or 6 days (f). Counterstain with Hoechst marking DNA. Arrowhead points HLA-G positive EVT like cells. Scale bar: 100 μm. g. Immunofluorescence stainings for CD24 (Magenta) and SOX2 (Yellow) in blastoids grown in postimplantation culture condition for 6 days. Counterstain with Hoechst marking DNA. Scale bar: 100 μm. h. Immunofluorescence stainings for PODXL (Magenta) and SOX2 (Yellow) in blastoids grown in postimplantation culture condition for 4 days. Counterstain with Phalloidin marking F-actin (Cyan). Arrowhead points pro-amniotic-like cavity. Scale bar: 100 μm. i-k. Immunofluorescence stainings for SOX2 (Yellow), GATA3 (Cyan) and CDX2 (Magenta) (i), SOX2 (Yellow), CDX2 (Magenta) and TFAP2C (Cyan) (j), OCT4 (Yellow), GATA4 (Magenta) and OTX2 (Cyan) (k) in blastoids grown in postimplantation culture condition for 4 days. Counterstain with Hoechst marking DNA. Scale bar: 100 μm. 1. Quantification of number of cells belonging to EPI, TE or PrE lineages in the blastoids cultured in postimplantation culture condition for four days on glass or OFEL. n=7 biological replicates. meant S.D. m. Immunofluorescence stainings for OCT4 (Yellow), GATA3 (Cyan) and GATA4 (Magenta) in blastoids grown in postimplantation culture condition for 6 days corresponding to time equivalent of day 13 of cultured human blastocyst (left). Scale bar: 100 μm.

FIG. 22. Quantification of the percentage of microwells including a human blastocyst-like structure formed from aggregated naive hPSCs stimulated or not stimulated with LPA and PD0325901 and A83-01 (triple inhibition).

FIG. 23. Phase contrast images of representative areas of microwell arrays showing blastoids formed by the 4 days treatment of two different TGFb signaling inhibitors 1 μM A83-01 (top) or 1 μM SB431542 (bottom, abbreviated “SB43”) in combination with PLLY. Quantification of the yield of blastoid structures. The dotted line represents the yield of blastoid with a standard protocol (PALLY for 2 days and LY for 2 days). n=3 microwell arrays. Error bar: S. D. PLLY: 1 UM PD0325901, 500 nM LPA, 10 ng/ml hLIF, 10 PM Y-27632.

### EXAMPLES

### Example 1: Formation of an Aggregate of hPSCs

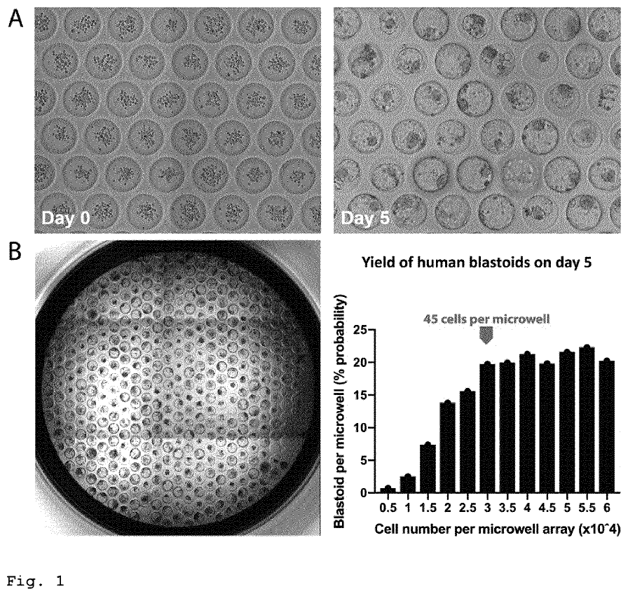

The formation of blastoids relies on the aggregation of an optimal number of hPSCs. This can be achieved by seeding specific numbers of hPSCs onto a microwell array made of non-adherent hydrogel and containing numerous microwells of, for example, 200 micrometers (FIG. 1A). We observed that human blastoids preferentially form from aggregates of 20-100 cells, which favors the formation of aggregates capable of forming a cavity (FIG. 1B). The phenomena is quantified by measuring the number of structures that form a cavity, and of blastoids defined as a three-dimensional structure with an overall diameter comprised between 180 and 220 micrometers, formed by an epithelial monolayer of trophoblast-like cells surrounding a unique fluid-filled cavity and (an) inner cluster(s) of cells composed of epiblast- and hypoblast-like cells. The timing of molecular stimulation of the aggregate also determines the level of cellular specification and self-organization within this aggregate. We observed that the initial aggregation of the cells in B27N2 medium, followed, 24-48 hours later, by the replacement of the culture medium by a B27N2 medium containing A83-1, PD0325901, LIF is sufficient for the aggregate to form the three cell types and the outer trophoblast cyst that contains a single inner cell cluster of epiblast and hypoblast cells. Under these conditions, human blastoids form within 5 days (FIG. 1B).

### Example 2: Inhibition of the HIPPO Pathway by the Use of Either Small Molecules or Genetic Approaches

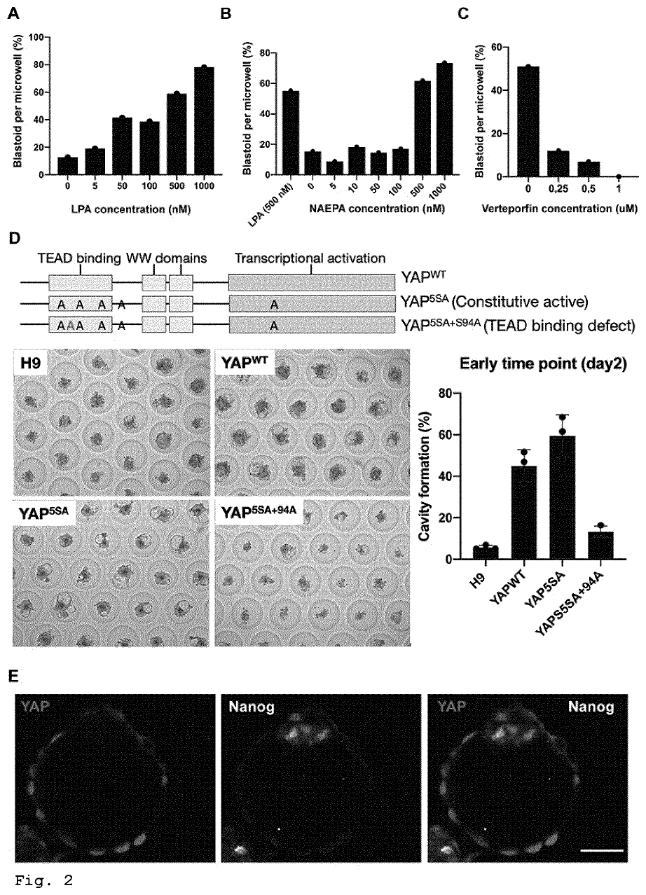

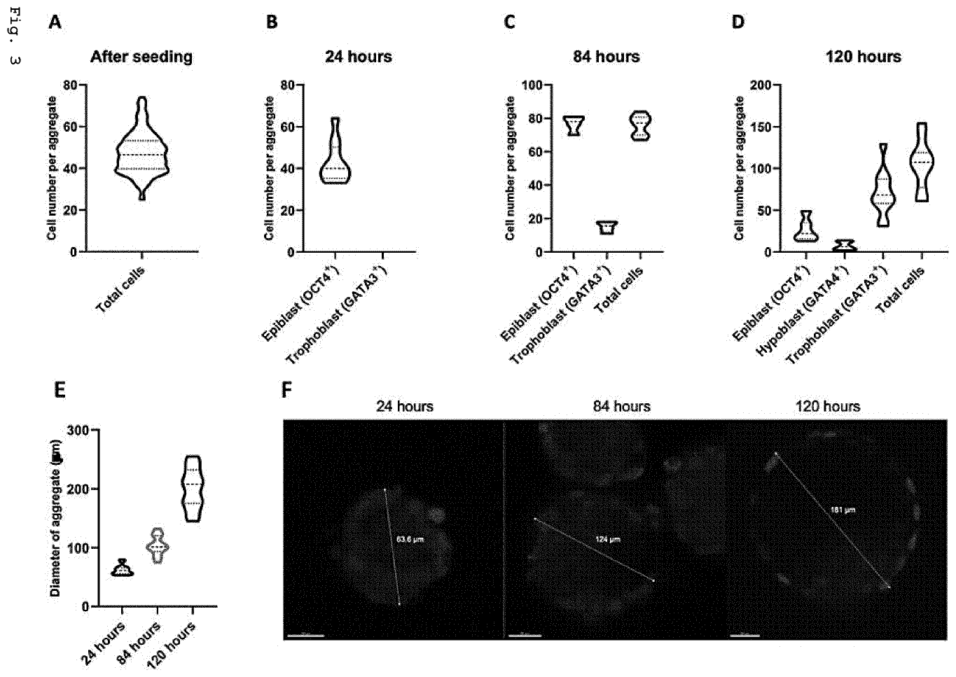

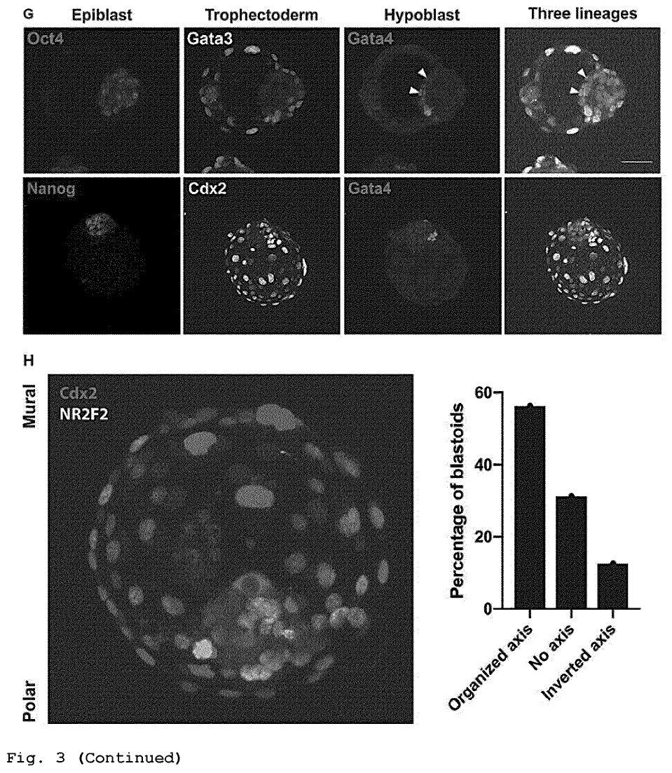

The activity of the HIPPO pathway is essential for blastoid formation. The stimulation of hPSCs aggregates using the small molecule LPA or NAEPA, inhibitors of the HIPPO pathway activity, leads to the transition from a solid aggregate of cells to a cystic structure including a single fluid-filled cavity. This fluid-filled cavity is lined by cells expressing trophectoderm markers, including CDX2 and GATA3, and comprises a single cluster of cells expressing markers of the epiblast, including NANOG and OCT4, and cells expressing markers of the hypoblast, including GATA4. The necessity of modulating the activity of the HIPPO pathway to form blastoids is also assessed by the effect observed upon treatment of the aggregate of hPSCs with the YAP-specific inhibitor, Verteporfin. This small molecule totally prevents the formation of a cavity (1 μM) (FIG. 2C). In accordance with the role of the HIPPO pathway in human blastoid formation, the HIPPO pathway effector YAP is translocated into the nuclei of the outer trophectoderm-like cells, while it is not located in the nuclei of the inner cell cluster (FIG. 2E). Finally, overexpression of wild-type and constitutive active YAP (YAP 5SA) facilitates the cavity formation in the early stage of blastoid formation (Day 2) (FIG. 2D). On the contrary, overexpression of YAP with the mutation in the TEAD binding site (YAP S94A), which is known to prevent the activation of the HIPPO target genes, does not show any positive effect on cavity formation. Thus, consistent with the data generated using small molecules, the modulation of the HIPPO pathway is necessary for the genetic activation of the trophectoderm program and the formation of a blastocoel-like cavity.

### Example 3: Concomitant Specification and Morphogenesis of Trophectoderm-Like, Epiblast-Like, and Hypoblast-Like Tissues

Inhibition of LIF-STAT pathway by treatment of chemical inhibitor SC-144 induced the formation of mono-layer cavitated aggregate that does not form an inner cluster of Epi-/Hypo-like cells. The maturation of lineage specification of outer trophectoderm cells were also compromised by the SC-144 treatment, which indicates the crucial role of cell-cell communication among three lineages for blastoid formation.

### Example 4: Spontaneous Formation of the Embryonic-Abembryonic Axis

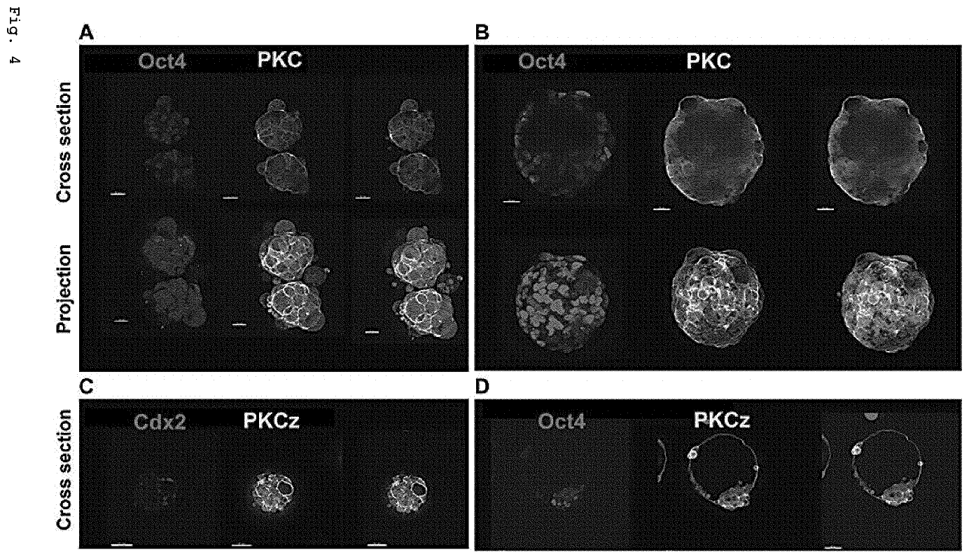

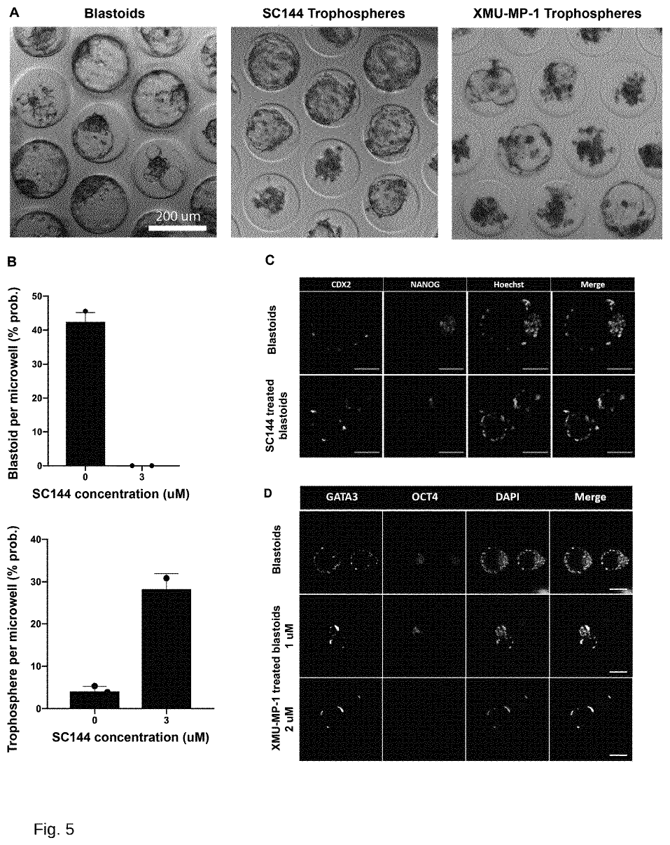

In the late human blastocyst, the polar trophectoderm that juxtapose the epiblast starts expressing the transcription factor NR2F2, thus marking the region that will mediate the implantation into the uterus. Similarly, upon in vitro culture the trophectoderm-like tissues of blastoids spontaneously form a distinct region characterized by the expression of NR2F2. On the contrary, the mural trophectoderm opposite the fluid-filled cavity does not express NR2F2 (FIG. 3H). Thus, similar to blastocyst, blastoids are capable of spontaneously forming the region that mediate the implantation, and of forming an axis.

### Example 5: Differentiation of Endometrial Cells into Receptive Cells Mimicking the Window of Implantation

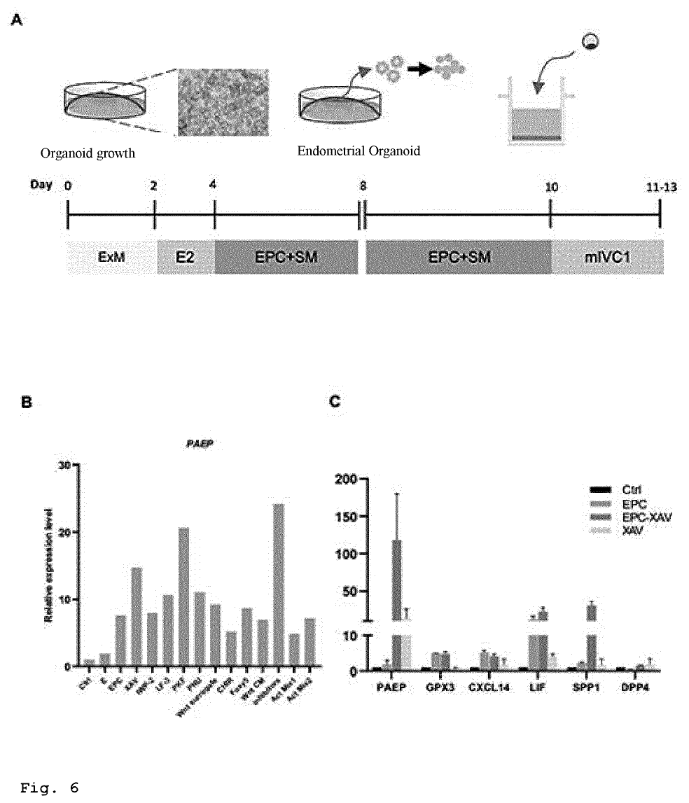

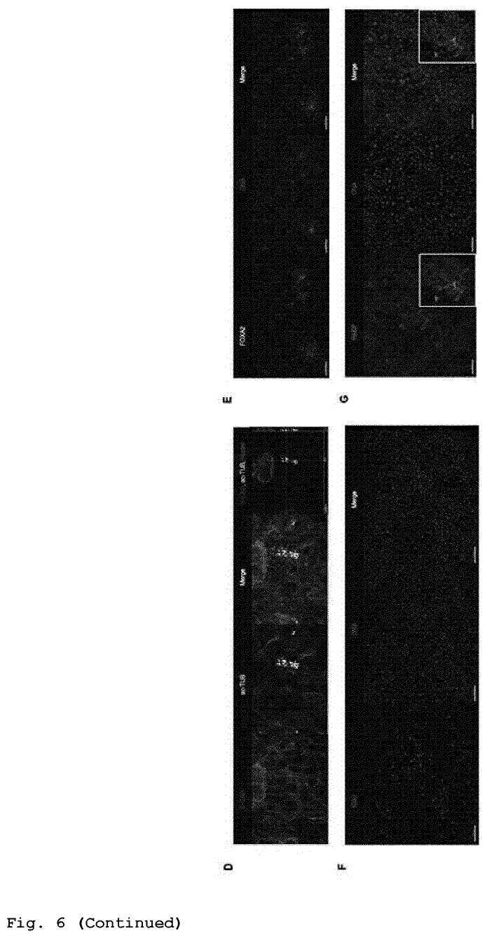

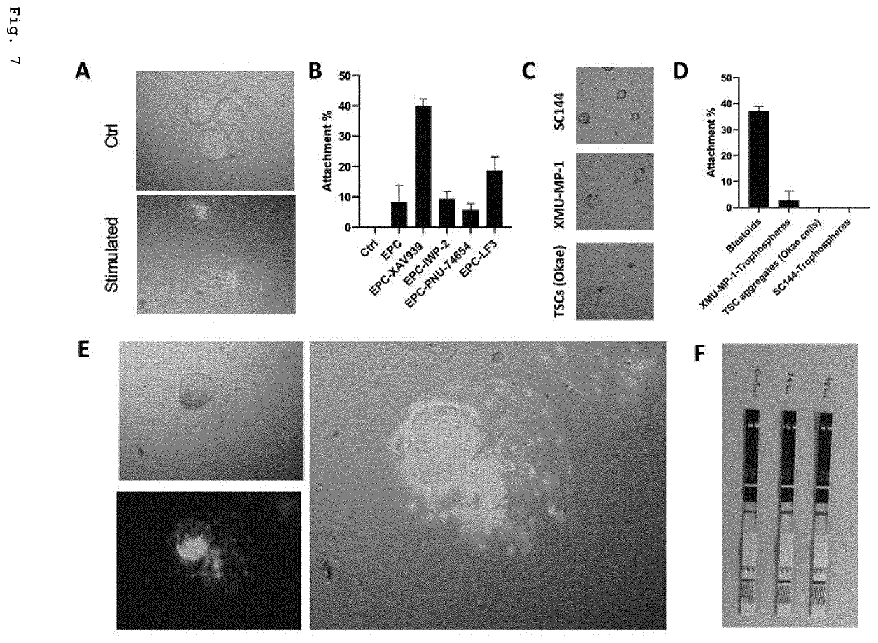

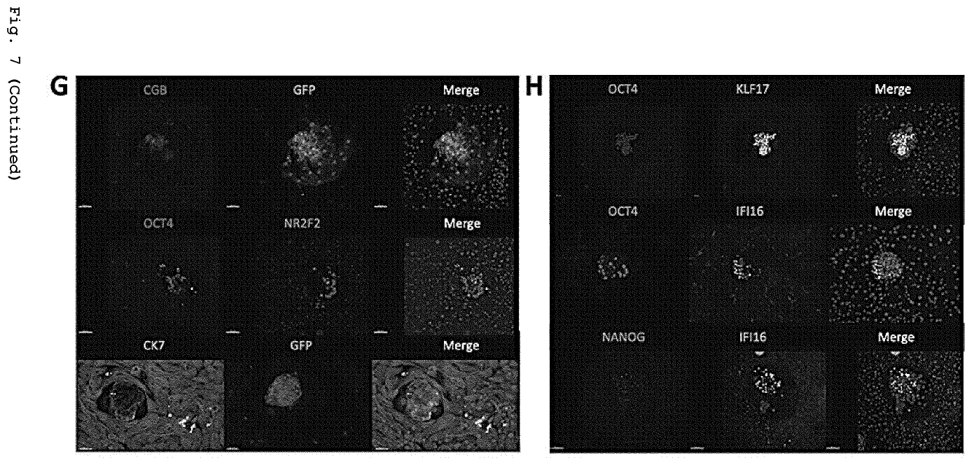

In order to model blastocyst implantation in vitro, we have defined conditions under which endometrial cells originating from organoids can be deposited in 2D culture plates and stimulated with molecules that allow them differentiate into the cells lining the uterus at the time of implantation (so called Window of Implantation, WOI). We discovered that, similar to hormones (Estradiol, Progesterone), the inhibition of the Wnt signaling pathway using multiple small molecules induces an upregulation of the expression of genes highly expressed during the WOI (FIG. 6). The inhibition of Wnt increases the expression of PAEP, SPP1, LIF and DPP4, as seen using RTqPCR of endometrial cells cultured with XAV939. The stimulated endometrial cells express PAEP also at the protein level, as shown using immunofluorescence and the cells are proliferating, as seen by the incorporation of EdU. The endometrial cells also contain subpopulations of ciliated cells that specifically express acetylated alpha tubulin and glandular cells that specifically express FOXA2, as seen using immunofluorescence.

### Example 6: Implantation of Human Blastoids into Receptive Endometrium Organoids

Upon deposition of human blastoids onto EPC/XAV939-treated endometrial cells, blastoids attach and their cells invade the endometrial layer (FIG. 7). Upon attachment and invasion, the blastoid cells maintain the expression of the pluripotency transcription factor Oct4. When endometrial cells are not stimulated with EPC/XAV939, blastoids largely fail to attach and to invade the endometrial layer. When blastoids largely fail to form an inner cluster of epiblast-like cells and hypoblast-like cells (SC144-Trophospheres, XMU-MP-1-Trophospheres), they largely fail to attach and to invade the endometrial layer. Aggregates formed from human trophoblast stem cells (Okae et al. 2018) that reflect post-implantation cytotrophoblasts fail to attach and invade the endometrial layer.

### Example 7: Extended Methods

### Culture of Human Naive Pluripotent Stem Cells

Experiments were done using the following hPSC lines; hESC lines: H9, Shef6 and HNES1. hiPSC lines: CR-NCRM2 and niPSC 16.2.b. The naïve state H9 and H9-GFP lines were provided by the laboratory of Yasuhiro Takashima. Other naive hESCs and hiPSCs were provided by the laboratory of Austin Smith. Naive hPSCs were cultured on gelatin-coated plates including a feeder layer of gamma-irradiated mouse embryonic fibroblasts (MEFs) in PXGL medium. PXGL medium is prepared using N2B27 basal medium supplemented with PD0325901 (1 μM, MedChemExpress, HY-10254), XAV-939 (1 μM, MedChemExpress, HY-15147), Go 6983 (2 μM, MedChemExpress, HY-13689) and human leukaemia inhibitory factor (hLIF, 10 ng/ml, in-house made). N2B27 basal medium contained DMEM/F12 (50%, in house made), Neurobasal medium (50%, in-house made), N2 supplement (Thermo Fisher Science, 17502048), B-27 supplement (Thermo Fisher Science, 17504044), GultaMAX supplement (Thermo Fisher Science, 35050-038), Non-essential amino acid, 2-Mercaptoethanol (100 μM, Thermo Fisher Science, 31350010), and Bovine Serum Albumin solution (0.45%, Sigma-Aldrich, A7979-50ML). Cells were routinely passaged as single cells every three to four days.

### Culture of Primed Pluripotent ESCs

Primed H9 cells were cultured on Vitronectin XF (STEMCELL Technologies, 07180) coated plates (1.0 ug/cm2) using Essential 8 medium.

### Microwell Arrays

Microwell arrays comprising microwells of 200 μm diameter were imprinted into 96-well plates.

### Induction of Blastoids and Trophospheres

Naive hPSCs or primed hPSCs cultures were treated with Accutase (Biozym, B423201) at 37° C. for 5 min, followed by gentle mechanical dissociation with a pipette. After centrifugation, the cell pellet was resuspended in PXGL medium, supplemented with Y-27632 (10 μM, MedChemExpress, HY-10583). To exclude MEF, the cell suspension was transferred onto gelatin coated plates and incubated at 37° C. for 70 min. After MEF exclusion, the cell number was determined using a Countess™ automated cell counter (Thermo Fisher Scientific) and Trypan Blue staining to assess cell viability. The cells were then resuspended in N2B27 media containing 10 μM Y-27632 (aggregation medium) and 3.0×10⁴ cells were seeded onto a microwell array included into a well of a 96-well plate. The cells were allowed to form aggregates inside the microwell for a period ranging from 0 to 24 hours depending on the cell lines and based on their propensity for aggregation. Subsequently, the aggregation medium was replaced with PALLY medium-N2B27 supplemented with PD0325901 (1 μM), A 83-01 (1 μM, MedChemExpress, HY-10432), hLIF (10 ng/ml), 1-Oleoyl lysophosphatidic acid sodium salt (LPA) (500 nM, Tocris, 3854) and Y27632 (10 μM). The PALLY medium was refreshed every 24 hours. After 48 hours, the PALLY medium was replaced with N2B27 medium containing 500 nM LPA and 10 μM Y-27632. At 96 hours, a blastoid is defined based on morphological similarity to B6 staged human blastocyst, as a structure composed of a monolayered cyst with an overall diameter of 150-250 μm comprising one inner cell cluster. We also verified that, beyond the morphology, blastoids form analogs of the three blastocyst cell lineages in the sequential and timely manner of blastocyst development. Blastoids reproducibly formed at high efficiency and we did not observe differences based on the number of passages after resetting in PXGL culture conditions. The effect of LPA, NAEPA (Sigma-Aldrich, N0912) and Verteporfin (Selleck Chemicals Llc, S1786) on the yield of blastoid formation was assessed by culturing naive hPSC aggregates in PALY medium complemented with molecules added every day from 0 to 96 hours. The Verteporfin treatment was executed without exposure to the light. The effect of the aPKC inhibitor CRT0103390 (Gift from the laboratory of Kathy Niakan) was assessed by culturing naive hPSC aggregates in PALLY medium complemented with 2 μM CRT0103390 every day from 0 to 96 hours. The formation of trophospheres was induced by culturing naive hPSC aggregates in PALLY medium complemented with 2 μM XMU-MP-1 (Med Chem Express, HY-100526) or 3 μM SC-144 (Axon, 2324) every day from 0 to 96 hours. The BSA concentration was titrated within the range of 0-0.3% for individual cell lines used for the formation of the blastoids and trophospheres.

Derivation of Cell Lines from Human Blastoids

Derivation experiments were performed with blastoids cultured for 96 hours as described in the previous section. Blastoids were individually transferred on gelatin coated 96-well plates with feeder layers of gamma-irradiated MEFs. Naive hPSCs were derived in PXGL medium. hTSCs were derived in human trophoblast stem cell (hTSC) medium (Okae, H. et al. Cell Stem Cell 22, 50-63.e6 (2018)). After 24 hours of culture on feeders, blastoids attached and, within one week, colonies were formed. Derivation was considered successful after three passages after blastoid transfer. For immunofluorescence assays, naive hPSCs were transferred onto Geltrex (0.5 μL/cm²) coated coverslips, and hTSCs were transferred onto Fibronectin coated coverslips (5 ug/ml, Sigma Aldrich, 08012).

### Trophoblast Organoid Formation

Organoid formation was performed with blastoid derived hTSC lines. Organoids were cultured as previously described (Turco, M. Y. et al. Nature 564, 263-267 (2018)) with some modifications. Colonies of hTSCs were dissociated into single cells using 1×Trypsin at 37° C. for 5 min. After centrifugation, 200.000 cells were resuspended in 150 ul Matrigel (Corning, 356231). Droplets of 20 ul per well were placed into a prewarmed 48-well cell culture plate and placed upside down into the incubator for 20 min. Organoids were cultured in 250 ul TOM medium (Advanced DMEM-F12, N2 supplement, B27 supplement minus vitamin a, Pen-Strep, N-Acetyl-L-Cysteine (1.25 mM), L-glutamine (2 mM), A83-01 (500 nM), CHIR99021 (1.5 μM), recombinant human EGF (50 ng/ml), 10% R-Spondin 1 conditioned medium, recombinant human FGF2 (100 ng/ml), recombinant human HGF (50 ng/ml), PGE2 (2.5. uM). Medium was refreshed every other day. For SCT formation organoids were maintained in TOM medium until day 7.

### 2D Trophoblast Differentiations

The differentiation of blastoid derived hTSCs was performed as described previously (Okae, H. et al. Cell Stem Cell 22, 50-63.e6 (2018)) with some modifications. hTSC lines were adapted to Fibronectin coating (5 ug/ml, Sigma Aldrich, 08012) for at least three passages prior to the experiments. For EVT and SCT differentiation, cells were dissociated with TrypLE for 5 min at 37° C. and cells were seeded at a density of 55.000 cells/well onto 12-well plates. For SCT differentiation, the plates were precoated with 10 ug/ml Fibronectin and cultured in SCT medium (DMEM/F12, supplemented with 0.1 mM 2-mercaptoethanol, 0.5% Penicillin-Streptomycin, 1% ITS-X supplement, 7.5 mM A83-01, 2.5 mM Y27632, 4% KnockOut Serum Replacement and 2 mM forskolin) for 3 days. For EVT differentiation, plates were precoated with Matrigel and cells were cultured in EVT medium (DMEM/F12, supplemented with 0.1 mM 2-mercaptoethanol, 0.5% Penicillin-Streptomycin, 1% ITS-X supplement, 2% Matrigel, 7.5 mM A83-01, 2.5 mM Y27632, 4% KnockOut Serum Replacement and 100 ng/ml NRG1,). After three days, the medium was changed to EVT medium with 0.5% Matrigel and without NRG1. Cells were cultured until day 6.

### Human Pre-Implantation Embryos Culture

Human embryos were thawed following the manufacturer's instructions (Cook Medical: Sydney IVF Thawing kit for slow freezing and Vitrolife: RapidWarmCleave or RapidWarmBlast for vitrification). Human embryos frozen at 8-cell stage were loaded in a 12-well dish (Vitrolife: Embryoslide Ibidi) with non-sequential culture media (Vitrolife G2 plus) under mineral oil (Origio: Liquid Paraffin), at 37° C., in 5% O₂/6% CO₂.

### Plasmid Construction

The cDNA sequence of hYAP1, hYAP1 5SA, and hYAP1 5SA+S94A were amplified from the pQCXIH-Myc-YAP, pQCXIH-Myc-YAP-5SA, pQCXIH-Myc-YAP-S94A plasmids respectively. These YAP plasmids were gifts from Kunliang Guan (Addgene plasmid #33091, #33093 and #33094) (Zhao, B. et al. Genes Dev. 21, 2747-2761 (2007)). The individual cDNA sequences were cloned into pDONR211, followed by cloning into PB-TAC-ERP2 using Gateway (invitrogen) cloning strategy. PB-TAC-ERP2 was a gift from Knut Woltjen (Addgene plasmid #80478) (Kim, S.-I. et al. Methods Mol. Biol. 1357, 111-131 (2016)).

### Cell Transfection in Human Naive PSCs

pCAG-PBase (5 μg) and PB-TAC-YAP1-ERP (5 μg) were transfected by NEPA21 electroporation (Nepa Gene Co. Ltd) into 5×10⁴ cells in single-cell suspension. Electroporated naive hPSCs were plated on Geltrex (0.5 μL/cm², Thermo Fisher Science, A1413302)-coated 6-well plates with PXGL medium containing Y-27632 (10 μM). Puromycin (0.5 ug/ml, Sigma-aldrich, P7255) was added to PXGL medium from day 1 to day 3-4 to select transformed cells. pCAG-PBase was a gift from Knut Woltjen.

YAP Overexpression in Naive hPSC Aggregates

The naive hPSC aggregates were formed from naive H9 cell lines integrated with the doxycycline inducible cassette as described in the section above. The aggregates were cultured in PALLY medium with reduced LPA concentration (5 nM) from 0 hours to 48 hours along with 100 ng/ml Doxycycline. Higher LPA concentrations masked the effects of the genetic overexpression of the YAP1 variants. The number of cavitated aggregates were counted at 72 hours.

### Single Cell RNA-Seq Library Preparation and Sequencing

To avoid over-representation of TE cells, blastoids were collected, dissociated and the cell suspension was stained using antibodies against TROP2 and PDGFRa that mark trophoblasts and primitive endoderm, respectively. For the 96 hours time point, blastoids were selectively picked up from the microwell arrays before the dissociation, according to the morphological criterion described above. Cells were FACS-sorted into 384 well-plates containing the lysis buffer for Smart-seq2 and immediately frozen. The antibody staining was exploited in order to harvest specific numbers of TROP2+, PDGFRa+ and double-negative cells. The abuted FACS gates (DiVa 9.0.1) covered the whole spectrum and no blastoid cells were excluded. The H9 naive cells cultured on MEF were stained using an antibody against SUSD2, then FACS-sorted. Dead cells were excluded by DAPI staining. Smart-seq2 libraries were generated as described previously with minor optimization (Picelli, S. et al. Nat. Protoc. 9, 171-181 (2014)). Maxima H Minus reverse transcriptase (3 U/reaction, Thermo Fisher Science, EP0751) was used for the CDNA synthesis. The prepared libraries were sequenced on the S1 or SP flow cell using an Illumina Novaseq instrument in 50 bp paired end mode.

### Single Cell RNA-Seq Data Analysis

Smart-Seq transcriptome sequencing experiments were analysed using genome sequence and gene annotation from Ensembl GRCh38 release 103 as reference.

For gene expression quantification RNA-seq reads were first trimmed using trim-galore v0.6.6 and thereafter aligned to the human genome (Ensembl GRCh38 release 103) using hisat2 v2.2.1. Uniquely mapping reads in genes were quantified using htseq-count v0.13.5 with parameter-s no. TPM estimates were obtained using RSEM v1.3.3 with parameter -single-cell-prior. Further analysis was performed in R v4.0.3 with Seurat v4.0.1. Based on initial evaluation of per-cell quality control metrics and outlier identification using the median absolute deviation algorithm, cells with <=2000 detected genes or >=12.5% mitochondrial gene percentage were filtered out. Only genes detected in at least 5 cells were retained. Count-data were log-normalized, top 3000 highly variable were selected, and standardization of per gene expression values across cells was performed using NormalizeData, FindVariableFeatures and ScaleData data functions in Seurat. Principal component analysis (PCA) based on the standardized highly variable features was used for linear dimension reduction, a shared nearest neighbor (SNN) graph was constructed on the dimensionally reduced data, and the graph was partitioned using a SNN modularity optimization based clustering algorithm at a range of resolutions using RunPCA, FindNeighbors and FindClusters from Seurat with default settings. Cluster marker genes were identified with the Wilcox likelihood-ratio test using the FindAllMarkers function. Uniform Manifold Approximation and Projection (UMAP) was used for visualization. For integration of Smart-Seq experiments from multiple sources we followed the previously described procedure (Zhao, C. et al. doi: 10.1101/2021.05.07.442980). Published data from E-MTAB-3929 (human preimplantation embryos ranging from embryonic day 3 to 7 Petropoulos, S. et al. Cell vol. 167 285 (2016)), GSE109555 (in vitro cultured blastocysts) were downloaded, and data from Carnegie stage 7 embryo was kindly provided by the authors (Tyser, R. C. V. et al. bioRxiv (2020) doi: 10.1101/2020.07.21.213512). All the data was preprocessed to obtain per gene read counts using the same protocol as described for blastoid cells, in the case of GSE109555 including adaptations to accommodate UMI and CB information following the authors instructions (https://github.com/WRui/Post_Implantation). For GSE109555 we used 1000 cells randomly subsampled from the 3184 high quality single cells described in the original publication. We excluded cells belonging to hemogenic endothelial progenitors and erythroblasts. After evaluation of per-cell quality control metrics, cells with >2000 detected genes and <12.5% mitochondrial gene percentage were retained. Genes detected in at least five cells in any dataset were retained. Log-normalization was performed using computeSumFactors in scran package v1.18.7, per-batch scaling normalization using multiBatchNorm in batchelor v1.6.3. Datasets were aligned using the fastMNN approach via SeuratWrappers v0.3.0 using the log-normalized batch-adjusted expression values. MNN low-dimensional coordinates were then used for clustering and visualization by Uniform Manifold Approximation and Projection (UMAP).

### Culture of Human Trophoblast Stem Cells and Aggregate Formation

Experiments were performed using the human blastocyst-derived hTSC line bTS5 provided by the laboratory of Takahiro Arima. Cells were cultured on Laminin 511 (5 μg/ml, BioLamina, LN511) coated plates in hTSC medium as previously described¹². Aggregates of hTSCs were formed as follows. Colonies were dissociated into single cells using Accutase at 37° C. for 5 min. The cells were resuspended into hTSC medium containing 10 μM Y27632, and 3.0×10⁴ cells were seeded onto a microwell array imprinted into a well of a 96-well plate. The same medium (Okae, H. et al. Cell Stem Cell 22, 50-63.e6 (2018)) was refreshed daily. After 72 hours, the aggregates were used for both characterisation and implantation experiments.

### Endometrial Organoid Culture

Cryopreserved human endometrial organoids were provided by the Hossein Baharvand laboratory (Royan Institute) within the framework of collaboration agreements. Human endometrial organoids were established from healthy human donors following the protocol described previously (Boretto, M. et al. Development 144, 1775-1786 (2017)) with some modifications. Briefly, organoids were cultured in human endometrial expansion medium composed of 10% Rspol conditioned medium (in-house made) and 10% Noggin-Fc-conditioned medium (Heijmans, J. et al. Cell Rep. 3, 1128-1139 (2013)), supplemented with 1× N2 supplement, 1× B27 supplement, 1× Insulin-Transferrin-Selenium (in-house), Glutamax (1 μM), N-acetylcysteine (1.25 mM, Sigma-Aldrich, A7250), nicotinamide (2.5 mM, Sigma-Aldrich, 72340), EGF (50 ng/ml, Peprotech, 100-47), bFGF (2 ng/ml, Peprotech, 100-18B), HGF (10 ng/ml, Peprotech, 315-23), FGF10 (10 ng/ml, Peprotech, 100-26), A83-01 (500 nM) and SB202190 (10 μM, Tocris, 1264). Y-27632 (10 UM) was used in the first 2 days after passaging to prevent apoptosis. The medium was changed every 2 days and the organoids were passaged with TrypLE followed by mechanical dissociation every 7-9 days.

### Hormonal Stimulation of Endometrial Organoids and OFELs Culture

Endometrial organoids were passaged as described in the previous section. The dissociated cells were resuspended in Matrigel supplemented with Y-27632 (10 μM), cell suspension was deposited in 48-well plates and were cultured in endometrial expansion medium for 2 days. The organoids were stimulated first with E2 (10 nM, Sigma-Aldrich, E2758) for 2 days, followed by the mixture of E2 (10 nM), P4 (1 μM, Sigma-Aldrich, P8783), and CAMP (250 μM, Biolog, B 007) with or without XAV939 (10 UM) (EPC or EPCX respectively) for 4 days. For OFELs culture, organoids were recovered from the matrigel droplets with ice-cold DMEM/F12 and mechanical pipetting. The organoids were dissociated using TrypLE and mechanically triturated to generate single cells and seeded at the density of 3 to 3.5×10⁴ cells per well into a 96-well glass bottom plate (Cellvis, P96-1.5H-N) and cultured for 2-3 days with stimulation. For contraceptive treatment, levonorgestrel (LNG) (10 μM, Sigma-Aldrich, PHR1850) was added every day to the medium two days after hormonal stimulation and continued until the end of experiment.

### In Vitro Implantation Assay

Confluent OFELs were prepared for the implantation assay at least 2 hours prior to the deposition of blastoids, trophospheres, naive hPSCs or hTSCs aggregates by washing the OFEL two times with DMEM/F12 and adding IVC medium (Xiang, L. et al. Nature 577, 537-542 (2020)). Structures were then transferred onto the OFELs using a mouth pipette under an inverted microscope. After 24-48 hours, the medium was removed, the well was washed with PBS, fixed using 4% formaldehyde for 30 minutes at room temperature and subsequently processed for immunofluorescence staining. The percentage of attached structures was reported as the percentage of total transferred structures.

### In Vitro Culture of Human Blastoids in Post Implantation Conditions

Human blastoids were selected using a mouth pipette, washed with CMRL1066 medium and transferred into suspension culture plates or 96-well plates coated with Matrigel containing pre-equilibrated media adapted from monkey blastocyst culture (Ma, H. et al. Science 366, (2019).) with minor modifications as followed. For the first day, the culture medium was CMRL1066 supplemented with 10% (v/v) FBS, 1 mM l-glutamine (Gibco), 1×N2 supplement, 1× B27 supplement, 1 mM sodium pyruvate (Sigma) and 10 μM Y27632. After 24 h, half of the medium was replaced with a new medium including 5% Matrigel. After 48 h, 50% of medium was replaced with a new medium supplemented with 20% (v/v) FBS and 5% Matrigel. After 72 h, half of the medium was replaced with a new medium supplemented with 30% (v/v) KSR and 5% Matrigel. Then, half of the medium was replaced every day and blastoids were cultured for up to 6 days. Cultures were fixed for staining after 4 and 6 days of in vitro culture with 4% PFA as mentioned above.

### Human Pre-Implantation Embryos in Comparative Example

The use of human embryos donated to research as surplus of IVF treatment was allowed by the French embryo research oversight committee: Agence de la Biomédecine, under approval number RE13-010 and RE18-010. All human pre-implantation embryos used in this study were obtained from and cultured at the Assisted Reproductive Technology unit of the University Hospital of Nantes, France, which are authorized to collect embryos for research under approval number AG110126AMP of the Agence de la Biomédecine. Embryos used were initially created in the context of an assisted reproductive cycle with a clear reproductive aim and then voluntarily donated for research once the patients have fulfilled their reproductive needs or tested positive for the presence of monogenic diseases.

RNA Extraction, cDNA Synthesis and qRT-PCR

RNA was extracted using the RNeasy mini kit (Qiagen, 74106) and cDNA synthesis was performed using the Superscript III (Invitrogen, 18080093) enzyme. qPCR reactions were performed using GoTaq® qPCR Master Mix (Promega, A6001) on CFX384 Touch Real-Time PCR Detection System (Bio-rad). Quantification was performed using Microsoft Office Excel by applying the comparative Cycle threshold (Ct) method. Relative expression levels were normalized to GAPDH.

### ELISA Assay for CGβ Detection

Media from wells containing unattached or attached blastoids was collected and centrifuged to remove debris and stored at −80° C. until use. The supernatant was subject to CGβ ELISA (Abcam, ab178633), according to the manufacturer's instructions, alongside CGβ standards.

### Ligand-Receptor Analysis

The Cellinker web-platform was used to predict putative receptor-ligand interactions between polar TE and endometrial epithelial cells. Genes within the NR2F2 module for late TE, enriched genes in polar TE, gene module for endometrial epithelial cells that marked the entrance into phase 4 of menstrual cycle along with upregulated genes in stimulated OFELs were used as the query to search ligands and receptors in the database.

### Immunohistochemistry

The samples were fixed with 4% formaldehyde for 30 minutes at room temperature. Post fixation, formaldehyde solution was removed and the samples were washed at least three times with PBS. The samples were then permeabilized and blocked using 0.3% triton-x 100 and 10% normal donkey serum in PBS for at least 60 minutes. The samples were then incubated overnight at 4° C. with primary antibodies diluted in fresh blocking/permeabilization solution. The samples were washed with PBS containing 0.1% triton-x100 (PBST) at least three times for 10 minutes each. The washing buffer was then replaced with Alexafluor tagged secondary antibodies (Abcam or Thermofisher scientific) along with a nuclear dye Hoechst-33342 (1:500 or 1:300 for 2D or 3D samples respectively, Life Technologies, H3570) diluted in PBST for 30 minutes in dark at room temperature. The samples were then washed with PBST three times for 10 minutes each. For human blastocysts, the samples were fixed at the B4 or B6 stage according to the grading system proposed by Gardner and Schoolcraft or at B3 or B4+72 hours in vitro culture. Embryos were fixed with 4% paraformaldehyde for 10 minutes at room temperature and washed in PBS/BSA. Embryos were permeabilized and blocked in PBS containing 0.2% Triton-x100 and 10% FBS at room temperature for 60 min. Samples were incubated with primary antibodies overnight at 4° C. Incubation with secondary antibodies was performed for 2 hours at room temperature along with Hoechst counterstaining. The samples were mounted for imaging in PBS in the wells of glass bottom micro slides (Ibidi, 81507). EdU staining was done using Click-iT EdU Alexa Fluor 647 Imaging Kit (Thermo Scientific, C10640) following the manufacturer's instructions.

### Microscopy and Image Analysis

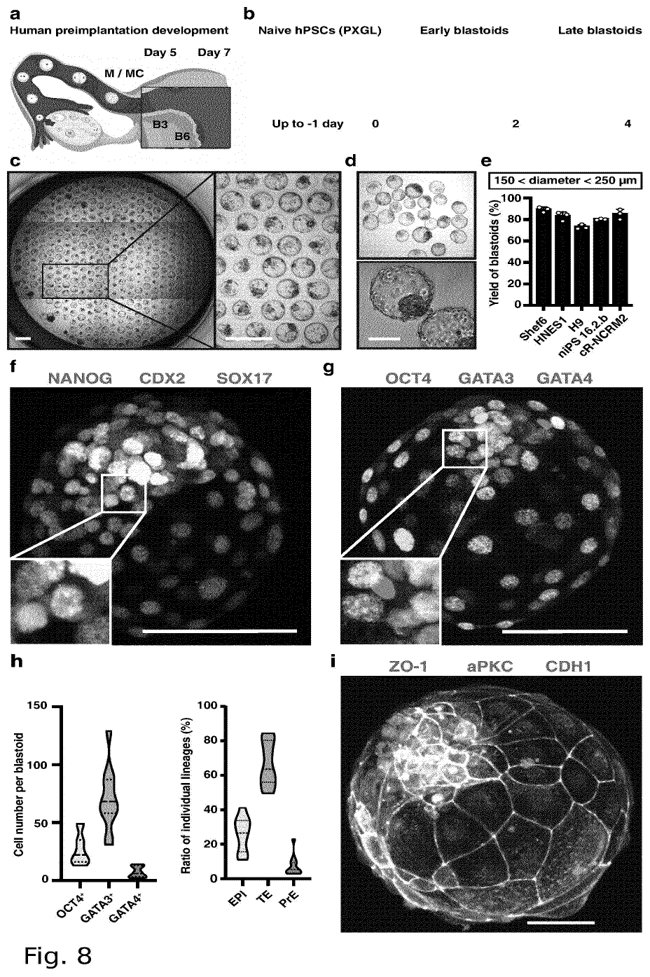

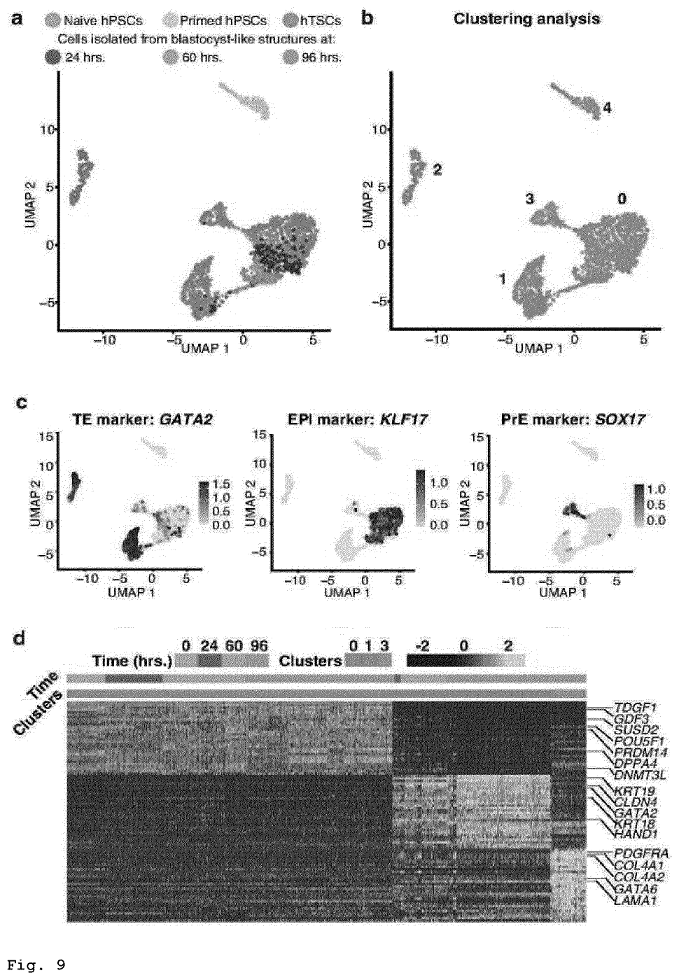

The phase contrast images were acquired using Thermo Fisher scientific EVOS cell imaging system and inverted wide field microscope Axio VertA1. The number of blastoids or cavitated structures were counted manually for each well. After 96 hours, a blastoid is defined based on the morphological parameters as described in previous sections. The fluorescent images and timelapse images were acquired using Olympus IX83 microscope with Yokogawa W1 spinning disk (Software: CellSense 2.3; camera: Hamamatsu Orca Flash 4.0) or Nikon Eclipse Ti E inverted microscope, equipped with a Yokogawa W1 spinning disc (Software: Visiview 4.5.0.7; camera: Andor Ixon Ultra 888 EMCCD). The confocal images were analyzed and display images were exported using FIJI 1.53k or Bitplane Imaris 9.7.0 softwares. For cell counting, Bitplane Imaris software was used. Cell count parameters were set for size and fluorescence strength of voxels and then overall cell count data was obtained for each image using Imaris's spot function. Note that large cavities in blastoids increase the depth of the imaging field causing poor signal from deeply located cells. Therefore, our counting data in FIG. 8H could be underrepresented values, particularly in the case of trophectoderm cells. The quantification of the percentage of blastoids forming the NR2F2 axis was done manually. To do so, blastoids stained to detect NR2F2 expression were imaged using a confocal-spinning disk microscope. The images were projected using a 3D-project function in FIJI. The blastoid was classified to have an axis when NR2F2 expression was restricted to its polar half with no expression or lower level of expression in the mural half. The inverted pattern of NR2F2 expression was classified as an invert axis. The blastoids with NR2F2 expression on their both polar and mural halves were classified to have no axis. Confocal immunofluorescence images of human blastocysts were acquired with a Nikon confocal microscope and a 20× Mim or 25× Silicon objective. Optical sections of 1 μm-thick were collected. The images were processed using Fiji (http://fiji.sc) and Volocity 6.3 visualization softwares. Volocity software was used to detect and count nuclei.

### Statistics and Reproducibility

All the experiments were performed at least in three biological replicates unless specifically described in the methods and the figure legends. Statistical analyses were performed using Graphpad prism 8.1.1 (330).

### Example 8: Triple Inhibition (Hippo/ERK/TGF)

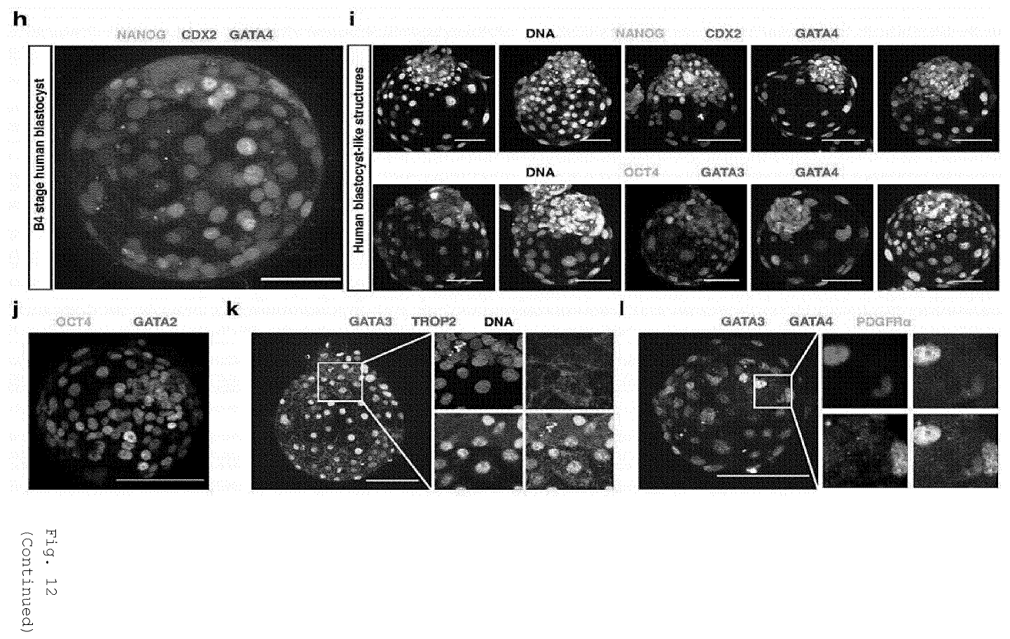

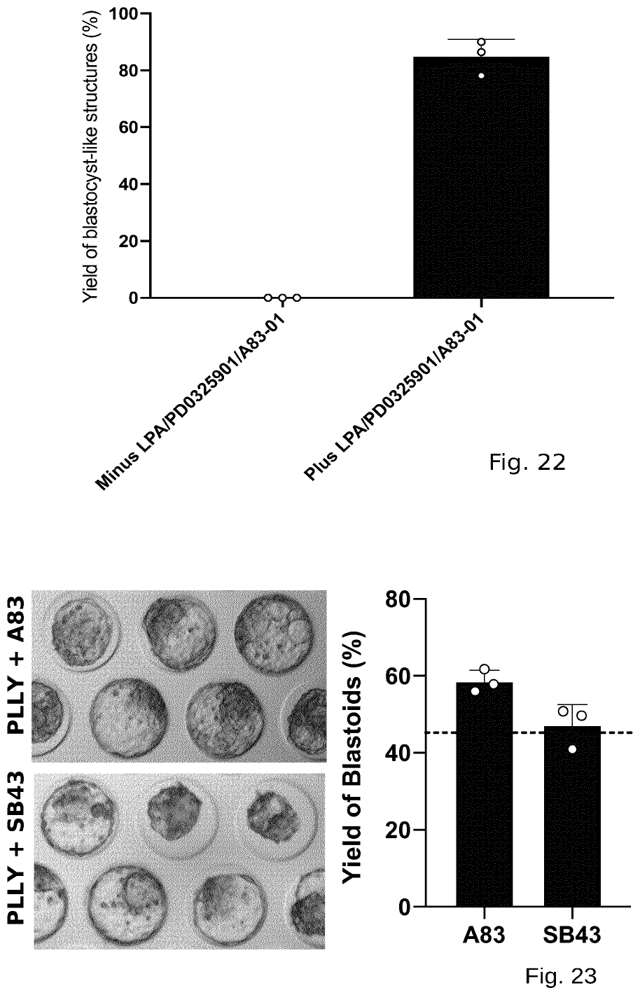

The blastocyst forms within 3-4 days by generating the conceptus 3 founding lineages: the epiblast (EPI, embryonic), trophectoderm (TE, extraembryonic) and primitive endoderm (PrE, extraembryonic) (FIG. 8a). Peripheral cells become TE by inhibiting the Hippo pathway. Also, naive hPSCs (cultured in PXGL) efficiently form TE analogs upon inhibition of TGF-β and ERK pathways. Therefore, we aggregated naive hPSCs in non-adherent hydrogel microwells and inhibited these three pathways (FIG. 8b & FIG. 12a-c). Upon exposure to Lysophosphatidic Acid (LPA, Hippo pathway inhibitor), A83-01 (TGF-β family receptors inhibitor) and PD0325901 (ERK inhibitor) in a chemically-defined medium containing LIF (STAT activator) and Y-27632 (ROCK inhibitor), blastocyst-like structures formed efficiently (FIG. 8c-e, >70%, 150<ø<250 μm, see full morphometric criterion in extended methods), and consistently (FIG. 12d, >20 passages). LPA significantly improved efficiency (FIG. 12b-d). Within 5 days the cell number (47+/−9 to 129+/−27) and overall size (65 to 200 μm) increased (FIG. 12e, f) as in the range of day 5-7 blastocysts (stages B3-6). TE cells analogs (GATA2⁺/GATA3⁺/CDX2⁺/TROP2⁺) formed, proliferated (FIG. 8f-h & FIG. 12g-1), established adherens junctions (Epithelial cadherin (CDH1)), apical-basal polarity (aPKC localization) and tight junctions (ZO-1⁺, FIG. 8i and FIG. 12m) while undergoing cycles of inflations/deflations (FIG. 12n). Strikingly, all blastocyst-like structures set apart a unique inner cell cluster reflecting the EPI (OCT4⁺; average=27+/−13 cells; 26% of total cells) and PrE (GATA4⁺/SOX17⁺/PDGFRa⁺ average=7+/−5 cells; 7% of total cells) (FIG. 8f-h & FIG. 12i, j, l) which indicates breaking of symmetry after cell aggregate formation. Multiple lines of naive hESCs (Shef6, H9, HNES1) and hiPSCs (niPSC 16.2.b, cR-NCRM2) formed such structures with comparable high efficiency (FIG. 8e & FIG. 12o), while primed hPSCs that reflect the post-implantation EPI did not (FIG. 12p). The tripple inhibition of inhibiting Hippo pathway (e.g. with LPA), MEK/ERK (e.g. with PD0325901) and TGF-b (e.g. with A83-01) was necessary to develop blastoids with a cavity based on human cells (FIG. 22). FIG. 23 shows that alternative TGF-b inhibitors (SB431542) also work.

### Example 9: Formation of Blastocyst Analogs Only

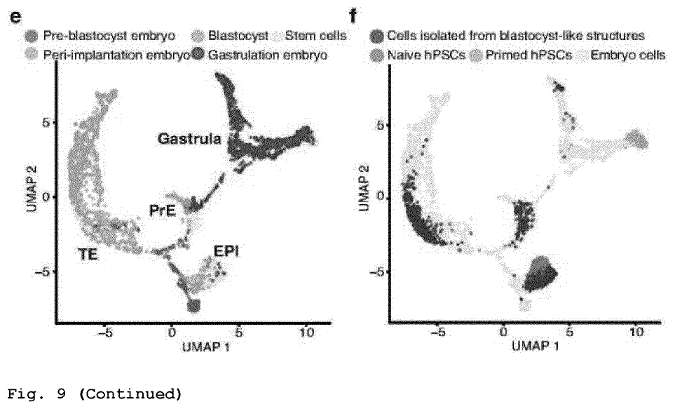

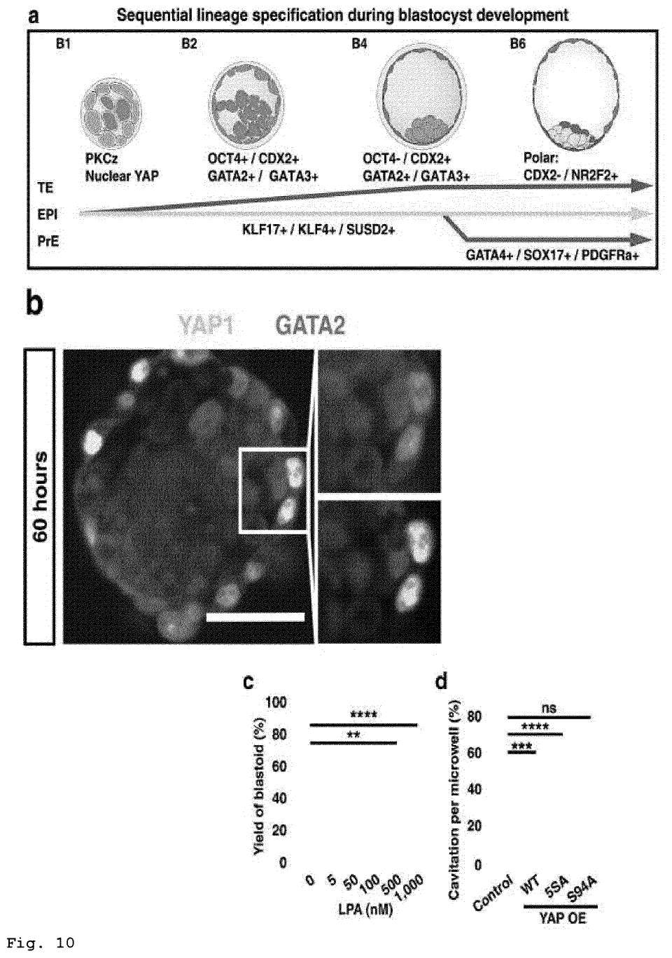

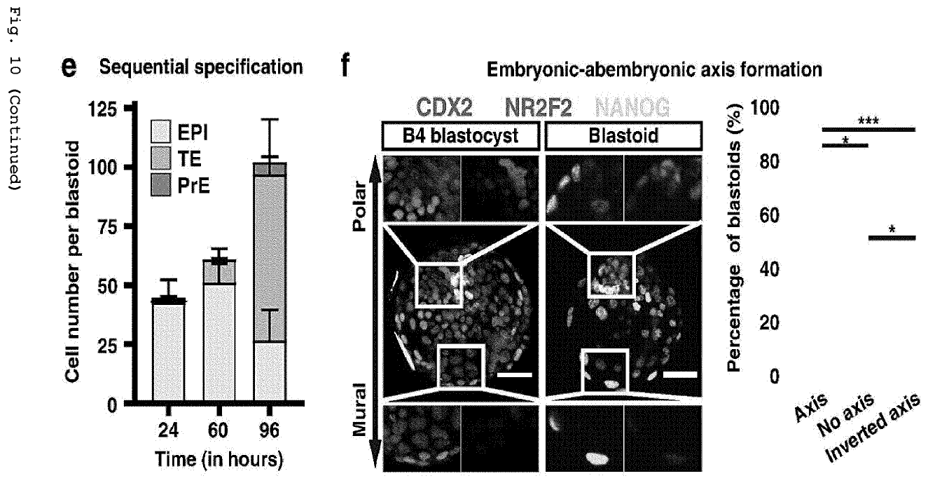

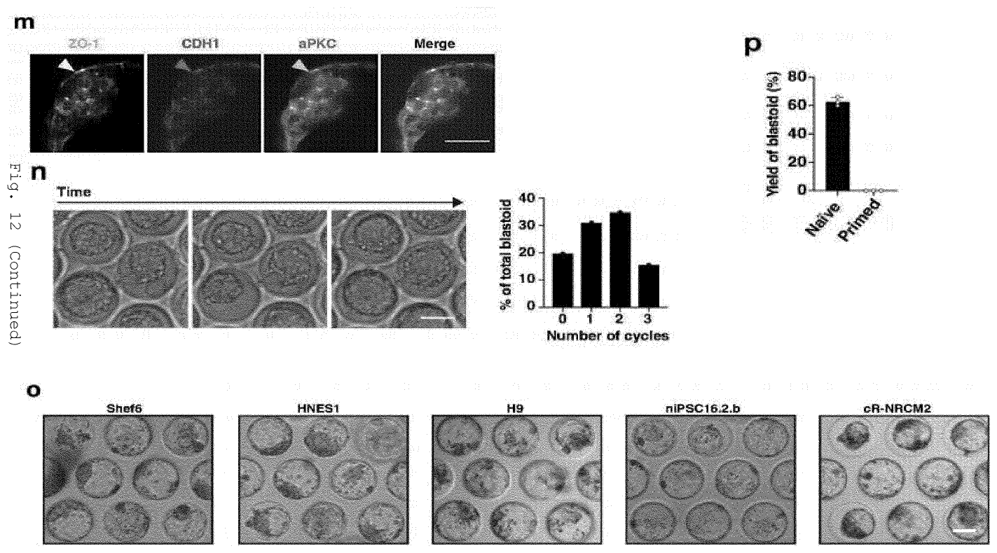

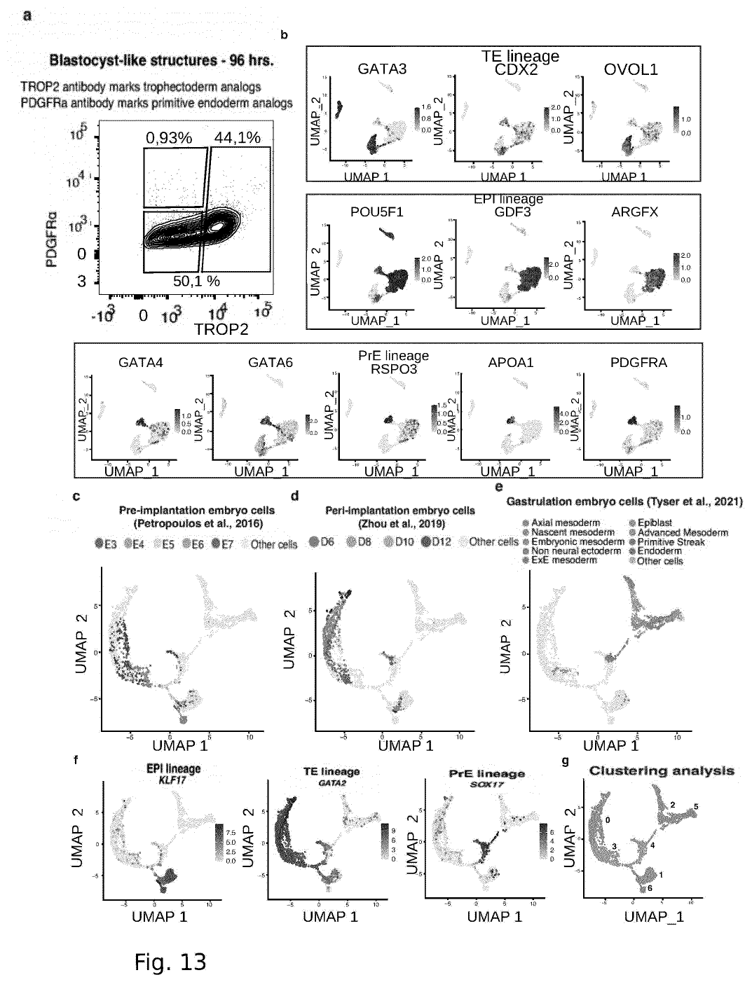

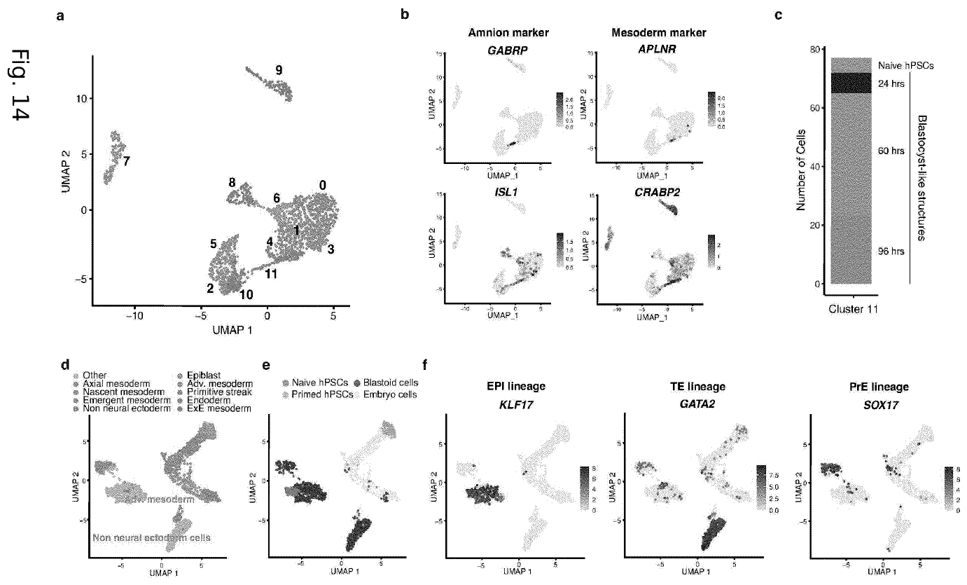

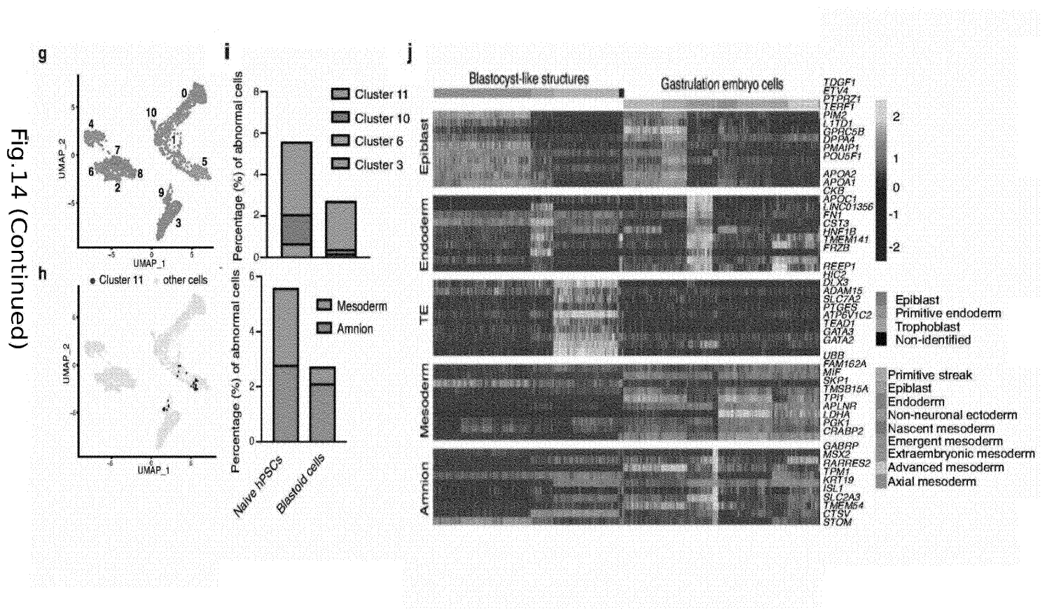

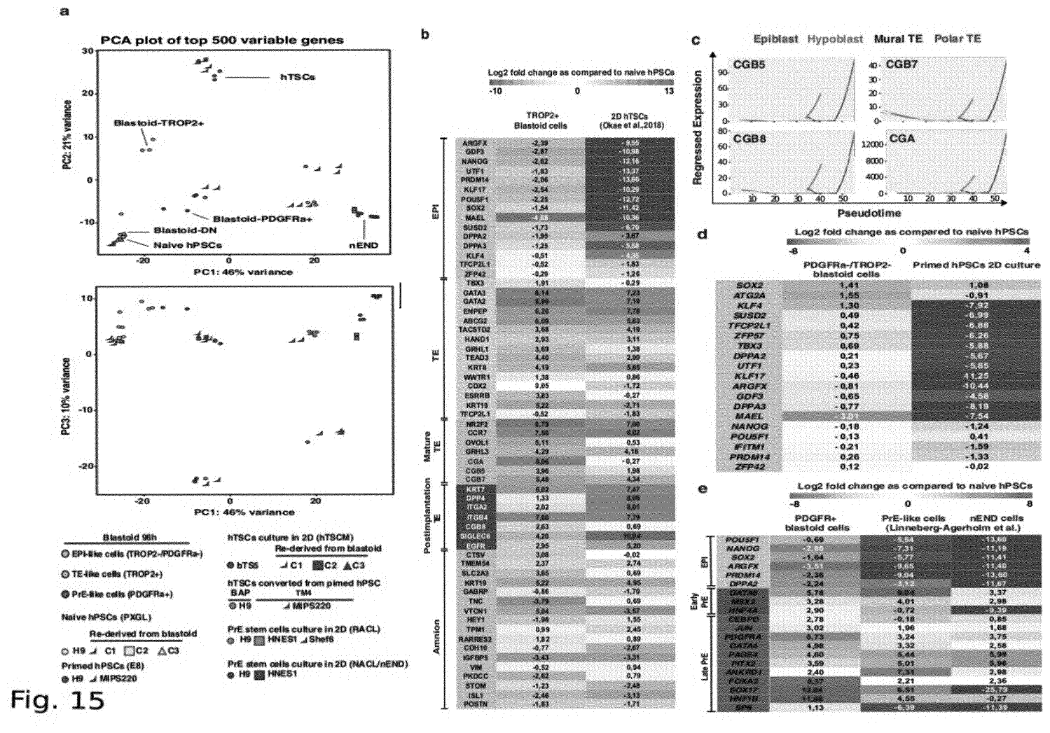

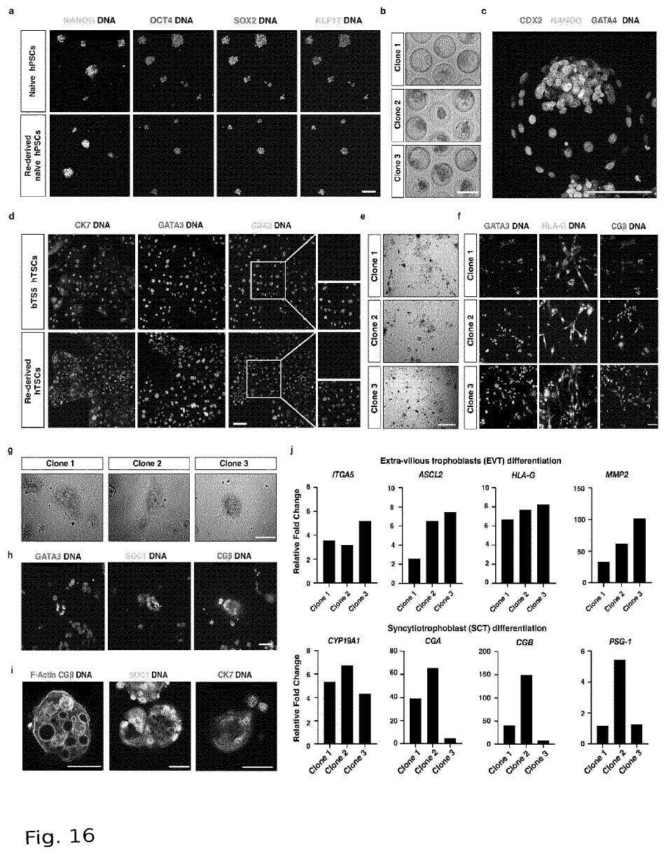

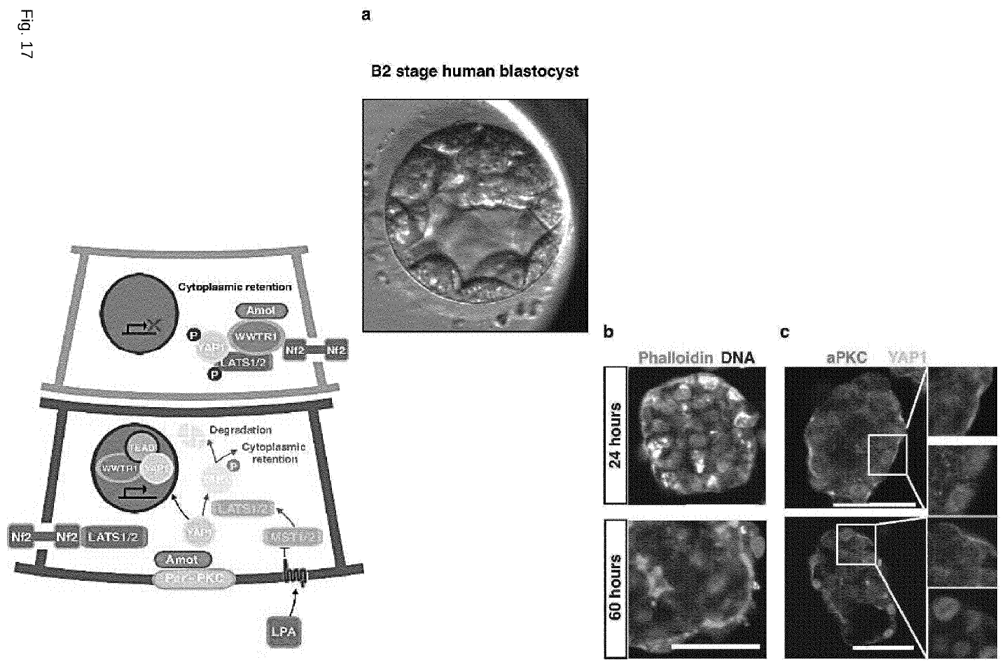

Single cell transcriptomics analysis showed that blastocyst-like structures formed three main transcriptomic states (FIG. 9a, b & FIG. 13a) marked by genes specific to the three founding lineages, including GATA2/GATA3 (TE), POU5F1/KLF17 (EPI), and GATA4/SOX17 (PrE) (FIG. 9c, d & FIG. 13b). Comparison with cells from blastocysts, in vitro cultured blastocysts, and a gastrulation-stage embryo disclosed that cells were transcriptionally similar to the blastocyst state and distinct from postimplantation states (FIG. 9e, f & FIG. 13c-g). A higher-resolution clustering analysis (×50) isolated one cluster of non-blastocyst-like cells with a gene expression pattern reminiscent of post-implantation tissues (GABRP, ISL1, APLNR, CRABP2) (FIG. 14a-c) and which appeared transcriptionally similar to amnion (annotated as non-neural-ectoderm) and mesoderm (FIG. 14d-j). This sub-population constituted less than 3% of the cells (FIG. 14i). Of note, naive hPSCs culture comprised 5,6% of similarly differentiated cells (FIG. 14i). Bulk RNA sequencing analysis showed that isolated trophoblast analogs (TROP2⁺ by flow cytometry) had an intermediate transcriptome between naive hPSCs and post-implantation-like trophoblasts (hTSCs) (FIG. 15a). Furthermore, trophoblasts were enriched in blastocyst-stage TE transcripts (ESRRB, GRHL1, OVOL1, GATA2, GATA3, TBX3, KRT19, CGA, CGB5, CGB7) but not in some post-implantation markers (SIGLEC6, DPP4) (FIG. 15b, c). The transcriptome of isolated EPI analogs (TROP2-/PDGFRa-) resembled the one of naive hPSCs (FIG. 15a) and was enriched in markers specific for blastocyst-stage EPI (KLF17, ATG2A, SUSD2, TFCP2L1, ZFP57, DPPA2, UTF1, PRDM14) and differed from the transcriptome of primed hPSCs (FIG. 15a, d). Finally, isolated PrE analogs (PDGFRa⁺) had an intermediate transcriptome between naive hPSCs and extraembryonic endoderm cell lines (nEND cells) (FIG. 15a), were enriched in blastocyst-stage PrE markers (Early blastocyst: GATA6, MSX2, HNF4A. Late blastocyst: PDGFRA, GATA4, SOX17, HNF1B, FOXA2), and downregulated EPI genes (ARGFX, PRDM14, SOX2, NANOG, DPPA2, POU5F1), as in blastocysts (FIG. 15e). Blastocysts have the ability to establish stem cell lines. Similarly, blastocyst-like structures permitted de novo derivation of naive hPSCs (NANOG⁺/SOX2⁺/OCT4⁺/KLF17⁺) (FIG. 16a) able to form 2^{nd} generation blastocyst-like structures (FIG. 16b, c) and hTSCs (CDX2⁻/GATA3⁺/CK7⁺) (FIG. 16d) endowed with rapid differentiation capacity into syncytio- (SCT) and extravillous trophoblasts (EVT) (3-6 days, FIG. 16e-j). Of note, derivation of PrE cell lines from human blastocysts has not been reported previously. Altogether, because this model morphologically resembled blastocysts (see criterion in extended methods) and generated analogs of the three lineages transcriptionally reflecting the blastocyst stage and developing in the sequential and timely way of blastocysts, we referred to them as blastoids.

### Example 10: Hippo Inhibition

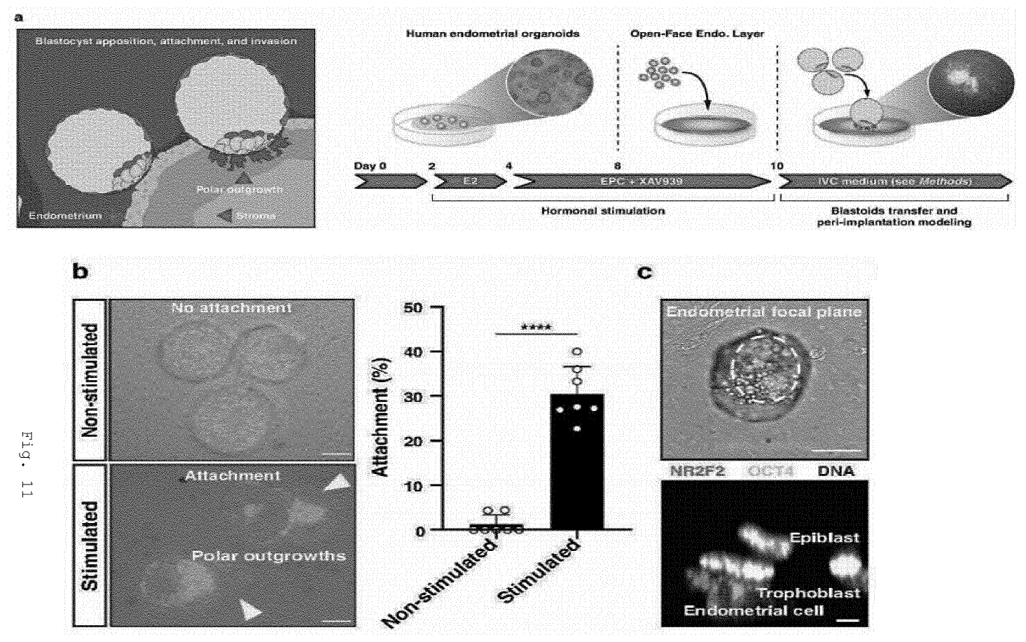

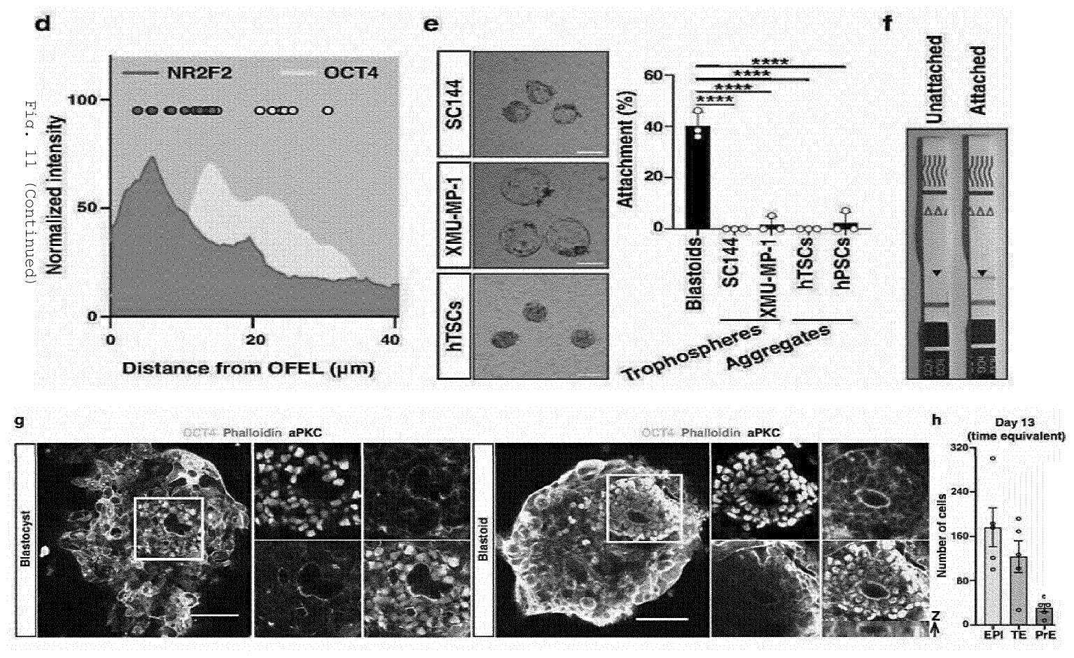

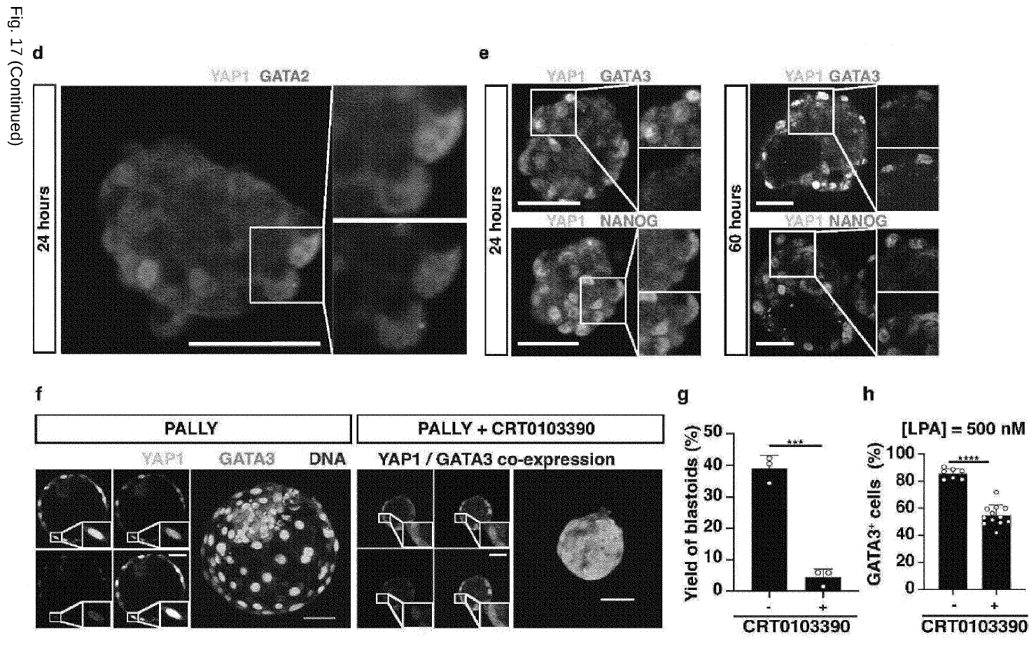

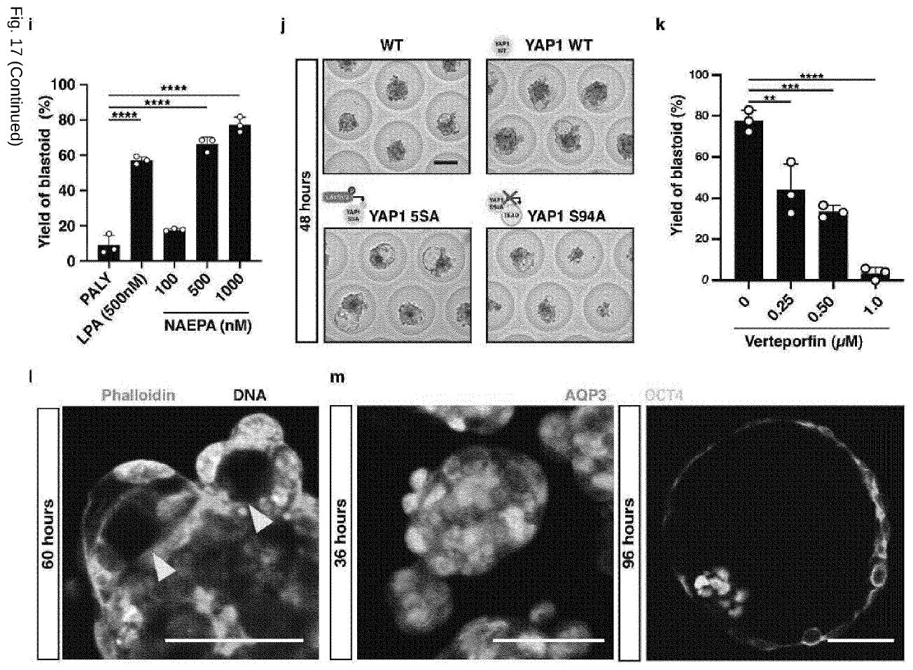

Knowledge about human blastocyst lineage segregation is limited (FIG. 10a). However, inhibition of the Hippo pathway occurs in peripheral cells upon acquisition of an apical domain and is required for trophoblast specification (FIG. 17a). We tested whether blastoids co-opted this mechanism. Strikingly, atypical Protein Kinase C (aPKC) and F-actin expression domains appeared co-aligned in outer cells that also accumulated the Hippo downstream effector YAP1 in nuclei (FIG. 17b, c). YAP1 nuclear location correlated with GATA2/3 expression, contrasted with NANOG expression, and became restricted to TE analogs (FIG. 10b & FIG. 17d, e). A aPKC inhibitor (CRT0103390) largely prevented YAP1 nuclear accumulation, decreased the number of GATA3+ cells and prevented blastoid formation (FIG. 17f-h). Per contra, ligands of LPA receptors (LPA and NAEPA) that inhibit the Hippo pathway enhanced blastoid formation (FIG. 10c & FIG. 17i). Because Hippo pathway inhibition frees YAP1 to enter the nucleus, we tested whether genetically engineered levels and functions of YAP1 impacted morphogenesis. Overexpression of wildtype or constitutively active forms of YAP1 (5SA) accelerated cavitation (FIG. 10d). The interaction between YAP1 and TEAD transcription factors is necessary for down-stream gene regulation. Accordingly, over-expression of YAP1 comprising a mutation in the TEAD binding site (S94A) did not affect cavitation (FIG. 10d & FIG. 17j), and Verteporfin, a drug that disrupts the YAP1-TEAD interaction, prevented blastoid formation (FIG. 17k). Cavity morphogenesis occurred through the apparent coalescence of multiple fluid-filled cavities (FIG. 17l). Aquaporin 3 (AQP3), the water transporter most highly expressed in human blastocysts, was initially visible in all cells (36 hours) and then restricted to TE analogs (96 hours) (FIG. 17m). Thus, similar to human blastocysts, blastoids trophoblast specification and morphogenesis depends on aPKC, inhibition of the Hippo pathway, nuclear translocation of YAP1, and its ability to bind TEAD transcription factors.

### Example 11: Adequate Developmental Sequence

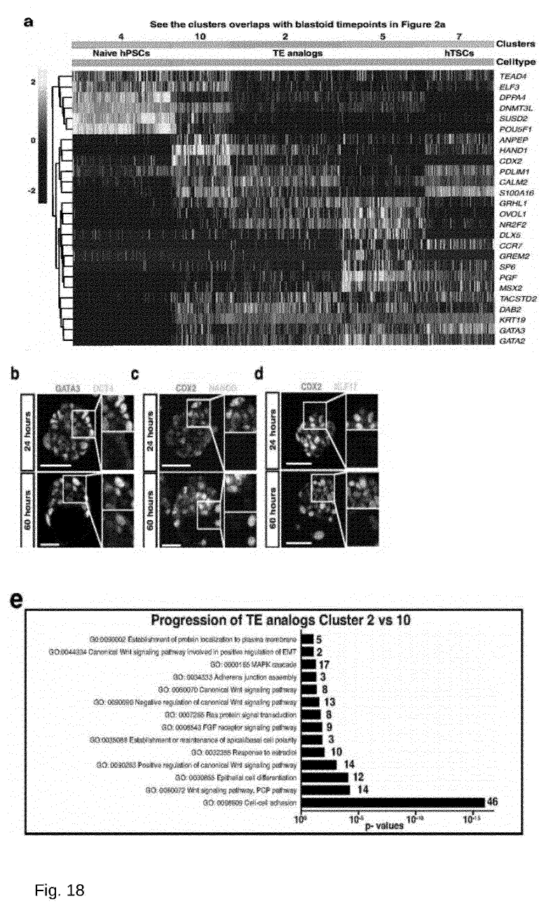

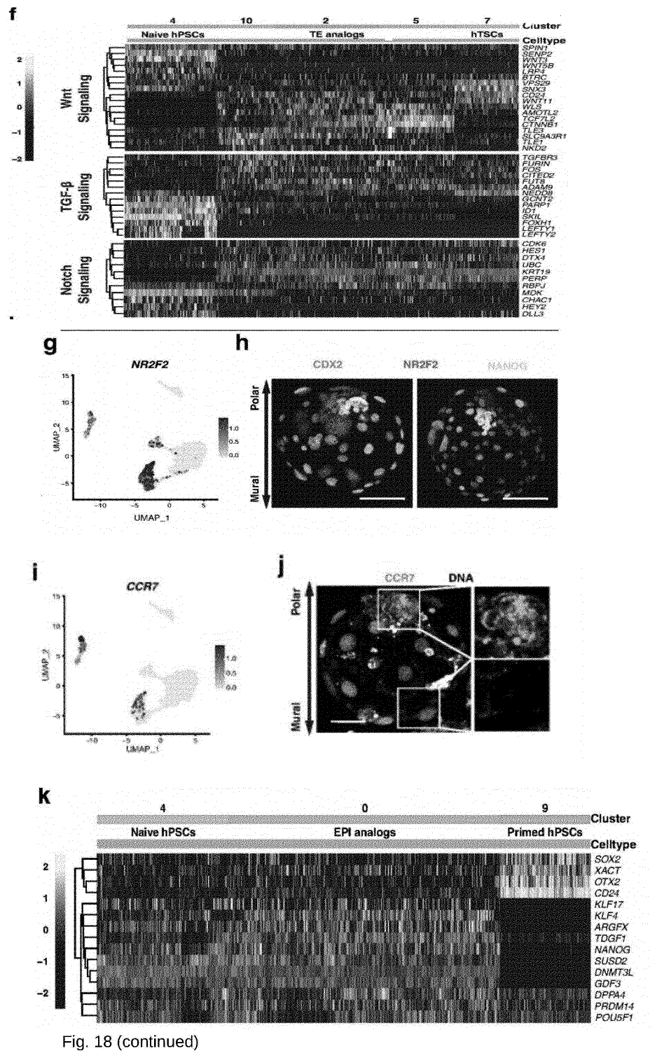

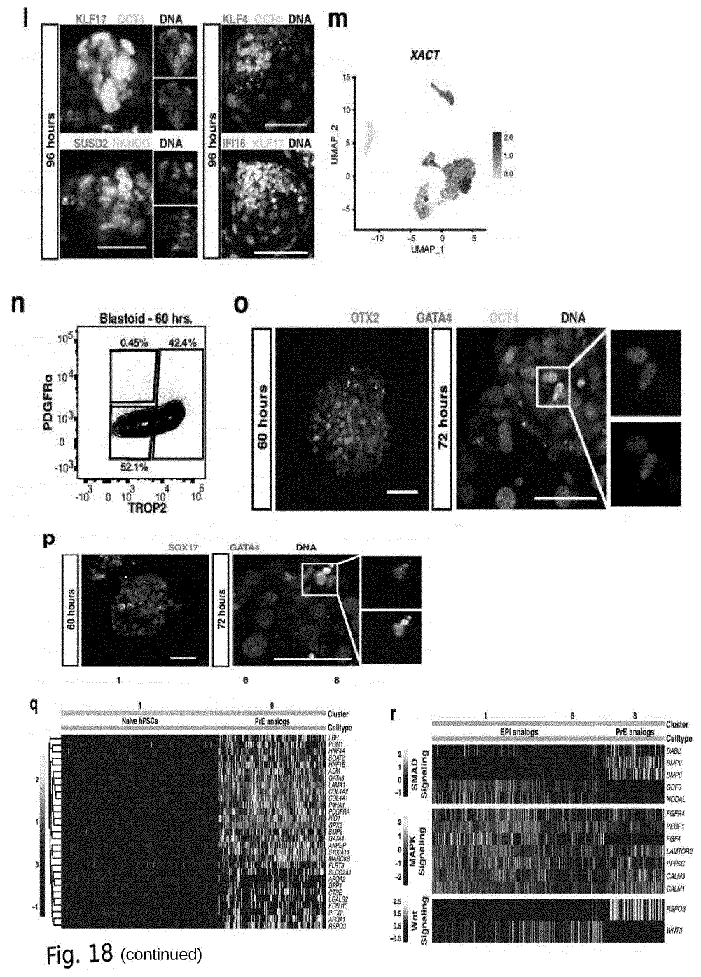

In blastocysts, trophoblasts appear first (days 5-6, GATA2⁺/DAB2⁺) and PrE cells last (days 6-7, GATA6⁺/ADM⁺). This sequence is recapitulated in blastoids with trophoblasts forming first (<24 hours, DAB2⁺, CDX2⁺, and GATA2⁺/3⁺, FIG. 10e & FIG. 18a) while changing transcript levels related to PKC and Hippo signaling (AKAP12, CAPZB, ULK4, MOB1a, AMOT, AMOTL2, LATS2, TEAD1). At protein level, early TE-like cells first appeared YAP1^{nuclear}/GATA2⁺ (24 hours), then CDX2⁺/GATA3⁺, while maintaining KLF17/OCT4 expression but not NANOG (60 hours) (FIG. 18b-d). Subsequently, OCT4 became undetectable (FIG. 8g & FIG. 12i). Genes associated with SMAD, ERK, Notch, and Wnt signaling pathways were regulated during this process (FIG. 18e, f). Finally, polar trophoblast analogs matured as marked by expression of OVOL1, GREM2, CCR7, SP6, and NR2F2 (FIG. 10f & FIG. 18g-j), up-regulation of NR2F2 and CCR7, and down-regulation of CDX2 (FIG. 10f & FIG. 18h, j). The transcriptome of EPI analogs maintained core blastocyst markers (POU5F1, NANOG, KLF17, SUSD2, KLF4, ARGFX, GDF3) (FIG. 10e & FIG. 18k, l), while undergoing an overtime progression characterized by regulation of Nodal (NODAL, LEFTY1/2) and mTOR signaling-related genes (LAMTOR1/4/5, XBP1, SEC13, MLST8), and of X chromosome activation-related gene XACT (FIG. 18k-m). Then, PrE analogs appeared (<60 hours) and GATA4, OTX2, and SOX17 were detected (72 hours) (FIG. 10e & FIG. 18n-p). Early PrE marker genes (GATA6, LBH, ADM, and LAMA1) were uniformly expressed among the PrE analogs, while some late PrE marker genes (CTSE, APOA1, PITX2 and SLCO2A1) were only expressed in a subpopulation, suggesting a progression toward late blastocyst state (FIG. 18q). By 96 hours, mature PrE analogs had regulated SMAD (NODAL, BMP2/6, GDF3, ID1/2) and Wnt signaling-related transcripts (WNT3, RSPO3, LBH) and were enriched in transcripts controlling extracellular matrix organization (LAMA1, LAMB1, LAMC1, COL4A1/2), endodermal and epithelial differentiation (FIG. 18q, r). Altogether, blastoid lineages specified and progressed in accordance with the sequence of blastocyst development.

### Example 12: Distinct Interactions with Endometrial Cells

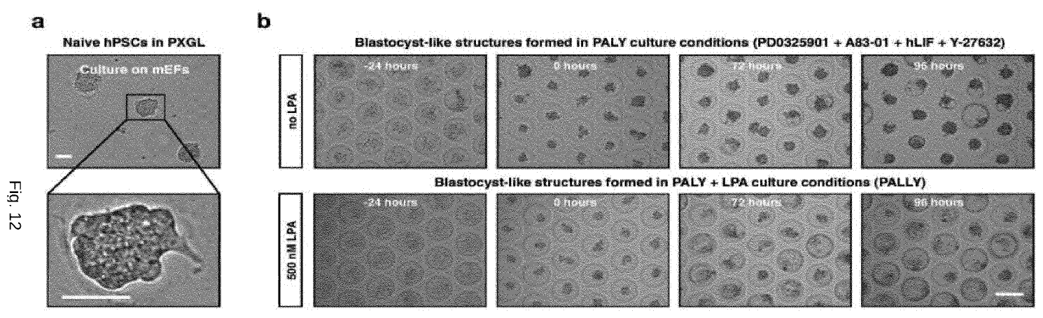

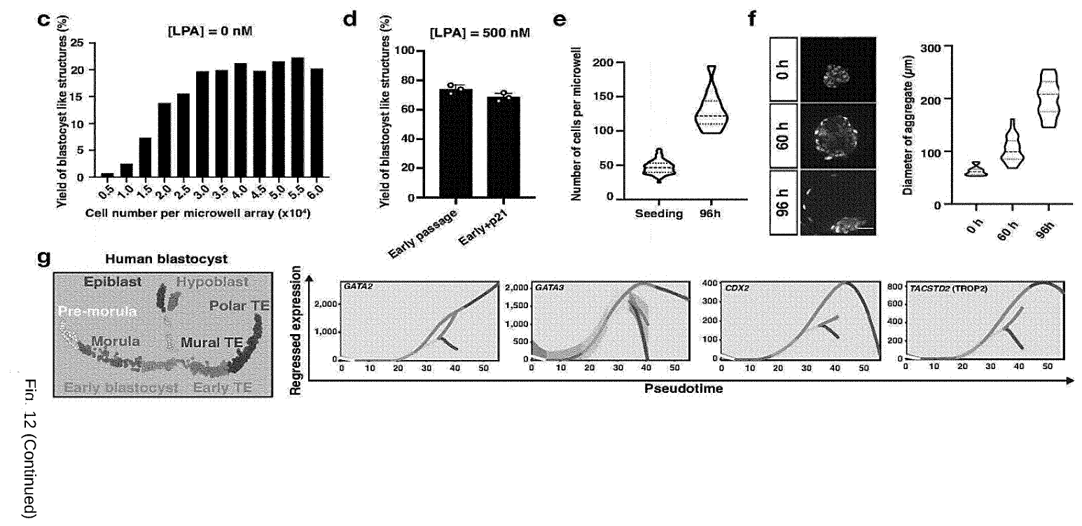

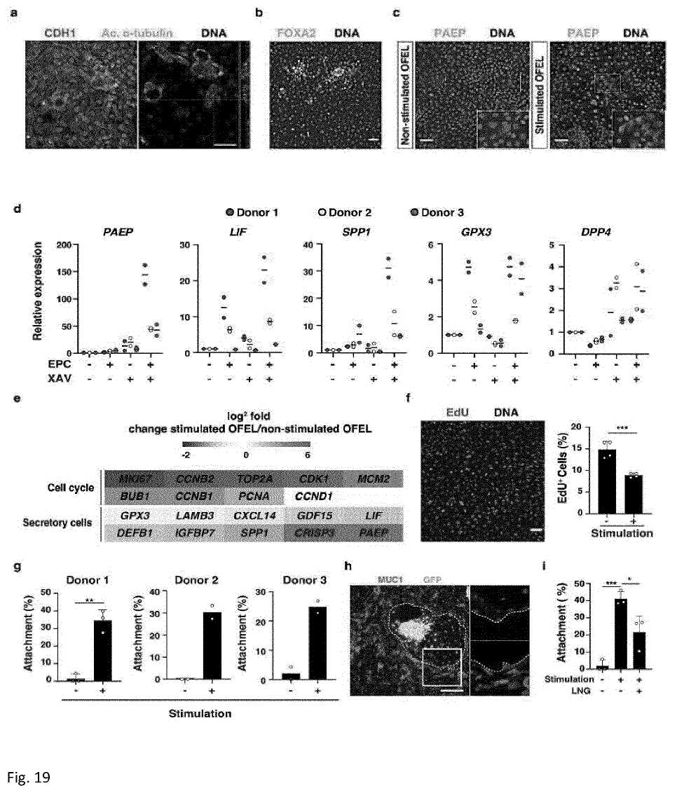

Human blastocysts initiate implantation in utero (days 7-9) via apposition and attachment of a polar TE to a receptive endometrium (FIG. 11a, left). We wondered whether blastoids could model this interaction. We seeded endometrial organoids (Boretto, M. et al. Development 144, 1775-1786 (2017)) in 2D to form an open-faced endometrial layer (OFEL) facilitating blastoid deposition (FIG. 11a, right). Subpopulations were positive for acetylated alpha tubulin marking ciliated epithelial cells (FIG. 19a) and FOXA2 marking glandular epithelial cells (FIG. 19b). The window of implantation opens upon estrogen (E2) and progesterone (P4) exposure and Wnt inhibition. Accordingly, OFELs responded to E2, P4, CAMP and XAV939 by upregulating the expression of genes marking the mid-secretory phase endometrium (FIG. 19c-e) and decreasing proliferation (FIG. 19e, f). Remarkably, blastoids deposited onto non-stimulated OFELs did not attach, while blastoids interacted with stimulated OFELs by attaching to and then repulsing endometrial cells as occurring in utero (FIG. 11b & FIG. 19g, h). The contraceptive Levonorgestrel impaired blastoid attachment (FIG. 191). We concluded that human blastoids are capable of interacting with an endometrium made receptive by hormones.

### Example 13: Epiblast Signals Gate-Keep Interactions

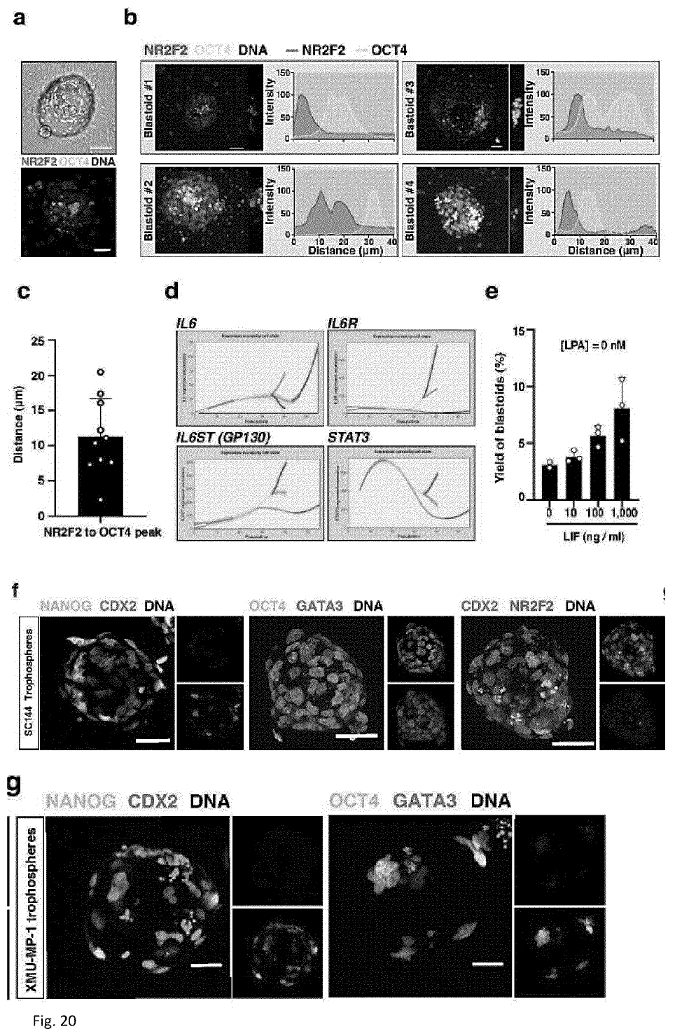

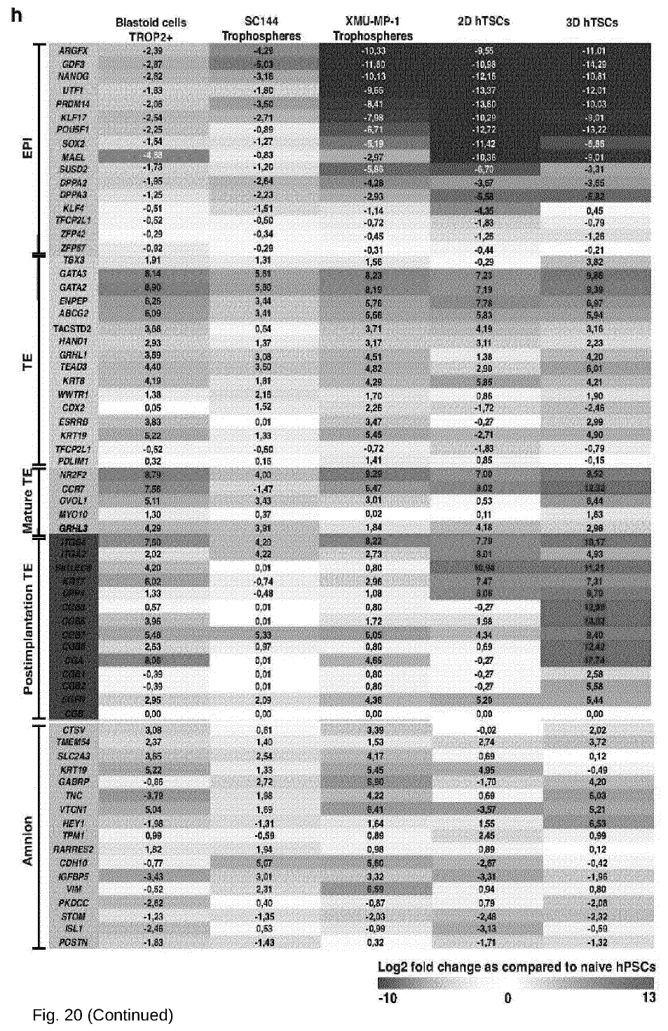

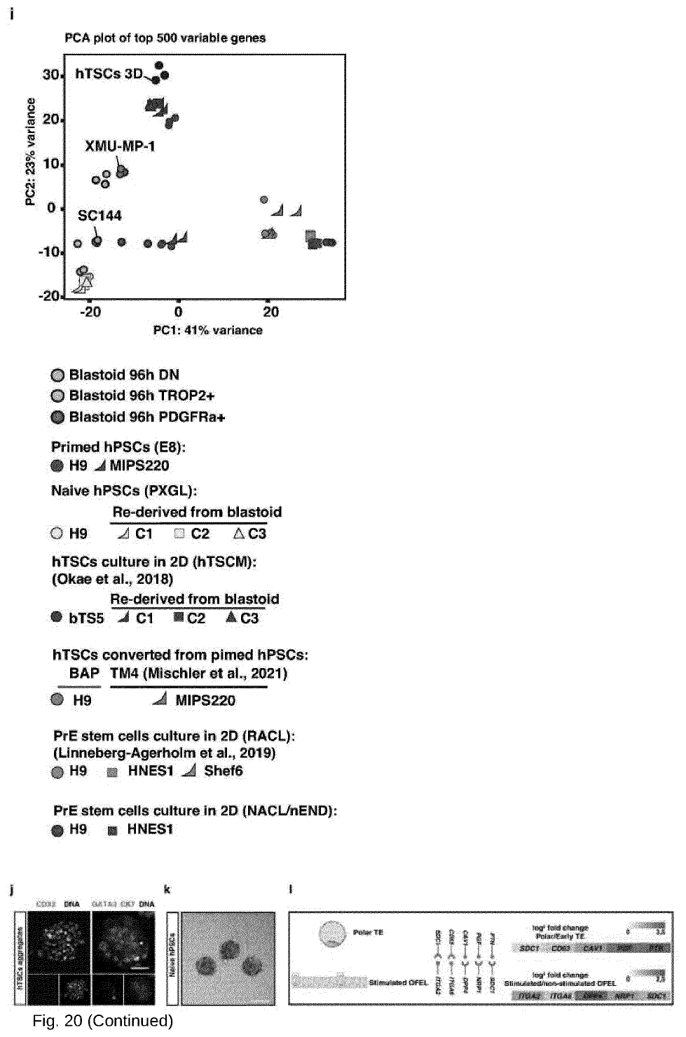

Human blastocysts attach to the endometrium via the polar trophectoderm defined by its contact with the EPI. Similarly, blastoids initiated attachment via this region (FIG. 11c, d & FIG. 20a-c). We then tested the importance of the polar/EPI interface by forming trophospheres (devoid of EPI). IL6 is highly expressed in the polar TE and transcripts for its receptor (IL6R, GP130) and effector (STAT3) abound in the EPI (FIG. 20d). Consistent with a role for STAT signaling in the EPI, blastoid formation efficiency increased with LIF concentration (FIG. 9e) while the addition of a GP130 inhibitor (SC144) yielded trophospheres (FIG. 11e & FIG. 20f). The presence of a potent inhibitor of the Hippo kinases MST1/2 (XMU-MP-1) also yielded trophospheres (FIG. 11e & FIG. 20g). The transcriptome of these trophospheres reflected early and late blastocyst trophoblasts respectively (FIG. 20h, i). Both trophosphere types failed to attach to OFELs (FIG. 11e), so did aggregates of hTSCs that reflect post-implantation cytotrophoblasts (CDX2⁻/CK7⁺) or of naive hPSCs (FIG. 11e & FIG. 20j, k). We concluded that signals from the EPI ensure polar trophoblast maturation, which permits interactions with endometrial cells. Based on transcriptome analysis and in utero data, we propose several pairs of molecules whose transcripts become more abundant upon endometrial cell stimulation and polar trophoblast maturation (FIG. 20l). These might mediate the first touch between blastocyst and uterus. Overall, we concluded that a pre-implantation polar-like state, whose maturation depends on EPI inductions, gate-keeps the blastocyst interactions with the endometrium.

### Example 14: Modeling Post-Stages (Day 13)

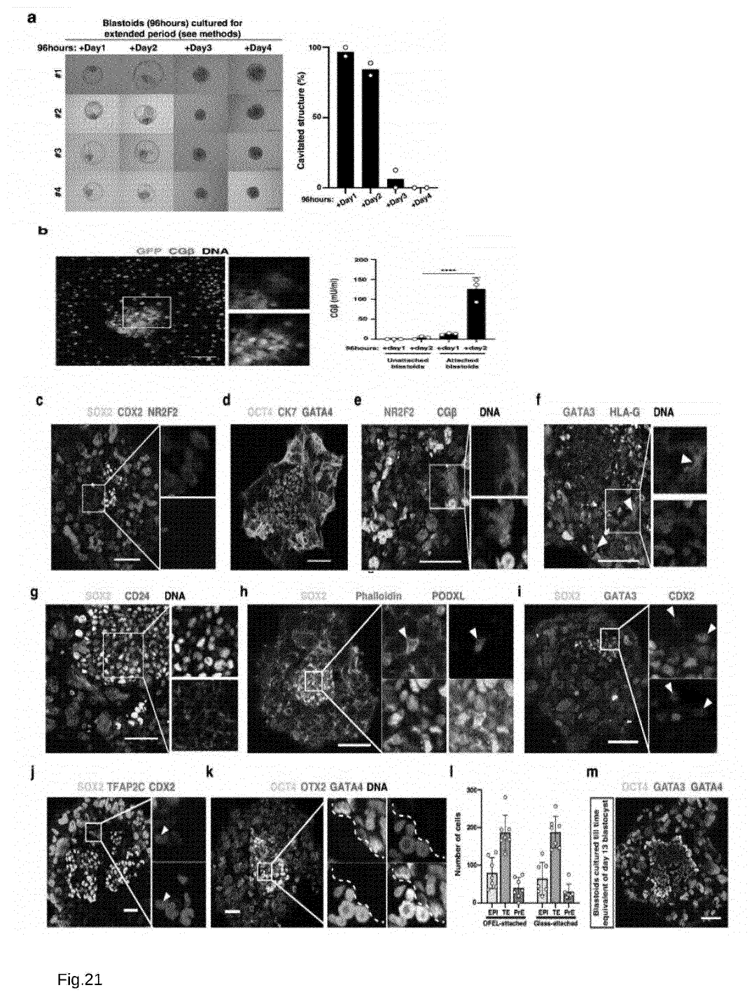

Blastoids morphology was stable for two days in peri-implantation culture conditions (FIG. 21a). Clinical pregnancy is characterized by the detection of Chorionic Gonadotropin B (CGB) hormone. Upon attachment, blastoids formed trophoblasts expressing CGB at levels detectable using standard pregnancy tests and ELISA (FIG. 11f & FIG. 21b). NR2F2⁺ polar trophoblast analogs proliferated and decreased CDX2 expression while upregulating the peri-implantation gene Cytokeratin 7 (CK7) (FIG. 21c, d). Some trophoblasts further differentiated into SCT and EVT expressing CGB and HLA-G respectively (FIG. 21e, f). EPI analogs maintained OCT4, SOX2, upregulated primed pluripotency marker CD24 (FIG. 11g & FIG. 21g), patterned cortical F-actin as during EPI epithelization, and some blastoids formed pro-amniotic-like cavities enriched with F-actin/PODXL⁺/aPKC⁺ (FIG. 11g & FIG. 21h). Also, a subpopulation in the periphery of the EPI analog expressed CDX2 along with SOX2 or TFAP2C, suggestive of early amnion analogs (FIG. 21i, j). PrE analogs were characterized by a restricted expression of OTX2 (FIG. 180, 21k). Upon prolonged culture (up to 6 days), the three lineages consistently expanded (FIG. 11h & FIG. 21l, m) until a time-equivalent of day 13, although, similar to blastocysts, their organization did not reflect that developmental stage.

### DISCUSSION

The fidelity, efficiency, scalability, and versatility of this model makes it relevant to study human blastocyst development and implantation. The inventive blastoids aid to identify therapeutic targets and contribute to preclinical modeling (e.g. IVF medium complements such as candidates LPA/NAEPA or contraceptives such as candidate SC144). Considering the proportionality (balancing the benefits and harms) and subsidiarity (pursuing goals using the morally least problematic means) of human embryology, blastoids represent an ethical opportunity complementing research using embryos.

Surprisingly, the stimulation of the aggregates of hPSCs with the 3 inhibitors not only leads to the formation of trophoblast cells, but to the concomitant formation of trophoblasts, epiblast, and primitive endoderm cells that spatially organize. The spontaneous organization of these 3 cell types is characterized by the formation of a trophoblast cyst that forms the outer layer and an inner fluid-filled cavity, and by the formation of a unique inner cluster of epiblast and primitive endoderm cells that remains attached to one side of the trophoblast cyst. As a direct consequence, the asymmetry created within the cyst by the presence of this unique inner cluster induces the local maturation of polar trophoblasts and defines the direction of the attachment to endometrial cells.

1. A method of generating a blastoid or a blastocyst-like cell aggregate comprising culturing an aggregate of human pluripotent stem cells (hPSCs) and trophoblast cells in a medium comprising a HIPPO pathway inhibitor in a 3D culture, wherein the aggregate of hPSCs and trophoblasts is generated by culturing aggregated hPSCs in a medium comprising a MEK inhibitor and a TGF-beta inhibitor.

2. The method of claim 1, wherein the aggregate of hPSCs and trophoblasts is generated by culturing aggregated hPSCs in a medium further comprising a HIPPO pathway inhibitor.

3. The method of claim 1, wherein the aggregated hPSCs are formed by seeding hPSCs and aggregating the seeded hPSCs by culturing in a growth medium for 0 to 64 hours, and/or wherein the growth medium comprises a ROCK inhibitor.

4. The method of claim 3, wherein the seeded hPSCs have been treated with one of a MEK inhibitor and a PKC inhibitor, further comprising a Wnt inhibitor and/or a STAT agonist, wherein treatment is in a 2D culture.

5. The method of claim 4, wherein the PKC inhibitor is selected from Gö6983 and Ro-31-8425.

6. The method of claim 1, wherein the HIPPO pathway inhibitor is a ligand of the lysophosphatidic acid (LPA) receptor; and/or the MEK inhibitor is PD0325901; and/or the TGF-beta inhibitor is A83-1 or SB431542.

7. The method of claim 1, comprising culturing the cells at least until formation of a trophectoderm-like tissue, an epiblast-like tissue and a hypoblast-like tissue out of the aggregate of hPSCs and trophoblasts; and/or culturing the cells at least until formation of a three-dimensional cell aggregate with an overall diameter of at least 100 μm, formed by an outer epithelial monolayer of trophoblast-like cells surrounding a fluid-filled cavity and at least one inner cluster of cells comprising epiblast-like and hypoblast-like cells.

8. The method of claim 1, further comprising the stimulation of endometrial cells with a compound selected from estrogen, estrone, estriol, ethinyl estradiol, 17α-ethylnylestradiol, mestranol, progesterone, a progestin, CAMP, and a Wnt-inhibitor, and the seeding of the seeding of the blastoid onto the layer of stimulated endometrial cells that allows the blastoid to attach and invade the layer of endometrial cells.

9. The method of claim 1 for testing or screening a candidate compound and/or candidate genetic alteration on having an effect at blastoid formation and/or implantation of a blastoid into a layer of endometrial cell comprising treating the aggregate with at least one candidate compound and/or providing the aggregate with at least one candidate genetic alteration.

10. A kit suitable for culturing a blastoid, comprising a HIPPO pathway inhibitor, a MEK inhibitor, and a TGF-beta inhibitor.

11. A blastoid obtainable by a method of claim 1.

12. The blastoid of claim 11, comprising an outer epithelial monolayer of trophoblast-like cells surrounding at least one fluid-filled cavity and at least one inner cluster of cells comprising epiblast-like and hypoblast-like cells, wherein the outer epithelial monolayer comprises polar trophoblasts that express NR2F2.

13. A blastoid comprising an outer epithelial monolayer of trophoblast-like cells surrounding at least one fluid-filled cavity and at least one inner cluster of cells comprising epiblast-like and hypoblast-like cells, wherein the outer epithelial monolayer comprises polar trophoblasts that express NR2F2.

14. An in vitro method of increasing the potential of implanting a blastoid or blastocyst into a layer of endometrial cells, comprising treating the blastoid or blastocyst with a Wnt inhibitor, and contacting the blastoid or blastocyst with the layer of endometrial cells.

15. A Wnt inhibitor for use in a method of increasing the chance of a blastocyst implantation, comprising contacting the blastocyst with an endometrium in the presence of the Wnt inhibitor, or comprising stimulating the endometrium with a Wnt inhibitor before transferring the blastocyst to the endometrium.

16. A method of producing a blastocyst, comprising treating an embryo in an early stage, selected from the 1-cell stage, 2-cells stage, 4-cells stage, 8-cells stage or 16-cells stage or morula stage or blastocyst stage until a mature blastocyst stage, with a HIPPO pathway inhibitor, and letting the embryo in a 1-cell stage, 2-cells stage, 4-cells stage, 8-cells stage or 16-cells stage or morula stage grow into the blastocyst stage or letting the embryo in the blastocyst stage grow into a more mature blastocyst stage.

17. A method of contraception, comprising administering to a patient and/or contacting an embryo in vivo with a MST1/2 inhibitor.
